(19) United States
(12) Patent Application Publication
(10) Pub. No.: US 2025/0361399 A1
(43) Pub. Date: Nov. 27, 2025

(54) FLUORESCENT DYES WITH LARGE STOKES SHIFT

(71) Applicant: Roche Molecular Systems, Inc., Pleasanton, CA (US)

(72) Inventor: Alexander Nierth, Dublin, CA (US)

(21) Appl. No.: 18/875,039

(22) PCT Filed: Jun. 26, 2023
(86) PCT No.: PCT/EP2023/067223

§ 371 Date: Dec. 13, 2024

Related U.S. Application Data
(60) Provisional application No. 63/356,433, filed on Jun. 28, 2022.

(51) Int. Cl. C09B 11/24; G01N 33/58

(52) U.S. Cl. CPC C09B 11/24; G01N 33/582

(57) ABSTRACT

The present disclosure relates, in general, to novel and easily accessible fluorescent compounds with large Stokes shift (LSS) and thermostable fluorescence for expanding the multiplexing capabilities of fluorescence-based nucleic acid detection technologies. Moreover, conjugates, probes and FRET pairs comprising these fluorescent compounds as well as methods for amplification and detection of a target nucleic acid utilizing these fluorescent compounds and methods of labeling are also provided.

### CROSS REFERENCE TO RELATED APPLICATIONS

The present disclosure claims the benefit of the filing date of U.S. Provisional Patent Application No. 63/356,433 field on Jun. 28, 2022, the disclosure of which is hereby incorporated by reference herein in its entirety.

### FIELD OF THE DISCLOSURE

The present disclosure relates to novel and easily accessible fluorescent compounds with large Stokes shift (LSS) and thermostable fluorescence for expanding the multiplexing capabilities of fluorescence-based nucleic acid detection technologies.

### BACKGROUND OF THE DISCLOSURE

The polymerase chain reaction (PCR) has become a ubiquitous tool of biomedical research, disease monitoring and diagnostics. Amplification of nucleic acid sequences by PCR is described in U.S. Pat. Nos. 4,683,195, 4,683,202, and 4,965,188. PCR is now well known in the art and has been described extensively in the scientific literature. See PCR Applications, ((1999) Innis et al., eds., Academic Press, San Diego), PCR Strategies, ((1995) Innis et al., eds., Academic Press, San Diego); PCR Protocols, ((1990) Innis et al., eds., Academic Press, San Diego), and PCR Technology, ((1989) Erlich, ed., Stockton Press, New York). A “real-time” PCR assay is able to simultaneously amplify and detect and/or quantify the starting amount of the target sequence. The basic TaqMan® real-time PCR assay using the 5′-to-3′ nuclease activity of the DNA polymerase is described in Holland et al., (1991) Proc. Natl. Acad. Sci. 88:7276-7280 and U.S. Pat. No. 5,210,015. A real-time PCR without the nuclease activity (a nuclease-free assay) has been described in U.S. Patent Publication No. 20100143901A1. The use of fluorescent probes in real-time PCR is described in U.S. Pat. No. 5,538,848.

A typical real-time PCR protocol with fluorescent probes involves the use of a labeled probe, specific for each target sequence. The probe is preferably labeled with one or more fluorescent moieties, which absorb and emit light at specific wavelengths. Upon hybridizing to the target sequence or its amplicon, the probe exhibits a detectable change in fluorescent emission as a result of probe hybridization or hydrolysis.

The major challenge of the real-time assay however remains the ability to analyze numerous targets in a single tube. In virtually every field of medicine and diagnostics, the number of loci of interest increases rapidly. For example, multiple loci must be analyzed in forensic DNA profiling, pathogenic microorganism detection, multi-locus genetic disease screening and multi-gene expression studies, to name a few.

Commercial, fluorescence-based devices for automated polymerase chain reaction (PCR) can detect multiple targets in a single reaction vessel (multiplexing) by distinguishing light from differently colored fluorophores. The dyes are selected in a way to minimize their spectral overlap.

Every fluorophore in the ensemble can be excited with light at or near the absorption maximum and the emitted light (fluorescence) is detected at or near the fluorescence maximum. By limiting the range of wavelengths (band) for excitation and emission with optical filters, individual fluorophores can be distinguished. The specific combination of an excitation band and a simultaneously detected emission band defines an optical channel, each allowing for the identification of one PCR target.

The achievable maximum number of optical channels depends on numerous interrelated factors, such as available spectral range, excitation light intensity, fluorophore brightness, fluorophore spectral width, filter bandwidth, and detector sensitivity. State-of-the-art PCR devices with fluorescence-based detection technologies use between four and up to six optical filters per excitation and emission pathway. Therefore, with standard fluorophores, four to six individual PCR targets can be distinguished.

### BRIEF SUMMARY OF THE DISCLOSURE

The present disclosure is directed to dyes having a large Stokes shift, such as a Stokes shift of about 50 nm or more, of about 60 nm or more, of about 70 nm or more, of about 80 nm or more, of about 90 nm or more, etc.). Herein, it has been determined that the incorporation of specific linker moieties into the dyes of the present disclosure allow for facile tuning of their spectroscopic properties, such as their excitation and emission wavelengths. Moreover, by choosing suitable linker moieties the dyes of the present disclosure may be converted to their respective activated derivatives, such as their respective NHS-esters; or modified to incorporate a functional group capable of participating in a click-chemistry chemistry reaction for bio-molecular labeling. Additionally, the introduction of a protecting group allows the derivatization of the dyes into phosphoramidites, such as those that are compatible with solid-phase synthesis of nucleic acids and phosphoramidite chemistry.

Further, it has been determined that the dyes of the present disclosure exhibit excellent brightness in combination with thermostable fluorescence. It has also been surprisingly discovered that the dyes of the present disclosure are easily accessible from inexpensive starting materials in a single, high-yielding reaction step. These and other benefits are described further herein.

A first aspect of the present disclosure is a compound having Formula (I):

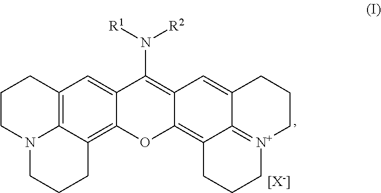

wherein
R¹ is H or a protecting group;
R² is a C₁-C₈ branched or unbranched alkyl, branched or unbranched heteroalkyl, or cycloalkyl group which is substituted with one or more of -Me, -Et, —CO₂⁻, —CO₂-(thiol reactive group), —CO₂-(amine reactive group), —CO₂-(carboxy reactive group), —C₂—CO₂⁻, —C₂—CO₂-(thiol reactive group), —C₂—CO₂-(amine reactive group), —C₂—CO₂-(carboxy reactive group), —OH, -phosphoramidite, —O-phosphoramidite, -D, a halogen, or a group capable of participating in a “click chemistry” reaction; and
[X]⁻ is a counter anion, provided when R² has a negative charge, [X]⁻ is not present.

In some embodiments, R¹ is 9-fluorenylmethyl carbamate, t-Butyl carbamate, benzyl carbamate, acetamide, trifluoroacetamide, benzylamine, triphenylmethylamine, monomethoxytrityl (MMT), DMS, and p-toluenesulfonamide. In some embodiments, R¹ is H. In some embodiments, a first carbon atom of R² is a primary carbon atom. In some embodiments, a first carbon atom of R² is a secondary carbon atom. In some embodiments, a first carbon atom of R² is a tertiary carbon atom. In some embodiments, the group capable of participating in the “click chemistry reaction” is selected from the group consisting of a bicyclo[6.1.0]nonyne) group (“BCN”), dibenzocyclooctyne (“DBCO”), alkene, trans-cycloctene (“TCO”), maleimide, an aldehyde, a ketone, an azide, a tetrazine, a thiol, a 1,3-nitrone, a hydrazine, and a hydroxylamine. In some embodiments, the group capable of participating in the “click chemistry reaction” is DBCO, TCO, or azide. In some embodiments, the thiol reactive group is selected from the group consisting of a haloacetyl, a maleimide, an iodoacetamide, an aziridine, an acryloyl, an arylating agent, a vinylsulfone, a methanethiosulfonate, a pyridyl disulfide, and a TNB-thiol. In some embodiments, the thiol reactive group is a maleimide. In some embodiments, the amine reactive group is selected from the group consisting of an NHS ester, an isothiocyanate, an acyl azide, a sulfonyl chloride, a sulfodichlorophenol, pentafluorophenol, tetrafluorophenol, 4-sulfo-2,3,5,6-tetrafluorophenyl, an aldehyde, a glyoxal, an epoxide, an oxirane, a carbonate, an aryl halide, a fluorophenol ester, a sulfochlorophenol, a carbodiimide, a phthalimide, a benzotriazole, an imidoester, and an anhydride. In some embodiments, the carbonyl-reactive group is selected from the group consisting of a hydrazine, a hydrazine derivative, and an amine. In some embodiments, R² is selected from the group consisting of a C₁-C₈ branched or unbranched alkyl, branched or unbranched heteroalkyl, or cycloalkyl group which is substituted with a —CO₂-maleimide or a —C₂—CO₂-maleimide; a C₁-C₈ branched or unbranched alkyl, branched or unbranched heteroalkyl, or cycloalkyl group which is substituted with a —CO₂—NHS ester or a —C₂—CO₂—NHS ester; and a C₁-C₈ branched or unbranched alkyl, branched or unbranched heteroalkyl, or cycloalkyl group which is substituted with a —CO₂-hydrazine or a —C₂—CO₂— hydrazine. In some embodiments, R² is a C₁-C₆ branched or unbranched alkyl, branched or unbranched heteroalkyl, or cycloalkyl group which is substituted with one or more of -Me, -Et, —CO₂⁻, —CO₂-(thiol reactive group), —CO₂-(amine reactive group), —CO₂-(carboxy reactive group), —C₂—CO₂⁻, —C₂—CO₂-(thiol reactive group), —C₂—CO₂-(amine reactive group), —C₂—CO₂-(carboxy reactive group), —OH, -phosphoramidite, —O-phosphoramidite, -D, a halogen, or a group capable of participating in a “click chemistry” reaction. In some embodiments, R² is a C₁-C₆ branched or unbranched alkyl group substituted with BCN, DBCO, azide, or TCO. In some embodiments, R² is a C₁-C₆ branched or unbranched alkyl group substituted with one or more of -Me, -Et, —CO₂⁻, —OH, -D, or a halogen. In some embodiments, R² is selected from:

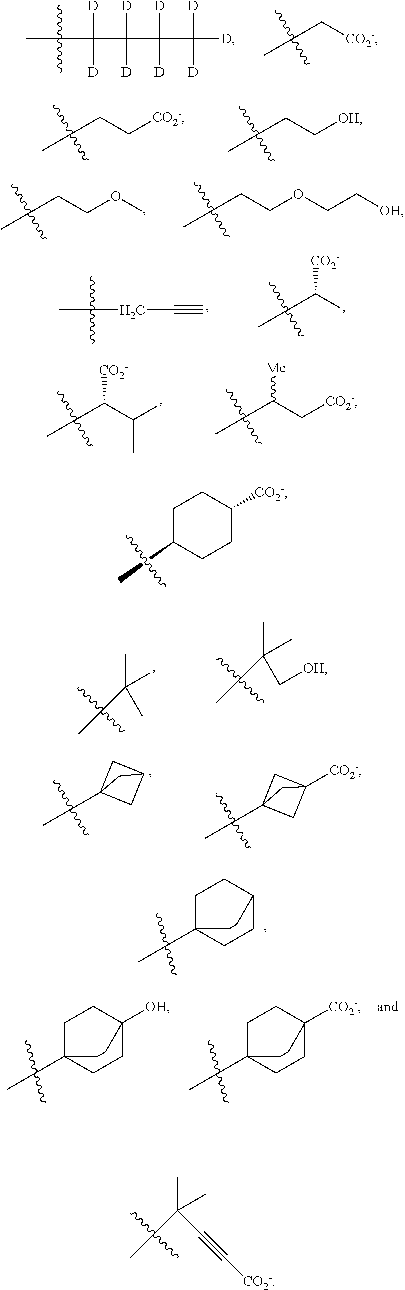

In some embodiments, R² is -phosphoramidite or —O-phosphoramidite. In some embodiments, R¹ is 9-fluorenylmethyl carbamate, t-Butyl carbamate, benzyl carbamate, acetamide, trifluoroacetamide, benzylamine, triphenylmethylamine, monomethoxytrityl (MMT), DMS, and p-toluenesulfonamide; and R² is -phosphoramidite or —O-phosphoramidite.

A second aspect of the present disclosure is compound selected from the group consisting of:

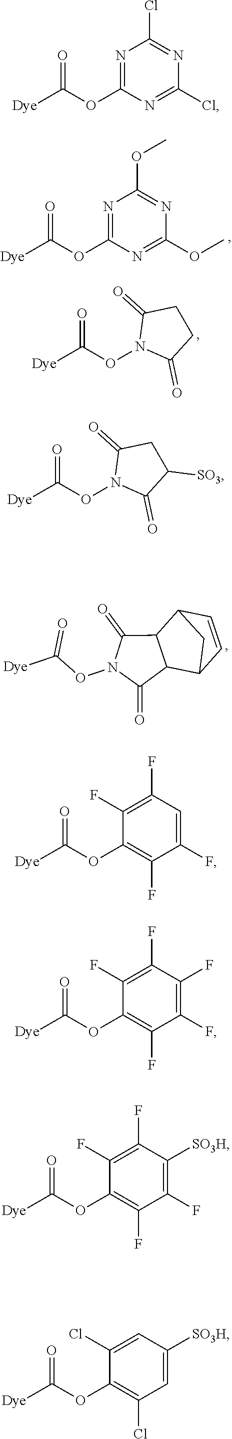

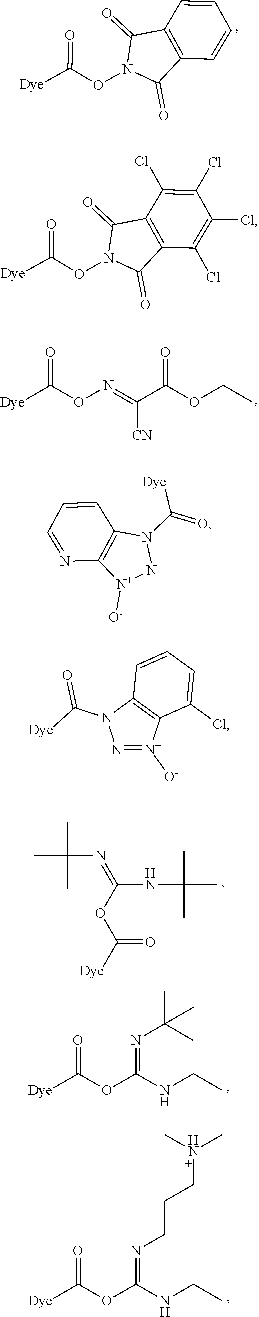

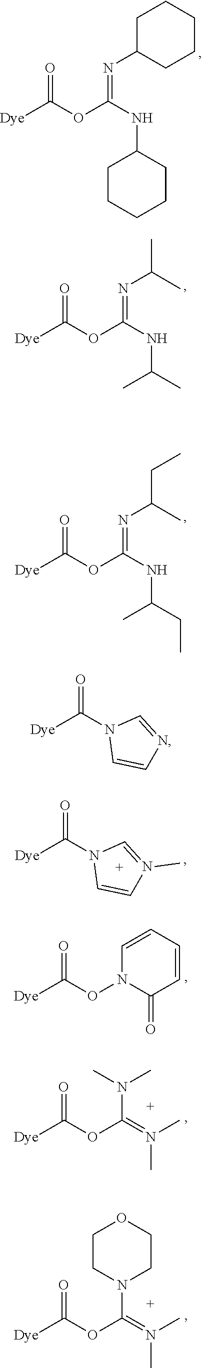

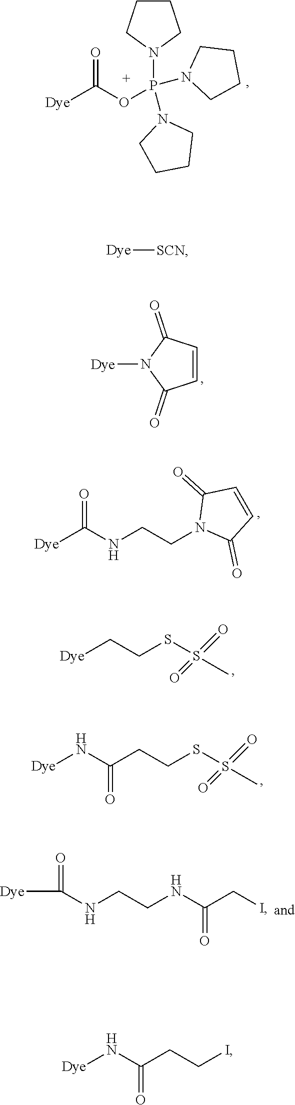

where “Dye” is

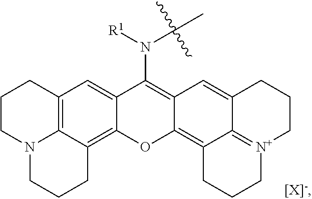

and wherein [X]⁻ is a counter anion.

A third aspect of the present disclosure is a compound selected from the group consisting of:

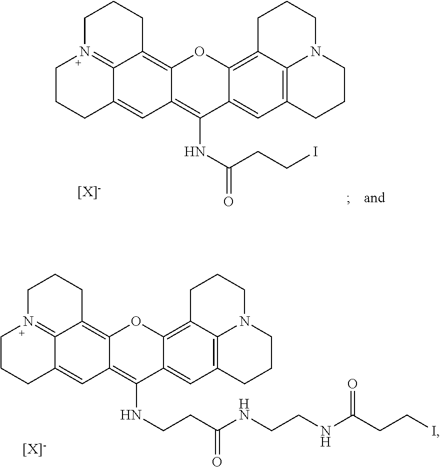

wherein [X]⁻ is a counter anion.

A fourth aspect of the present disclosure is a compound having Formula (IA):

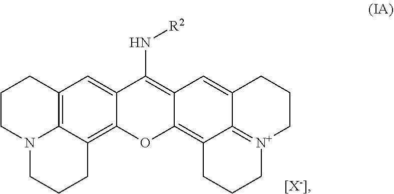

R² is a C₁-C₈ branched or unbranched alkyl, branched or unbranched heteroalkyl, or cycloalkyl group which is substituted with one or more of -Me, -Et, —CO₂⁻, —CO₂-(thiol reactive group), —CO₂-(amine reactive group), —CO₂-(carboxy reactive group), —C₂—CO₂⁻, —C₂—CO₂-(thiol reactive group), C₂—CO₂-(amine reactive group), —C₂—CO₂-(carboxy reactive group), —OH, phosphoramidite, —O-phosphoramidite, -D, a halogen, or a group capable of participating in a “click chemistry” reaction; and [X]⁻ is a counter anion, provided when R₂ has a negative charge, [X]⁻ is not present.

In some embodiments, R² is selected from the group consisting of a C₁-C₈ branched or unbranched alkyl, branched or unbranched heteroalkyl, or cycloalkyl group which is substituted with a —CO₂— maleimide or a —C₂—CO₂-maleimide; a C₁-C₈ branched or unbranched alkyl, branched or unbranched heteroalkyl, or cycloalkyl group which is substituted with a —CO₂—NHS ester or a —C₂—CO₂—NHS ester; and a C₁-C₈ branched or unbranched alkyl, branched or unbranched heteroalkyl, or cycloalkyl group which is substituted with a —CO₂-hydrazine or a —C₂—CO₂-hydrazine. In some embodiments, R² is a C₁-C₈ branched or unbranched alkyl group substituted with one or more of -Me, -Et, —CO₂⁻, —OH, -phosphoramidite, —O-phosphoramidite, -D, a halogen, or a group capable of participating in a “click chemistry” reaction. In some embodiments, the group capable of participating in a “click chemistry” reaction is selected from the group consisting of azide, DBCO, TCO, maleimide, and tetrazine. In some embodiments, R² is a C₁-C₈ branched or unbranched alkyl group substituted with one or more of -Me, -Et, —CO₂⁻, —OH, a halogen, -D, or a group capable of participating in a “click chemistry” reaction. In some embodiments, R² is a C₁-C₈ branched or unbranched alkyl group substituted with one or more of -Me, -Et, —CO₂⁻, —OH, a halogen, or -D. In some embodiments, R² is -phosphoramidite or —O-phosphoramidite. In some embodiments, R² is selected from:

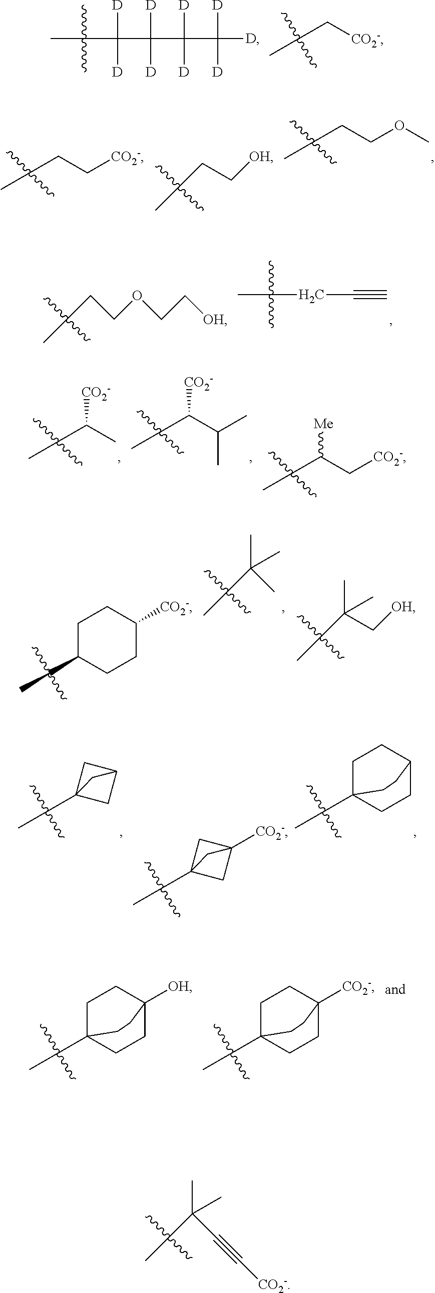

In some embodiments, the compounds of the fourth aspect of the present disclosure have a Stokes shift of at least about 70 nm. In some embodiments, the compounds of the fourth aspect of the present disclosure have a Stokes shift of at least about 80 nm. In some embodiments, the compounds of the fourth aspect of the present disclosure have a Stokes shift of at least about 90 nm. In some embodiments, the compounds of the fourth aspect of the present disclosure are thermally stable over a temperature ranging from about 25° C. to about 100° C. In some embodiments of the third and fourth aspect, [X]⁻ is selected from the group consisting of chloride, bromide, iodide, sulfate, benzene sulfonate, p-toluenesulfonate, p-bromobenzenesulfonate, methanesulfonate, trifluoromethanesulfonate, phosphate, perchlorate, tetrafluoroborate, hexafluorophosphate, tetraphenylboride, nitrate; and anions of aromatic or aliphatic carboxylic acids. A fifth aspect of the present disclosure is a conjugate comprising (i) a specific binding entity, and (ii) a dye moiety derived from a compound having any one of Formulas (I), (IA), and (IB) (such as any of those compounds described herein). In some embodiments, specific binding entity is a protein. In some embodiments, the protein is an antibody, an antibody fragment, or an enzyme. In some embodiments, the specific binding entity is an oligonucleotide. In some embodiments, the oligonucleotide comprises between about 5 and about 60 mer. In some embodiments, the dye moiety is coupled to a 5′ end of the oligonucleotide. In some embodiments, the dye moiety is coupled to a 3′ end of the oligonucleotide. In some embodiments, the dye moiety is derived from any one of the compounds having Formula (IA).

A sixth aspect of the present disclosure is a conjugate comprising (i) a hapten, and (ii) a dye moiety derived from a compound having any one of Formulas (I), (IA), and (IB). In some embodiments, the hapten is a pyrazole; a nitrophenyl compounds; a benzofurazan; a triterpene; a urea; a thiourea; a rotenone or a rotenone derivative; an oxazole; a thiazole; a coumarin or a coumarin derivative; or a cyclolignan.

A seventh aspect of the present disclosure is a conjugate having Formula (II).

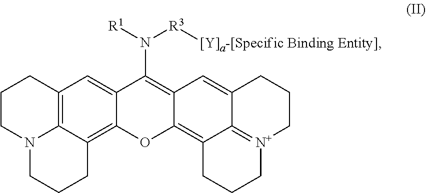

wherein
R¹ is H or a protecting group;
R³ is a C₁-C₈ alkyl, heteroalkyl, or cycloalkyl group which is substituted with one or more of -Me, -Et, —CO₂—, —C₂—CO₂—, -D, or a halogen;
the “Specific Binding Entity” is an oligonucleotide or a protein;
Y is a branched or unbranched, substituted or unsubstituted, saturated or unsaturated aliphatic or aromatic group having between 2 and about 40 carbon atoms, and optionally having one or more heteroatoms selected from O, N, or S; and a is 0, 1, or 2.

In some embodiments, the protein is an antibody, e.g., a primary antibody or a secondary antibody. In some embodiments, the oligonucleotide comprises between about 5 mer and about 60 mer. In some embodiments, the oligonucleotide comprises between about 5 mer and about 40 mer. In some embodiments, the oligonucleotide comprises between about 5 mer and about 20 mer. In some embodiments, Y is a branched or unbranched, linear, or cyclic, substituted or unsubstituted, saturated or unsaturated, group having between 2 and about 30 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 25 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 20 carbon atoms, and optionally having one or more heteroatoms selected from O, N, or S. In some embodiments, Y has the structure of Formula (IIIC):

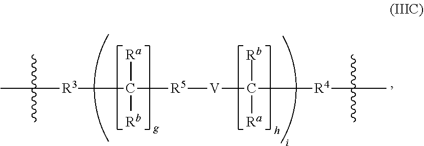

wherein each of R³ and R⁴ are independently a bond or a group selected from carbonyl, amide, imide, ester, ether, —NH, —N—, thione, or thiol;
R⁵ is a bond, a C₁-C₁₂ alkyl or heteroalkyl group including, and wherein R⁵ may include a carbonyl, an imine, or a thione;
R^{a} and R^{b} are independently H or methyl;
g and h are independently 0 or an integer ranging from 1 to 4; i is 0, 1 or 2.

In some embodiments, R³ is a C₁-C₆ alkyl, heteroalkyl, or cycloalkyl group which is substituted with one or more of -Me, -Et, —CO₂—, —C₂—CO₂—, -D, or a halogen. In some embodiments, R³ is a C₁-C₄ alkyl, heteroalkyl, or cycloalkyl group which is substituted with one or more of -Me, -Et, —CO₂—, —C₂—CO₂—, -D, or a halogen.

An eight aspect of the present disclosure is a conjugate having any one of Formulas (IIC) or (IID):

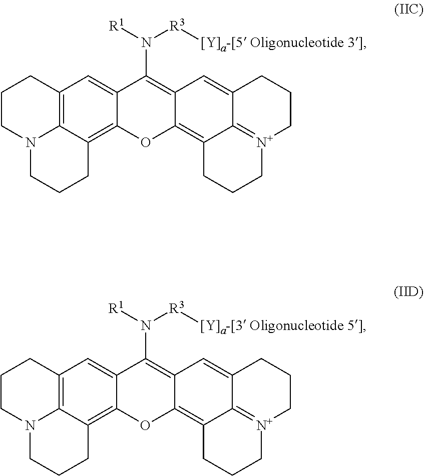

wherein
R¹ is H or a protecting group;
R³ is a C₁-C₈ alkyl, heteroalkyl, or cycloalkyl group which is substituted with one or more of -Me, -Et, —CO₂—, —C₂—CO₂—, -D, or a halogen;
Oligonucleotide is an oligonucleotide having between about 5 and about 60 mer;
Y is a branched or unbranched, substituted or unsubstituted, saturated or unsaturated aliphatic or aromatic group having between 2 and about 40 carbon atoms, and optionally having one or more heteroatoms selected from O, N, or S; and
a is 0, 1, or 2.

In some embodiments, a first carbon atom of R³ is a primary carbon atom. In some embodiments, a first carbon atom of R³ is a secondary carbon atom. In some embodiments, a first carbon atom of R³ is a tertiary carbon atom. In some embodiments, Y is a branched or unbranched, linear, or cyclic, substituted or unsubstituted, saturated or unsaturated, group having between 2 and about 30 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 20 carbon atoms, and optionally having one or more heteroatoms selected from O, N, or S. In some embodiments, R¹ is H; and R³ is a C₁-C₈ alkyl, heteroalkyl, or cycloalkyl group which is substituted with one or more of -Me, -Et, -D, —C₂—CO₂—, —CO₂—, and —OH—. In some embodiments, R¹ is H; and R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 30 carbon atoms, and optionally having one or more heteroatoms selected from O, N, or S. In some embodiments, R¹ is H; and R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 25 carbon atoms, and optionally having one or more heteroatoms selected from O, N, or S. In some embodiments, R¹ is H; and R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 20 carbon atoms, and optionally having one or more heteroatoms selected from 0, N, or S. In some embodiments, R¹ is H; and R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 15 carbon atoms, and optionally having one or more heteroatoms selected from O, N, or S. In some embodiments, R¹ is H; and R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 10 carbon atoms, and optionally having one or more heteroatoms selected from O, N, or S.

A ninth aspect of the present disclosure is a kit comprising (i) a first conjugate comprising a first oligonucleotide coupled to a dye moiety derived from a compound having any one of Formulas (I), (IA), and (IB) (as set forth herein); and (ii) a second conjugate comprising an oligonucleotide coupled to a quencher. In some embodiments, the first conjugate has any one of Formulas (IIC) or (IID). In some embodiments, the first conjugate is directly coupled to the dye moiety. In some embodiments, the first conjugate is indirectly coupled to the dye moiety, such as through a linker (e.g., a substituted or unsubstituted linker having between 5 and about 40 carbon atoms).

A tenth aspect of the present disclosure is probe having Formula (IV):

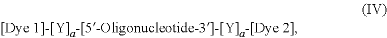

wherein
one of [Dye 1] or [Dye 2] is derived from the compound of any one of Formulas (I), (IA), and (IB); and another one of [Dye 1] or [Dye 2] is a quencher;
Oligonucleotide is an oligonucleotide having between about 5 and about 60 mer;
each Y is independently a branched or unbranched, substituted or unsubstituted, saturated or unsaturated aliphatic or aromatic group having between 2 and about 40 carbon atoms, and optionally having one or more heteroatoms selected from O, N, or S; and
a is 0, 1, or 2.

In some embodiments, the one of [Dye 1] or [Dye 2] is derived from a compound having Formula (IA):

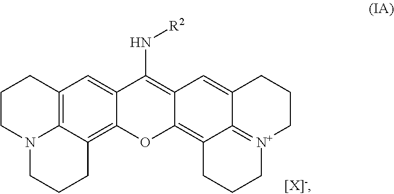

wherein R² is a C₁-C₈ branched or unbranched alkyl group substituted with one or more of -Me, -Et, —CO₂⁻, —OH, -D, or a halogen.

In some embodiments, R² is a C₁-C₆ branched or unbranched alkyl group substituted with one or more of -Me, -Et, —CO₂⁻, —OH, -D, or a halogen. In some embodiments, R² is a C₁-C₄ branched or unbranched alkyl group substituted with one or more of -Me, -Et, —CO₂⁻, —OH, -D, or a halogen. In some embodiments, a first carbon atom of R² is a primary carbon atom. In some embodiments, a first carbon atom of R² is a secondary carbon atom. In some embodiments, a first carbon atom of R² is a tertiary carbon atom. In some embodiments, R² is selected from:

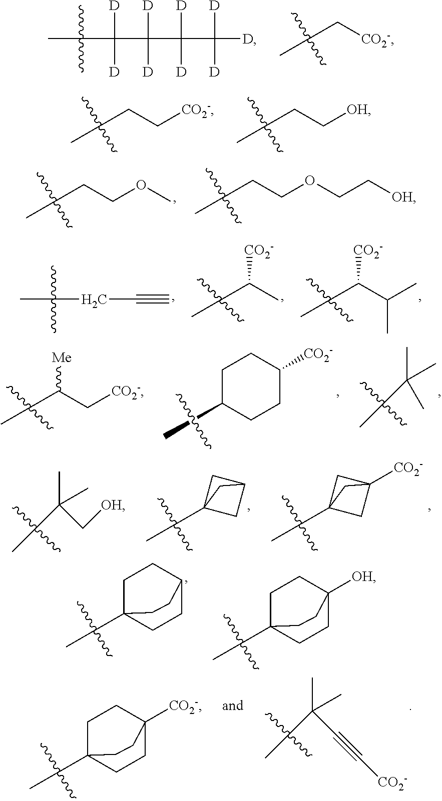

An eleventh aspect of the present disclosure is a conjugate having Formula (V):

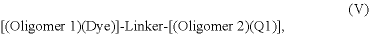

wherein
Oligomers 1 and 2 are each different and are oligonucleotides having between about 5 mer and about 30 mer;
Dye is derived from a compound having any one of Formulas (I), (IA), and (IB);
Q1 is a quencher; and
Linker is a substituted or unsubstituted aliphatic, heteroaliphatic, aromatic, or heteroaromatic group having between about 5 and about 30 carbon atoms.

In some embodiments, the Dye is derived from a compound having Formula (IA):

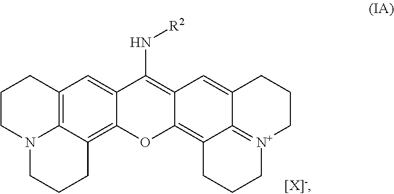

wherein R² is a C₁-C₈ branched or unbranched alkyl group substituted with one or more of -Me, -Et, —CO₂⁻, —OH, -D, or a halogen. In some embodiments, R² is a C₁-C₆ branched or unbranched alkyl group substituted with one or more of -Me, -Et, —CO₂⁻, —OH, -D, or a halogen. In some embodiments, R² is a C₁-C₄ branched or unbranched alkyl group substituted with one or more of -Me, -Et, —CO₂⁻, —OH, -D, or a halogen. In some embodiments, a first carbon atom of R² is a primary carbon atom. In some embodiments, a first carbon atom of R² is a secondary carbon atom. In some embodiments, a first carbon atom of R² is a tertiary carbon atom. In some embodiments, R² is selected from:

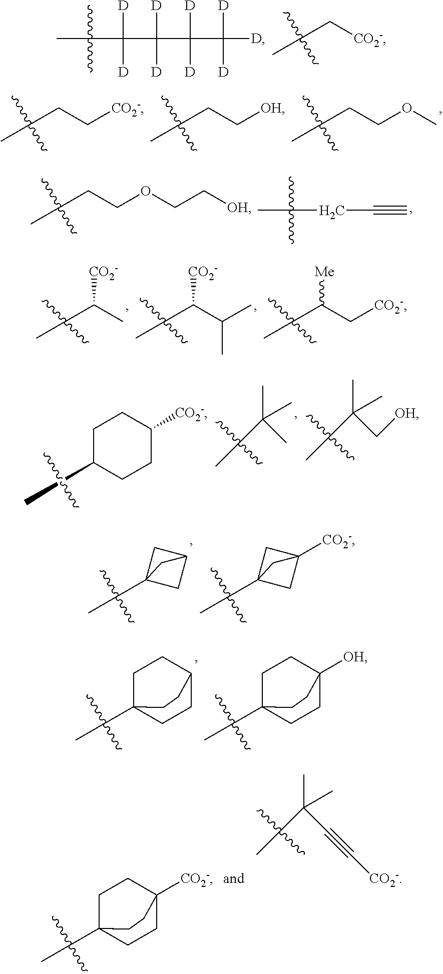

In some embodiments, the Dye has a Stokes shift of at least about 70 nm. In some embodiments, the Dye has a Stokes shift of at least about 80 nm. In some embodiments, the Dye has a Stokes shift of at least about 90 nm. In some embodiments, at least one of Oligomers 1 and 2 comprises LNA, L-LNA, or PNA. In some embodiments, the Linker is a substituted or unsubstituted aliphatic, heteroaliphatic, aromatic, or heteroaromatic group having between about 5 and about 25 carbon atoms. In some embodiments, the Linker is a substituted or unsubstituted aliphatic, heteroaliphatic, aromatic, or heteroaromatic group having between about 5 and about 20 carbon atoms. In some embodiments, the Linker is a substituted or unsubstituted aliphatic, heteroaliphatic, aromatic, or heteroaromatic group having between about 5 and about 15 carbon atoms.

A twelfth aspect of the present disclosure is a kit comprising: (i) the conjugate having any one of Formulas (IIC) and (IID); and (ii) a compound having Formula (VIII):

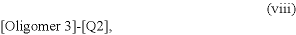

wherein
Oligomer 3 is an oligonucleotide having between 5 and 30 mer; and Q2 is a quencher.

A thirteenth aspect of the present disclosure is a FRET pair comprising a first member having Formula (VIIA) and a second member having Formula (VIIB):

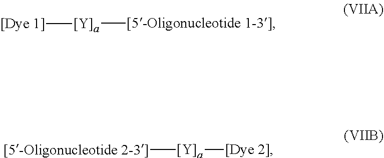

wherein
one of Dye 1 or Dye 2 are derived from a compound having of any one of Formulas (I), (IA), and (IB);
another one of Dye 1 or Dye 2 is a Quencher;
each Y is independently a branched or unbranched, linear, or cyclic, substituted or unsubstituted, saturated or unsaturated, group having between 2 and about 40 carbon atoms, and optionally having one or more heteroatoms selected from O, N, or S;
a is 0, 1, or 2; and
Oligonucleotide 1 and Oligonucleotide 2 are different.

In some embodiments, each Y is independently a branched or unbranched, linear, or cyclic, substituted or unsubstituted, saturated or unsaturated, group having between 2 and about 30 carbon atoms, and optionally having one or more heteroatoms selected from O, N, or S. In some embodiments, the one of Dye 1 or Dye 2 is derived from a compound having Formula (IA):

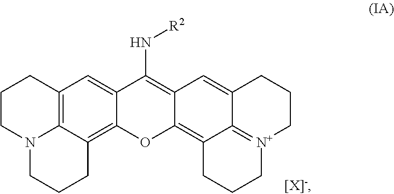

wherein R² is a C₁-C₈ branched or unbranched alkyl group substituted with one or more of -Me, -Et, —CO₂⁻, —OH, -D, or a halogen. In some embodiments, R² is a C₁-C₆ branched or unbranched alkyl group substituted with one or more of -Me, -Et, —CO₂⁻, —OH, -D, or a halogen. In some embodiments, R² is a C₁-C₄ branched or unbranched alkyl group substituted with one or more of -Me, -Et, —CO₂⁻, —OH, -D, or a halogen. In some embodiments, a first carbon atom of R² is a primary carbon atom. In some embodiments, a first carbon atom of R² is a secondary carbon atom. In some embodiments, a first carbon atom of R² is a tertiary carbon atom. In some embodiments, R² is selected from:

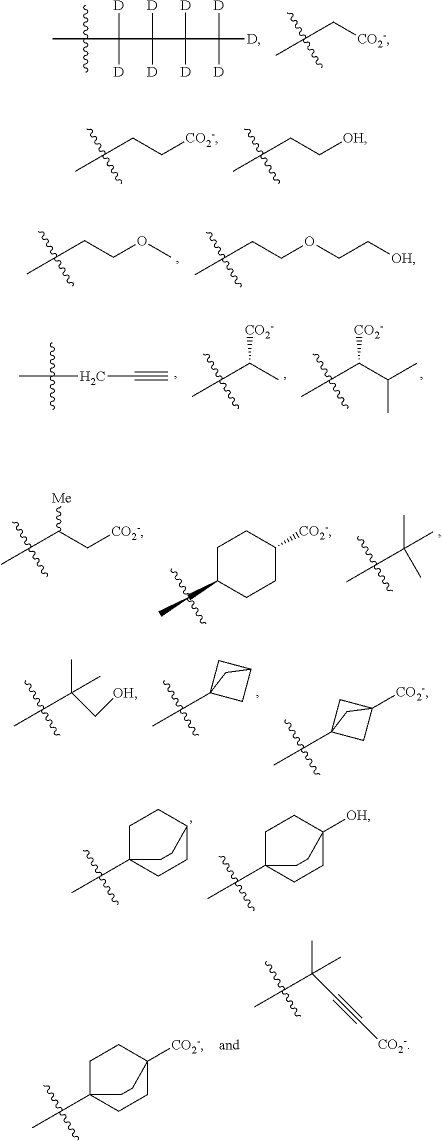

A fourteenth aspect of the present disclosure is a method for amplification and detection of a target nucleic acid in a sample comprising the steps of:
(a) contacting the sample containing the target nucleic acid in a single reaction vessel with (i) one pair of oligonucleotide primers, each oligonucleotide primer capable of hybridizing to opposite strands of a subsequence of the target nucleic acid; and (ii) an oligonucleotide probe that comprises an annealing portion and a tag portion, wherein the tag portion comprises a nucleotide sequence non-complementary to the target nucleic acid sequence, wherein the annealing portion comprises a nucleotide sequence at least partially complementary to the target nucleic acid sequence and hybridizes to a region of the subsequence of the target nucleic acid that is bounded by the pair of oligonucleotide primers, wherein the probe further comprises an interactive dual label comprising a dye derived from a compound having Formula (I), located on the tag portion and a first quencher moiety located on the annealing portion and wherein the dye is separated from the first quencher moiety by a nuclease susceptible cleavage site; and wherein prior to step (b) (recited below), the tag portion is reversibly bound in a temperature-dependent manner to a quenching oligonucleotide comprising a nucleotide sequence at least partially complementary to the tag portion of the oligonucleotide probe and binds to the tag portion by hybridization, wherein the quenching oligonucleotide comprises at least a second quencher moiety capable of quenching the dye on the tag portion when the quenching oligonucleotide is bound to the tag portion;
(b) following step (a), amplifying the target nucleic acid by polymerase chain reaction (PCR) using a nucleic acid polymerase having 5′ to 3′ nuclease activity such that during an extension step of each PCR cycle, the nuclease activity of the polymerase allows cleavage and separation of the tag portion from the first quencher moiety on the annealing portion of the probe;
(c) measuring one or more signals from the dye at a first temperature at which the quenching oligonucleotide is bound to the tag portion;
(d) measuring one or more signals from the dye at a second temperature, which is higher than the first temperature, at which the quenching oligonucleotide is not bound to the tag portion; and
(e) obtaining a calculated signal value by subtracting a median or average of the one or more signals detected at the first temperature from a median or average of the one or more signals detected at the second temperature;
whereby a calculated signal value that is higher than a threshold signal value allows determination of the presence of the target nucleic acid.

In some embodiments, the PCR amplification of step (b) is allowed to reach an endpoint beyond the log phase of amplification. In some embodiments, the tag portion comprises a modification such that it is not capable of being extended by the nucleic acid polymerase. In some embodiments, the tag portion of the oligonucleotide probe or the quenching oligonucleotide or both the tag portion and the quenching oligonucleotide contain one or more nucleotide modifications. In some embodiments, the one or more nucleotide modifications is selected from the group consisting of Locked Nucleic Acid (LNA), Peptide Nucleic Acid (PNA), Bridged Nucleic Acid (BNA), 2′-O alkyl substitution, L-enantiomeric nucleotide, and combinations thereof.

A fifteenth aspect of the present disclosure is a method of directly labeling a dye with an oligonucleotide having a terminal amine, wherein the method comprises: (i) obtaining a dye comprising a dye core and having a cyano group located at a meso position of the dye core; (ii) contacting the obtained dye in the presence of a base and a solvent with the oligonucleotide having the terminal amine, wherein a linker is positioned between the oligonucleotide and the terminal amine, wherein the linker is a C₁-C₈ alkyl, heteroalkyl, cycloalkyl, or heterocycloalkyl group which is substituted with one or more of -Me, -Et, —CO₂—, —C₂—CO₂—, -D, or a halogen.

In some embodiments, the base is selected from the group consisting of N,N-diisopropylethylamine (DIPEA), cesium carbonate, potassium carbonate, sodium carbonate, tributylamine (TBA), N,N-dicyclohexylmethylamine, 2,6-di-tert.-butylpyridine, 1,8-diazabicyclo[5.4.0]undec-7-ene (DBU), 1,5-diazabicyclo[4.3.0]non-5-ene (DBN), 1,1,3,3-tetramethylguanidine (TMG), and 2,2,6,6-tetramethylpiperidine. In some embodiments, the solvent is selected from the group consisting of dimethylsulfoxide (DMSO), sulfolane, N-butylpyrrolidone, γ-valerolactone, 6-valerolactone, N-methylpyrrolidone, N,N-dimethylformamide, sulfolane, and cyrene. In some embodiments, the oligonucleotide comprises between about 5 mer and about 60 mer. In some embodiments, the oligonucleotide comprises between about 5 mer and about 40 mer. In some embodiments, the oligonucleotide comprises between about 5 mer and about 30 mer. In some embodiments, the oligonucleotide comprises between about 5 mer and about 20 mer. In some embodiments, the oligonucleotide comprises between about 5 mer and about 15 mer. In some embodiments, the oligonucleotide comprises LNA, L-LNA, or PNA. In some embodiments, the dye having a cyano group located at the meso position of the dye core is a R800 perchlorate dye.

### BRIEF DESCRIPTION OF THE FIGURES

For a general understanding of the features of the disclosure, reference is made to the drawings. In the drawings, like reference numerals have been used throughout to identify identical elements.

FIG. 1 illustrates a method of preparing branched DNA probes to incorporate fluorescent dyes of the present disclosure.

FIG. 2 shows the optical channels and dye assignments for the Cobas x800 PCR instrument. The center wavelengths for excitation (vertical) and emission filters (horizontal) are indicated in nanometers. Fields on the diagonal correspond to optical channels that are accessible with standard fluorophores (COU, FAM, HEX, JA270, Cy5.5). Compounds R, 1a-1k (“RLS 1”) are LSS dyes that are spectrally suitable for the 435 nm/580 nm channel. Compounds 1l-1r (“RLS 2”) are spectrally suitable large stokes shift dyes for the 495 nm/580 nm channel.

FIGS. 3A, 3B, and 3C summarize analytical data for the R800 dye (upper panels) and compound R (lower panels). Shown are chromatograms, mass spectra in positive ion mode, and absorption spectra.

FIG. 4A shows the chromatogram at the absorption maximum for compound R.

FIG. 4B shows the absorption and fluorescence emission spectra for compound R.

FIG. 4C shows the mass spectrum in positive ion mode for compound R.

FIG. 5A shows the chromatogram at the absorption maximum for compound 1a.

FIG. 5B shows the absorption and fluorescence emission spectra for compound 1a.

FIG. 5C shows the mass spectrum in positive ion mode for compound 1a.

FIG. 6 shows the mass spectrum in positive ion mode for compound 1b.

FIG. 7A shows the chromatogram at the absorption maximum for compound 1c.

FIG. 7B shows the absorption and fluorescence emission spectra for compound 1c.

FIG. 7C shows the mass spectrum in positive ion mode for compound 1c.

FIG. 8A shows the chromatogram at the absorption maximum for compound 1d.

FIG. 8B shows the absorption and fluorescence emission spectra for compound 1d.

FIG. 8C shows the mass spectrum in positive ion mode for compound 1d.

FIG. 9A shows the chromatogram at the absorption maximum for compound 1e.

FIG. 9B shows the absorption and fluorescence emission spectra for compound 1e.

FIG. 9C shows the mass spectrum in positive ion mode for compound 1e.

FIG. 10A shows the chromatogram at the absorption maximum for compound 1f.

FIG. 10B shows the absorption and fluorescence emission spectra for compound 1f.

FIG. 10C shows the mass spectrum in positive ion mode for compound 1f.

FIG. 11A shows the chromatogram at the absorption maximum for compound 1g.

FIG. 11B shows the absorption and fluorescence emission spectra for compound 1g.

FIG. 11C shows the mass spectrum in positive ion mode for compound 1g.

FIG. 12A shows the chromatogram at the absorption maximum for compound 1h.

FIG. 12B shows the absorption and fluorescence emission spectra for compound 1h.

FIG. 12C shows the mass spectrum in positive ion mode for compound 1h.

FIG. 13A shows the chromatogram at the absorption maximum for compound 1i.

FIG. 13B shows the absorption and fluorescence emission spectra for compound 1i.

FIG. 13C shows the mass spectrum in positive ion mode for compound 1i.

FIG. 14A shows the chromatogram at the absorption maximum for compound 1j.

FIG. 14B shows the absorption and fluorescence emission spectra for compound 1j.

FIG. 14C shows the mass spectrum in positive ion mode for compound 1j.

FIG. 15A shows the chromatogram at the absorption maximum for compound 1k.

FIG. 15B shows the absorption and fluorescence emission spectra for compound 1k.

FIG. 15C shows the mass spectrum in positive ion mode for compound 1k.

FIG. 16A shows the chromatogram at the absorption maximum for compound 1l.

FIG. 16B shows the absorption and fluorescence emission spectra for compound 1l.

FIG. 16C shows the mass spectrum in positive ion mode for compound 1l.

FIG. 17A shows the chromatogram at the absorption maximum for compound 1m.

FIG. 17B shows the absorption and fluorescence emission spectra for compound 1m.

FIG. 17C shows the mass spectrum in positive ion mode for compound 1m.

FIG. 18A shows the chromatogram at the absorption maximum for compound 1n.

FIG. 18B shows the absorption and fluorescence emission spectra for compound 1n.

FIG. 18C shows the mass spectrum in positive ion mode for compound 1n.

FIG. 19A shows the chromatogram at the absorption maximum for compound 1o.

FIG. 19B shows the absorption and fluorescence emission spectra for compound 1o.

FIG. 19C shows the mass spectrum in positive ion mode for compound 1o.

FIG. 20A shows the chromatogram at the absorption maximum for compound 1p.

FIG. 20B shows the absorption and fluorescence emission spectra for compound 1p.

FIG. 20C shows the mass spectrum in positive ion mode for compound 1p.

FIG. 21A shows the chromatogram at the absorption maximum for compound 1q.

FIG. 21B shows the absorption and fluorescence emission spectra for compound 1q.

FIG. 21C shows the mass spectrum in positive ion mode for compound 1q.

FIG. 22A shows the chromatogram at the absorption maximum for compound 1r.

FIG. 22B shows the absorption and fluorescence emission spectra for compound 1r.

FIG. 22C shows the mass spectrum in positive ion mode for compound 1r.

FIG. 23A shows the chromatogram at the absorption maximum for compound 1s.

FIG. 23B shows the absorption and fluorescence emission spectra for compound 1s.

FIG. 23C shows the mass spectrum in positive ion mode for compound 1s.

FIG. 24 shows the thermostability of fluorescence for compounds R (hollow line), 1i (solid line), 1k (dotted line), 1j (dashed and dotted line), and in (dashed line). Each sample was excited at the respective absorption maximum and the fluorescence signal at the emission maximum was plotted as a function of temperature. For simplicity, the data has not been normalized to concentration.

FIG. 25 shows direct labeling of a BHQ-2 and amino-modified 36mer DNA with R800 dye. DNA labeling was achieved without the need for a carboxylic acid functionality since the dye-linker moiety stems from the DNA amino-modifier. All chromatograms were obtained with the same mobile phase gradient. The first chromatogram shows the retention time of the pre-purified DNA starting material. The second chromatogram shows the retention time of the R800 dye. The third chromatogram shows separation of the labeling reaction with the labeled DNA-dye conjugate. The fourth panel shows the purified DNA-dye conjugate. An overlay of absorption spectra of the DNA before and after the labeling reaction is shown in the upper right panel. The lower right panel shows the deconvoluted mass spectrum in negative ion mode of the DNA-dye conjugate.

FIG. 26A shows chromatograms of three different 5′-azido modified DNA sequences of different length (15mer, 16mer, and 37mer) that have been labeled with R800 dye. The DNA-dye conjugates have been obtained through direct labeling, i.e., without the need for active ester or click-chemistry functionalities. With this approach the amino-linker of the DNA becomes an integral part of the dye structure. All chromatograms were obtained with the same mobile phase gradient.

FIG. 26B shows the absorption spectra (left) and deconvoluted mass spectra (right) in negative ion mode for the DNA sequences in FIG. 26A.

FIG. 27 shows a 5′-azido 15mer DNA that was labeled with compound 1j. The DNA-1j conjugate was obtained through amide-bond formation between the amino-functionality at the 5′-penultimate position of the DNA and the in situ generated active ester of compound 1j. The chromatogram shows the retention times of the DNA-1j conjugate. Because compound 1j is a racemic mixture of enantiomers, the labeled DNA is a mixture of diastereomers and separates as double peak. The absorption spectra for the left and right peak are shown in the two middle panels. The bottom panel shows the deconvoluted mass spectrum in negative ion mode of the DNA-1j conjugate.

FIG. 28 shows PCR growth curves (duplicates) of a TaqMan® DNA probe with a LSS dye that has been prepared in Example 6 (solid lines). The fluorescence was detected in the LSS channel (FIG. 2, channel RLS 1, 435 nm/580 nm). PCR growth curves were also obtained with the same TaqMan® sequence that was labeled with Cy5.5 dye (dashed lines). The fluorescence was detected in the Cy5.5 channel (FIG. 2, 580/700 nm) and overlaid in the graph for comparison.

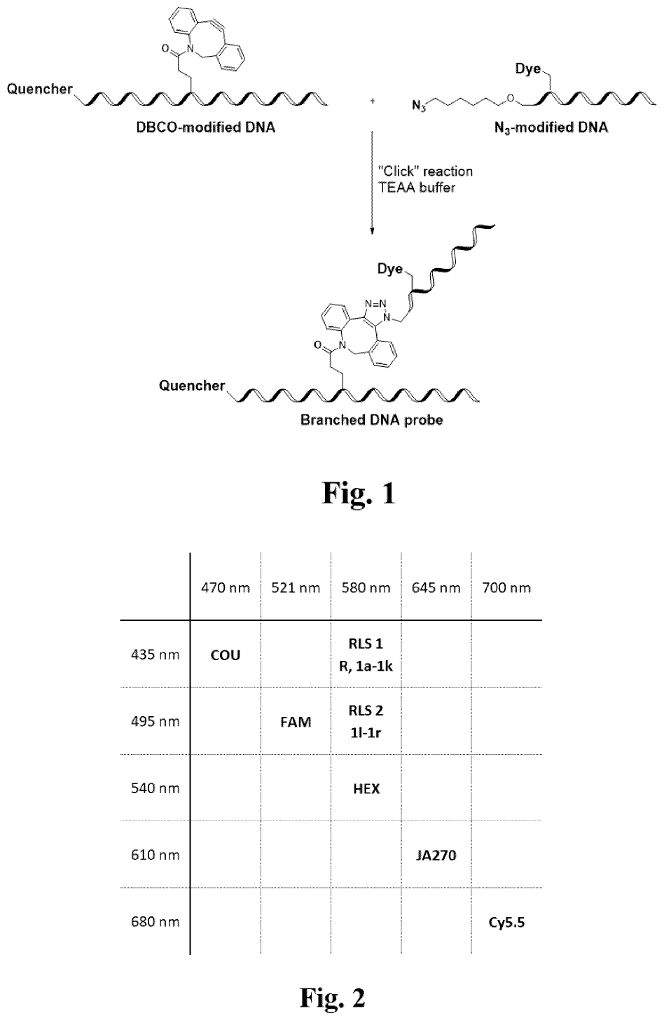

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the symbol “ [symbol] ” refers to a location in which a moiety is bonded to another moiety.

As used herein, the term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl group substituted cycloalkyl groups, and cycloalkyl group substituted alkyl groups. The term alkyl further includes alkyl groups, which may include one or more heteroatoms, such as oxygen, nitrogen, sulfur or phosphorous atoms replacing one or more carbons of the hydrocarbon backbone. In certain embodiments, a straight chain or branched chain alkyl has 8 or fewer carbon atoms in its backbone (e.g., C₁-C₈ for straight chain, C₁-C₈ for branched chain). Moreover, the term alkyl includes both “unsubstituted alkyls” and “substituted alkyls,” the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “amine reactive group refers to a reagent or a group that may form a covalent bond with an amine group or another molecule.

As used herein, the term “antibody” refers to immunoglobulins or immunoglobulin-like molecules, including by way of example and without limitation, IgA, IgD, IgE, IgG and IgM, combinations thereof, and similar molecules produced during an immune response in any vertebrate, (e.g., in mammals such as humans, goats, rabbits and mice) and antibody fragments that specifically bind to a molecule of interest (or a group of highly similar molecules of interest) to the substantial exclusion of binding to other molecules. Antibody further refers to a polypeptide ligand comprising at least a light chain or heavy chain immunoglobulin variable region which specifically recognizes and binds an epitope of an antigen. Antibodies may be composed of a heavy and a light chain, each of which has a variable region, termed the variable heavy (VH) region and the variable light (VL) region. Together, the VH region and the VL region are responsible for binding the antigen recognized by the antibody. The term antibody also includes intact immunoglobulins and the variants and portions of them well known in the art.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl or aryl group, or the total number of carbon atoms and heteroatoms in a heteroalkyl, heterocyclyl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂, CH₃CH₂CH(CH₃)— and (CH₃)₃C—.

As used herein, the term “carbonyl reactive group refers to a reagent or a group that may form a covalent bond with a carbonyl group or another molecule.

As used herein, the term “click chemistry” refers to a chemical philosophy, independently defined by the groups of Sharpless and Meldal, that describes chemistry tailored to generate substances quickly and reliably by joining small units together. “Click chemistry” has been applied to a collection of reliable and self-directed organic reactions (Kolb, H. C.; Finn, M. G.; Sharpless, K. B. Angew). Chem. Int. Ed. 2001, 40, 2004-2021). For example, the identification of the copper catalyzed azide-alkyne [3+2]cycloaddition as a reliable molecular connection in water (Rostovtsev, V. V.; et al. Angew. Chem. Int. Ed. 2002, 41, 2596-2599) has been used to augment several types of investigations of biomolecular interactions (Wang, Q.; et al. J. Am. Chem. Soc. 2003, 125, 3192-3193; Speers, A. E.; et al. J. Am. Chem. Soc. 2003, 125, 4686-4687; Link, A. J.; Tirrell, D. A. J. Am. Chem. Soc. 2003, 125, 11164-11165; Deiters, A.; et al. J. Am. Chem. Soc. 2003, 125, 11782-11783). In addition, applications to organic synthesis (Lee, L. V.; et al. J. Am. Chem. Soc. 2003, 125, 9588-9589), drug discovery (Kolb, H. C.; Sharpless, K. B. Drug Disc. Today 2003, 8, 1128-1137; Lewis, W. G.; et al. Angew. Chem. Int. Ed. 2002, 41, 1053-1057), and the functionalization of surfaces (Meng, J.-C.; et al. Angew. Chem. Int. Ed. 2004, 43, 1255-1260; Fazio, F.; et al. J. Am. Chem. Soc. 2002, 124, 14397-14402; Collman, J. P.; et al. Langmuir 2004, ASAP, in press; Lummerstorfer, T.; Hoffmann, H. J. Phys. Chem. B 2004, in press) have also appeared. Generally, click chemistry encourages reactions that have modular applications that are wide in scope, that have a high chemical yield, that generate inoffensive by-products, that are chemospecific, that require simple reaction conditions, that use readily available starting materials and reagents, that are solvent free or use benign solvents (such as water), that lead to easy product isolation, that have a large thermodynamic driving force to favor a reaction with a single reaction product, and that have a high atom economy. While certain of the general criteria can be subjective in nature, and not all criteria need to be met.

As used herein, the term “conjugate” refers to two or more molecules or moieties (including macromolecules or supra-molecular molecules) that are covalently linked into a larger construct. In some embodiments, a conjugate includes one or more biomolecules (such as peptides, proteins, enzymes, sugars, polysaccharides, lipids, glycoproteins, and lipoproteins) covalently linked to one or more other molecules moieties.

As used herein, the terms “couple,” “coupled,” or “coupling” refers to the joining, bonding (e.g., covalent bonding), or linking of one molecule or atom to another molecule or atom.

As used herein, the term “derivative” is used in accordance with its plain ordinary meaning within chemistry and biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the term “heteroatom” is meant to include boron (B), oxygen (O), nitrogen (N), sulfur (S), phosphorus (P), and silicon (Si). As noted herein, in some embodiments, a “heterocyclic ring” may comprise one or more heteroatoms. In other embodiments, an aliphatic group may comprise or be substituted by one or more heteroatoms.

As used herein, the term “oligonucleotide” refers to linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target nucleic acid. Usually, monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG,” it will be understood that the nucleotides are in 5′-3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually, oligonucleotides comprise the four natural deoxynucleotides; however, they may also comprise ribonucleosides or non-natural nucleotide analogs, as noted above. Where an enzyme has specific oligonucleotide or polynucleotide substrate requirements for activity, e.g., single stranded DNA, RNA/DNA duplex, or the like, then selection of the appropriate composition for the oligonucleotide or polynucleotide substrates is well within the knowledge of one of ordinary skill. As used herein, the term “phosphoramidite” refers to a trivalent phosphorus group typically used in oligonucleotide synthesis. Detailed descriptions of the chemistry used to form oligonucleotides by the phosphoramidite method are provided in Caruthers et al., U.S. Pat. Nos. 4,458,066 and 4,415,732; Caruthers et al., Genetic Engineering, 4:1-17 (1982); Users Manual Model 392 and 394 Polynucleotide Synthesizers, pages 6-1 through 6-22, Applied Biosystems, Part No. 901237 (1991), each of which are incorporated by reference in their entirety.

As used herein, the term “primary antibody” refers to an antibody which binds specifically to the target protein antigen in a tissue sample. A primary antibody is generally the first antibody used in an immunohistochemical procedure.

As used herein, the term “protecting group” refers to a moiety that when attached to a reactive group in a molecule masks, reduces or prevents that reactivity. A “protected” molecule has one or more reactive groups (e.g., hydroxyl, amino, thiol, etc.) protected by protecting groups. Examples of protecting groups can be found in T. W. Greene and P. G. M. Wuts, Protective Groups in Organic Synthesis, 3rd edition, John Wiley & Sons, New York, 1999, Harrison and Harrison et al. Compendium of Synthetic Organic Methods, Vols. 1-8 (John Wiley and Sons, 1971-1996), and “Protection of Nucleosides for Oligonucleotide Synthesis,” Current Protocols in Nucleic Acid Chemistry, ed. by Boyle, A. L., John Wiley & Sons, Inc., 2000, New York, N.Y., all of which are incorporated herein by reference in their entirety.

As used herein, the terms “reactive group” or “reactive functional group” refer to a functional group that are capable of chemically associating with, interacting with, hybridizing with, hydrogen bonding with, or coupling with a functional group of a different moiety. In some embodiments, a “reaction” between two reactive groups or two reactive functional groups may mean that a covalent linkage is formed between two reactive groups or two reactive functional groups; or may mean that the two reactive groups or two reactive functional groups associate with each other, interact with each other, hybridize to each other, hydrogen bond with each other, etc. In some embodiments, the “reaction” thus includes binding events, such as the binding of a hapten with an anti-hapten antibody, or a guest molecule associating with a supramolecular host molecule.

As used herein, the term “secondary antibody” herein refers to an antibody which binds specifically to a primary antibody, thereby forming a bridge between the primary antibody and a subsequent reagent (e.g., a label, an enzyme, etc.), if any. The secondary antibody is generally the second antibody used in an immunohistochemical procedure.

As used herein, the term “specific binding entity” refers to a member of a specific-binding pair. Specific binding pairs are pairs of molecules that are characterized in that they bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins (for example, antibodies, lectins, avidins such as streptavidins, and protein A). Specific binding moieties can also include the molecules (or portions thereof) that are specifically bound by such specific binding proteins.

As used herein, the term “stokes shift” refers to the difference (in wavelength or frequency unites) between positions of the band maxima of the absorption and emission spectra (fluorescence and Raman being two examples) of the same electronic transition. When a system (be it a molecule or atom) absorbs a photon, it gains energy and enters an excited state. One way for the system to relax is to emit a photon, thus losing its energy.

It is believed that the vast majority of small-molecule fluorophores exhibit Stokes shifts in the order of 10-25 nm. Fluorophores with significantly larger Stokes shifts are loosely referred to as “large Stokes shift” (LSS) dyes, “high Stokes shift” dyes, or “MegaStokes” dyes. Two photophysical mechanisms are discussed in the literature to explain the occurrence of large Stokes shifts. The molecular geometry-type mechanism is based on the conformational relaxation of the fluorophore in the excited state and the resulting rearrangement of the surrounding solvent dipoles. The Stokes shift grows with the increasing difference between the (equilibrated) molecular geometries and dipole moments in the ground and excited states. Large Stokes shift fluorescence for the electronic-type mechanism is ascribed to intramolecular charge transfer (ICT) in the excited state. A common problem of fluorophores with small Stokes shift is internal quenching of fluorescence. Such self-quenching is caused by spectral overlap of excitation and emission, and especially prevalent at high fluorophore concentrations. LSS dyes have better separated spectral bands, which minimizes the reabsorption of photons. There is a non-zero probability for excitation of fluorophores outside of their major excitation peak. In consequence, fluorescence from one dye inevitably contributes to the total light detected in multiple emission channels. This spectral “crosstalk” or “bleed-through” can to some extent be compensated for computationally, by using predetermined correction factors. Additionally, scattering of excitation light adds to the background fluorescence in neighboring channels. LSS dyes allow to reduce or even avoid crosstalk and scattering from other fluorophores. LSS dyes are especially useful in experimental settings where many fluorophores generate a strong background signal. Large spectral separation as for LSS dyes allows for more effective filtering of the excitation light, thereby enhancing the sensitivity of target detection. LSS dyes give access to fluorescence data from previously inaccessible optical channels. Facilitated by broad spectral separation, and when used in combination with standard fluorophores, LSS dyes allow to increase the multiplexing capabilities of fluorometric PCR devices. This way LSS labels allow the implementation of additional channels to established four- to six-color instruments. In principle 21 channels can be composed from the filter combinations of a six-color instrument. However, in practice the number of channels is limited by the commercial availability of LSS dyes with sufficiently large Stokes shift. Based on a Stokes shift of 150 nm for LSS dyes that are currently available on the market, nine additional channels can be implemented. The channels for standard dyes are highlighted in light grey, whereas dark grey indicates channels for which suitable LSS dyes are currently not available. Instead, resonance electron transfer (RET) probes produce large “virtual” Stokes shift and can also be used to access these channels.

Whenever a group or moiety is described as being “substituted” or “optionally substituted” that group may be unsubstituted or substituted with one or more of the indicated substituents. Likewise, when a group is described as being “substituted or unsubstituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, cyanate, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, ether, amino (e.g. a mono-substituted amino group or a di-substituted amino group), and protected derivatives thereof. Any of the above groups may include one or more heteroatoms, including O, N, or S. For example, where a moiety is substituted with an alkyl group, that alkyl group may comprise a heteroatom selected from O, N, or S (e.g. —(CH₂—CH₂—O—CH₂—CH₂)—).

As used herein, the term “thiol reactive group” refers to a reagent or a group that may form a covalent bond with a thiol group or another molecule.

### Overview

The present disclosure is directed to dyes, such as dyes having a larges Stokes shift. The present disclosure is also directed to conjugates and probes including one or more of the disclosed dyes. The present disclosure also provides kits including one or more of the disclosed dyes; or one or more conjugates including one or more of the disclosed dyes. The dyes of the present disclosure may be used with any fluorescence-based PCR platform with compatible optical filters. Conjugates including or derived from one of the dyes disclosed herein are also compatible with PCR with TAGS (Temperature assisted generation of signal) technology, provided that the dye exhibits stable fluorescence at temperatures up to 100° C. (see U.S. Pat. Nos. 11,028,433, 11,034,997, and 11,345,958; and in United States Patent Publication No. 2021/0269857, the disclosures of which are hereby incorporated by reference herein in their entireties).

A common issue with fluorophores having a “small” Stokes shift is internal quenching of fluorescence. Such self-quenching is caused by spectral overlap of excitation and emission, and especially prevalent at high fluorophore concentrations. “Large” stokes shift dyes, such as those of the present disclosure, generally have better separated spectral bands, which minimizes the reabsorption of photons.

It is believed that there is a non-zero probability for excitation of fluorophores outside of their major excitation peak. As a consequence, the fluorescence from a dye inevitably contributes to the total light detected in multiple emission channels. This spectral “crosstalk” or “bleed-through” can, to some extent, be compensated for computationally, by using predetermined correction factors.

Additionally, scattering of excitation light adds to the background fluorescence in neighboring channels. “Large” stokes shift dyes, such as those of the present disclosure, allow for a reduction in crosstalk and scattering from other fluorophores. “Large” stokes shift dyes, such as those of the present disclosure, are especially useful in experimental settings where many fluorophores generate a strong background signal. The large spectral separation of “large” stokes shift dyes, such as those of the present disclosure, is believed to allow for more effective filtering of the excitation light, thereby enhancing the sensitivity of target detection.

“Large” stokes shift dyes, such as those of the present disclosure, also provide access to fluorescence data from previously inaccessible optical channels. Facilitated by their broad spectral separation and when used in combination with standard fluorophores, “Large” stokes shift dyes, such as those of the present disclosure, allow for an increase in the multiplexing capabilities of fluorometric PCR devices by adding further channels to established four- to six-color instruments. In principle 21 channels are available from the filter combinations of a six-color instrument. In practice, however, the number of channels is limited by the commercial availability of dyes with suitable spectral properties and sufficiently large Stokes shift.

### Dyes or Dye Precursors

The present disclosure provides compounds having Formula (I):

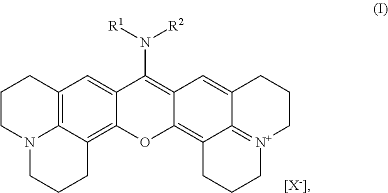

wherein
R¹ is H or a protecting group;
R² is a C₁-C₈ branched or unbranched alkyl, branched or unbranched heteroalkyl, or cycloalkyl group which is substituted with one or more of -Me, -Et, —CO₂⁻, —CO₂-(thiol reactive group), —CO₂-(amine reactive group), —CO₂-(carboxy reactive group), —C₂—CO₂⁻, —C₂—CO₂-(thiol reactive group), —C₂—CO₂-(amine reactive group), —C₂—CO₂-(carboxy reactive group), —OH, -phosphoramidite, —O-phosphoramidite, -D, a halogen, or a group capable of participating in a “click chemistry” reaction; and
[X]⁻ is a counter anion, provided when R₂ has a negative charge, [X]⁻ is not present.

In some embodiments, [X] is chloride, bromide, iodide, sulfate, benzene sulfonate, p-toluenesulfonate, p-bromobenzenesulfonate, methanesulfonate, trifluoromethanesulfonate, phosphate, perchlorate, tetrafluoroborate, hexafluorophosphate, tetraphenylboride, nitrate and anions of aromatic or aliphatic carboxylic acids. In some embodiments, R¹ is H. In other embodiments, R¹ is 9-fluorenylmethyl carbamate, t-Butyl carbamate, benzyl carbamate, acetamide, trifluoroacetamide, benzylamine, triphenylmethylamine, monomethoxytrityl (MMT), DMS, and p-toluenesulfonamide. In some embodiments, the first carbon atom of R² is a primary carbon atom. In other embodiments, the first carbon atom of R² is a secondary carbon atom. In yet other embodiments, the first carbon atom of R² is a tertiary carbon atom. In some embodiments, R² includes no substitutions. In other embodiments, R² includes one substitution. In other embodiments, R² includes two or more substitutions. In some embodiments, R² includes a heteroalkyl group having a heteroatom selected from O, N, or S. In some embodiments, R² includes a heteroalkyl group having an O heteroatom. In some embodiments, the group capable of participating in a “click chemistry reaction” is selected from a bicyclo[6.1.0]nonyne) group (“BCN”), dibenzocyclooctyne (“DBCO”), alkene, trans-cycloctene (“TCO”), maleimide, an aldehyde, a ketone, an azide, a tetrazine, a thiol, a 1,3-nitrone, a hydrazine, or a hydroxylamine. In some embodiments, R¹ is H; and R² is one of BCN, DBCO, TCO, an azide, tetrazine, or a maleimide. In some embodiments, the compounds of Formula (I) terminate in one of a thiol-reactive group, an amine-reactive group, or a carboxy-reactive group. In some embodiments, the thiol-reactive group is selected from a haloacetyl, a maleimide, an iodoacetamide, an aziridine, an acryloyl, an arylating agent, a vinylsulfone, a methanethiosulfonate, a pyridyl disulfide, a TNB-thiol and a disulfide reducing agent. In some embodiments, the thiol-reactive group can comprise a maleimide. In some embodiments, the thiol-reactive group can comprise a haloacetyl. In some embodiments, the thiol-reactive group can comprise an aziridine. In some embodiments, the thiol-reactive group can comprise an acryloyl. In some embodiments, the thiol-reactive group can comprise an arylating agent. In some embodiments, the thiol-reactive group can comprise a vinylsulfone. In some embodiments, the thiol-reactive group can comprise a pyridyl disulfide. In some embodiments, the thiol-reactive group can comprise a TNB-thiol. In some embodiments, the thiol-reactive group can comprise a disulfide reducing agent. In some embodiments, the amine-reactive group is selected from an NHS ester (e.g., NHS, sulfo-NHS, N-hydroxy-5-norbornene-2,3-dicarboxylic acid imide), an isothiocyanate, an acyl azide, a sulfonyl chloride, a sulfodichlorophenol, pentafluorophenol, tetrafluorophenol, 4-sulfo-2,3,5,6-tetrafluorophenyl, an aldehyde, a glyoxal, an epoxide, an oxirane, a carbonate, an aryl halide, a fluorophenol ester, a sulfochlorophenol, an uranium, a carbodiimide, a phthalimide, a benzotriazole, an imidoester, an anhydride and the like. In some embodiments, the carbonyl-reactive group is hydrazine, a hydrazine derivative, or an amine. In some embodiments, R² is C₁-C₈ branched or unbranched alkyl, branched or unbranched heteroalkyl, or cycloalkyl group which is substituted with a —CO₂— maleimide. In some embodiments, R² is C₁-C₆ branched or unbranched alkyl, branched or unbranched heteroalkyl, or cycloalkyl group which is substituted with a —CO₂-maleimide. In some embodiments, R² is C₁-C₄ branched or unbranched alkyl, branched or unbranched heteroalkyl, or cycloalkyl group which is substituted with a —CO₂-maleimide. In some embodiments, R² is C₁-C₈ branched or unbranched alkyl, branched or unbranched heteroalkyl, or cycloalkyl group which is substituted with a —C₂—CO₂-maleimide. In some embodiments, R² is C₁-C₆ branched or unbranched alkyl, branched or unbranched heteroalkyl, or cycloalkyl group which is substituted with a —C₂—CO₂-maleimide. In some embodiments, R² is C₁-C₄ branched or unbranched alkyl, branched or unbranched heteroalkyl, or cycloalkyl group which is substituted with a —C₂—CO₂— maleimide. In some embodiments, R² is C₁-C₈ branched or unbranched alkyl, branched or unbranched heteroalkyl, or cycloalkyl group which is substituted with a —CO₂—NHS ester. In some embodiments, R² is C₁-C₆ branched or unbranched alkyl, branched or unbranched heteroalkyl, or cycloalkyl group which is substituted with a —CO₂—NHS ester. In some embodiments, R² is C₁-C₄ branched or unbranched alkyl, branched or unbranched heteroalkyl, or cycloalkyl group which is substituted with a —CO₂—NHS ester. In some embodiments, R² is C₁-C₈ branched or unbranched alkyl, branched or unbranched heteroalkyl, or cycloalkyl group which is substituted with a —C₂—CO₂—NHS ester. In some embodiments, R² is C₁-C₆ branched or unbranched alkyl, branched or unbranched heteroalkyl, or cycloalkyl group which is substituted with a —C₂—CO₂—NHS ester. In some embodiments, R² is C₁-C₄ branched or unbranched alkyl, branched or unbranched heteroalkyl, or cycloalkyl group which is substituted with a —C₂—CO₂—NHS ester. In some embodiments, R² is C₁-C₈ branched or unbranched alkyl, branched or unbranched heteroalkyl, or cycloalkyl group which is substituted with a —CO₂-hydrazine. In some embodiments, R² is C₁-C₆ branched or unbranched alkyl, branched or unbranched heteroalkyl, or cycloalkyl group which is substituted with a —CO₂— hydrazine. In some embodiments, R² is C₁-C₄ branched or unbranched alkyl, branched or unbranched heteroalkyl, or cycloalkyl group which is substituted with a —CO₂-hydrazine. In some embodiments, R² is C₁-C₈ branched or unbranched alkyl, branched or unbranched heteroalkyl, or cycloalkyl group which is substituted with a —C₂—CO₂— hydrazine. In some embodiments, R² is C₁-C₆ branched or unbranched alkyl, branched or unbranched heteroalkyl, or cycloalkyl group which is substituted with a —C₂—CO₂— hydrazine. In some embodiments, R² is C₁-C₄ branched or unbranched alkyl, branched or unbranched heteroalkyl, or cycloalkyl group which is substituted with a —C₂—CO₂— hydrazine. In some embodiments, R² is a C₁-C₈ branched or unbranched alkyl group substituted with one or more of -Me, -Et, —CO₂⁻, —OH, -phosphoramidite, —O-phosphoramidite, -D, or a group capable of participating in a “click chemistry” reaction. In some embodiments, R² is a C₁-C₈ branched or unbranched alkyl group substituted with one or more of -Me, -Et, —CO₂⁻, —OH, -D, halogen, or a group capable of participating in a “click chemistry” reaction. In some embodiments, R² is a C₁-C₈ branched or unbranched alkyl group substituted with one or more of -Me, -Et, —CO₂⁻, —OH, -D, or halogen. In some embodiments, R₂ is a C₁-C₆ branched or unbranched alkyl group substituted with BCN, DBCO or TCO. In some embodiments, R² is a C₁-C₆ branched or unbranched alkyl, branched or unbranched heteroalkyl, or cycloalkyl group which is substituted with one or more of -Me, -Et, —CO₂⁻, —CO₂-(thiol reactive group), —CO₂-(amine reactive group), —CO₂-(carboxy reactive group), —C₂—CO₂⁻, —C₂—CO₂-(thiol reactive group), —C₂—CO₂-(amine reactive group), —C₂—CO₂-(carboxy reactive group), —OH, -phosphoramidite, —O-phosphoramidite, -D, halogen, or a group capable of participating in a “click chemistry” reaction. In some embodiments, R² is a C₁-C₆ branched or unbranched alkyl group substituted with one or more of -Me, -Et, —CO₂⁻, —OH, -phosphoramidite, —O-phosphoramidite, -D, or a group capable of participating in a “click chemistry” reaction. In some embodiments, R² is a C₁-C₆ branched or unbranched alkyl group substituted with one or more of -Me, -Et, —CO₂⁻, —OH, -D, a halogen, or a group capable of participating in a “click chemistry” reaction. In some embodiments, R² is a C₁-C₆ branched or unbranched alkyl group substituted with one or more of -Me, -Et, —CO₂⁻, —OH, -D, or halogen. In some embodiments, R² is a C₁-C₆ branched or unbranched alkyl group substituted with BCN, DBCO or TCO. In some embodiments, R² is a C₁-C₄ branched or unbranched alkyl, branched or unbranched heteroalkyl, or cycloalkyl group which is substituted with one or more of -Me, -Et, —CO₂⁻, —CO₂-(thiol reactive group), —CO₂-(amine reactive group), —CO₂-(carboxy reactive group), —C₂—CO₂⁻, —C₂—CO₂-(thiol reactive group), —C₂—CO₂-(amine reactive group), —C₂—CO₂-(carboxy reactive group), —OH, -phosphoramidite, —O-phosphoramidite, -D, halogen, or a group capable of participating in a “click chemistry” reaction. In some embodiments, R² is a C₁-C₄ branched or unbranched alkyl group substituted with one or more of -Me, -Et, —CO₂⁻, —OH, -phosphoramidite, —O-phosphoramidite, -D, or a group capable of participating in a “click chemistry” reaction. In some embodiments, R² is a C₁-C₄ branched or unbranched alkyl group substituted with one or more of -Me, -Et, —CO₂⁻, —OH, -D, halogen, or a group capable of participating in a “click chemistry” reaction. In some embodiments, R² is a C₁-C₄ branched or unbranched alkyl group substituted with one or more of -Me, -Et, —CO₂⁻, —OH, -D, or a halogen. In some embodiments, R² is a C₁-C₄ branched or unbranched alkyl group substituted with BCN, DBCO or TCO. In some embodiments, R² is selected from:

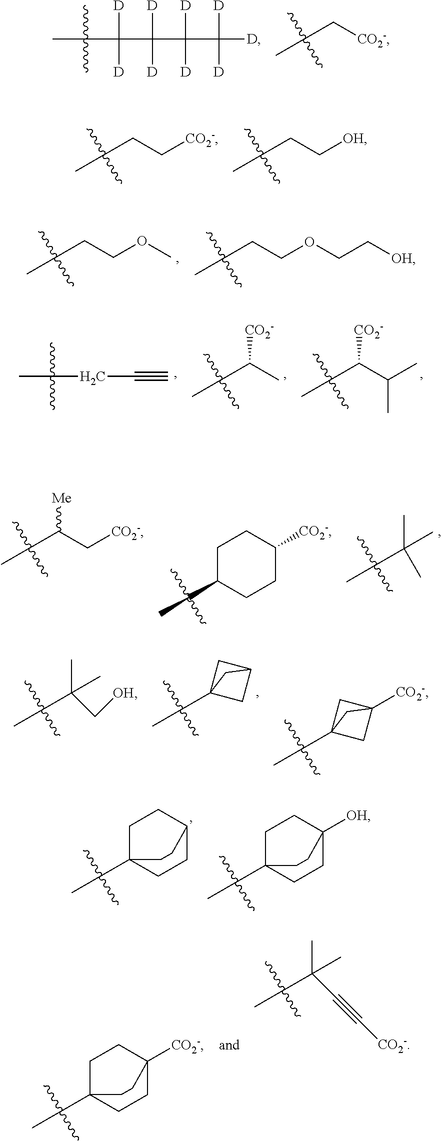

Examples of compounds of Formula (I) include, but are not limited to, the following:

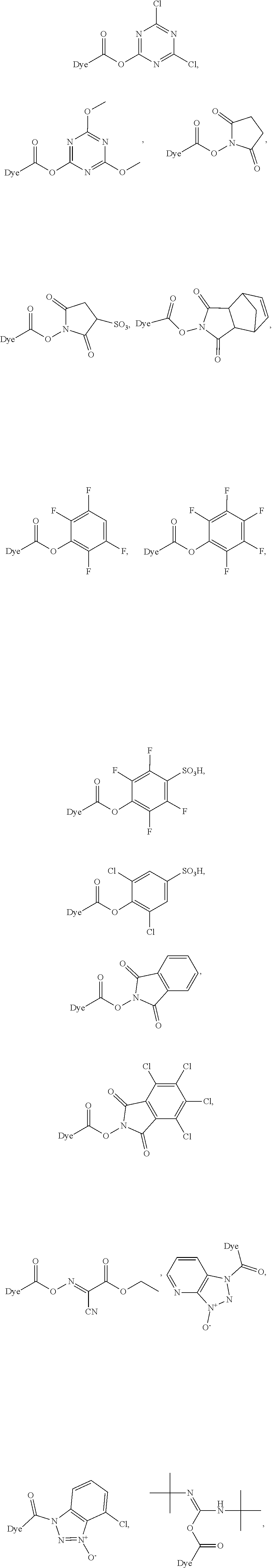

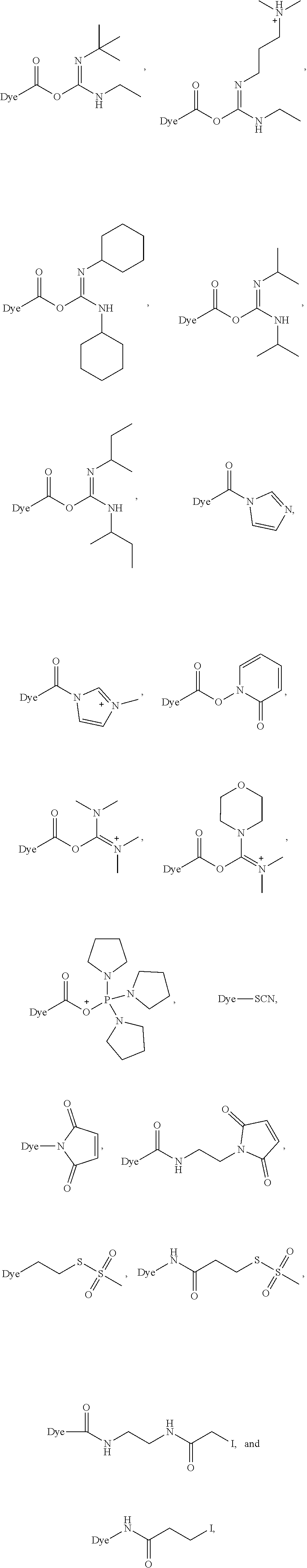

where “Dye” is

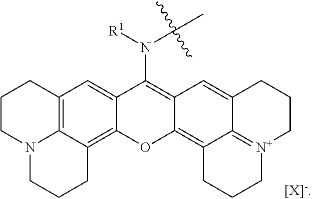

In some embodiments, the compounds of Formula (I) include:

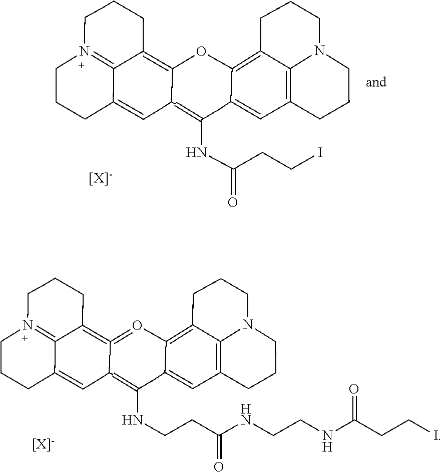

In some embodiments, the compounds of Formula (I) have the structure of Formula (IA):

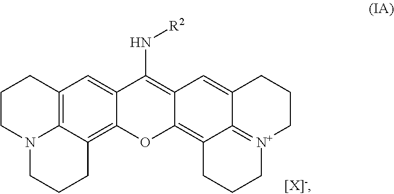

where R² and [X] are as defined above.

In some embodiments, the first carbon atom of R² is a primary carbon atom. In other embodiments, the first carbon atom of R² is a secondary carbon atom. In yet other embodiments, the first carbon atom of R² is a tertiary carbon atom. In some embodiments, R² is substituted with a group capable of participating in a “click chemistry reaction, such as BCN, DBCO, TCO, maleimide, an aldehyde, a ketone, an azide, a tetrazine, a thiol, a 1,3-nitrone, a hydrazine, or a hydroxylamine. In some embodiments, R² is substituted with an azide moiety. In some embodiments, R² is substituted with a DBCO moiety. In some embodiments, R² is substituted with a TCO moiety. In some embodiments, R² is substituted with a maleimide moiety. In some embodiments, the compounds of Formula (IA) terminate in one of a thiol-reactive group, an amine-reactive group, or a carboxy-reactive group. In some embodiments, the thiol-reactive group is selected from a haloacetyl, maleimide, iodoacetamide, aziridine, acryloyl, arylating agent, vinylsulfone, methanethiosulfonate, pyridyl disulfide, TNB-thiol and disulfide reducing agent. In some embodiments, the amine-reactive group is selected from an NHS ester (e.g., NHS, sulfo-NHS, N-hydroxy-5-norbornene-2,3-dicarboxylic acid imide), an isothiocyanate, an acyl azide, a sulfonyl chloride, a sulfodichlorophenol, pentafluorophenol, tetrafluorophenol, 4-sulfo-2,3,5,6-tetrafluorophenyl, an aldehyde, a glyoxal, an epoxide, an oxirane, a carbonate, an aryl halide, a fluorophenol ester, a sulfochlorophenol, an uranium, a carbodiimide, a phthalimide, a benzotriazole, an imidoester, an anhydride and the like. In some embodiments, the carbonyl-reactive group is hydrazine, a hydrazine derivative, or an amine. In some embodiments, R² is C₁-C₈ branched or unbranched alkyl, branched or unbranched heteroalkyl, or cycloalkyl group which is substituted with a —CO₂— maleimide. In some embodiments, R² is C₁-C₈ branched or unbranched alkyl, branched or unbranched heteroalkyl, or cycloalkyl group which is substituted with a —C₂—CO₂-maleimide. In some embodiments, R² is C₁-C₈ branched or unbranched alkyl, branched or unbranched heteroalkyl, or cycloalkyl group which is substituted with a —CO₂—NHS ester. In some embodiments, R² is C₁-C₈ branched or unbranched alkyl, branched or unbranched heteroalkyl, or cycloalkyl group which is substituted with a —C₂—CO₂—NHS ester. In some embodiments, R² is C₁-C₈ branched or unbranched alkyl, branched or unbranched heteroalkyl, or cycloalkyl group which is substituted with a group capable of participating in a “click chemistry” reaction. In some embodiments, the group capable of participating in the “click chemistry” reaction is BCN, DBCO, or TCO. In some embodiments, R² is a C₁-C₈ branched or unbranched alkyl group substituted with one or more of -Me, -Et, —CO₂⁻, —OH, -phosphoramidite, —O-phosphoramidite, -D, or a group capable of participating in a “click chemistry” reaction. In some embodiments, R² is a C₁-C₈ branched or unbranched alkyl group substituted with one or more of -Me, -Et, —CO₂⁻, —OH, -D, halogen, or a group capable of participating in a “click chemistry” reaction. In some embodiments, R² is a C₁-C₈ branched or unbranched alkyl group substituted with one or more of -Me, -Et, —CO₂⁻, —OH, halogen, or -D. In some embodiments, R² is a C₁-C₈ branched or unbranched alkyl group substituted with one or more of -Me, -Et, —CO₂⁻, —OH, -phosphoramidite, —O-phosphoramidite, -D, or a group capable of participating in a “click chemistry” reaction. In some embodiments, R² is a C₁-C₈ branched or unbranched alkyl group substituted with one or more of -Me, -Et, —CO₂⁻, —OH, -D, halogen, or a group capable of participating in a “click chemistry” reaction. In some embodiments, R² is a C₁-C₈ branched or unbranched alkyl group substituted with one or more of -Me, -Et, —CO₂, —OH, halogen, or -D. In some embodiments, R² is a C₁-C₄ branched or unbranched alkyl group substituted with one or more of -Me, -Et, —CO₂⁻, —OH, -phosphoramidite, —O-phosphoramidite, -D, a halogen, or a group capable of participating in a “click chemistry” reaction. In some embodiments, R² is a C₁-C₄ branched or unbranched alkyl group substituted with one or more of -Me, -Et, —CO₂⁻, —OH, -D, a halogen, or a group capable of participating in a “click chemistry” reaction. In some embodiments, R² is a C₁-C₄ branched or unbranched alkyl group substituted with one or more of -Me, -Et, —CO₂⁻, —OH, a halogen, or -D. Examples of compounds of Formula (IA) include, but are not limited those set forth in Tables 1a, 1b, and 1c.

TABLE 1a
Compounds of Formula (IA) where R² is a primary carbon.

| Compound | R² group |
|---|---|
| 1a | 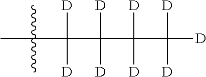 [SMILES: *C([2H])([2H])C([2H])([2H])C([2H])([2H])C([2H])([2H])[2H]] |
| 1b | 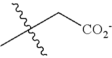 |
| 1c | 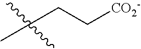 |
| 1d | 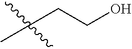 [SMILES: *CCO] |
| 1e | 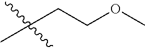 [SMILES: *CCOC] |
| 1f | 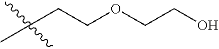 [SMILES: *CCOCCO] |
| 1g | 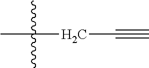 [SMILES: *CC#C] |

TABLE 1b
Compounds of Formula (IA) where R² is a secondary carbon.

| Compound | R² group |
|---|---|
| 1h | 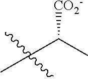 |
| 1i | 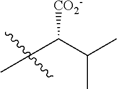 |
| 1j | 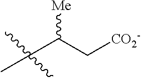 |
| 1k | 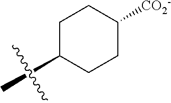 |

TABLE 1c
Compounds of Formula (IA) where R² is a tertiary carbon.

| Compound | R² group |
|---|---|
| 1l | 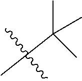 [SMILES: *C(C)(C)C] |
| 1m | 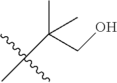 [SMILES: *C(C)(C)CO] |
| 1n | 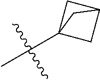 [SMILES: *C12CC(C1)C2] |
| 1o | 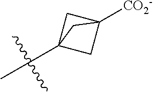 |
| 1p | 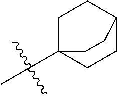 [SMILES: *C12CCC(CC1)CC2] |
| 1q | 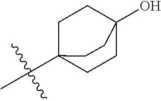 [SMILES: *C12CCC(O)(CC1)CC2] |
| 1r | 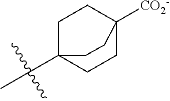 |
| 1s | 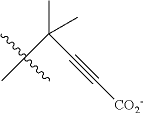 |

In some embodiments, the compounds of Formula (I) have the structure of Formula (IB):

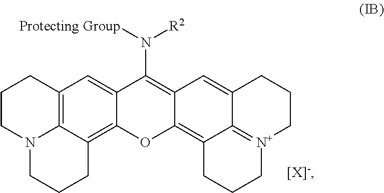

wherein
“Protecting group” is 9-fluorenylmethyl carbamate, t-Butyl carbamate, benzyl carbamate, acetamide, trifluoroacetamide, benzylamine, triphenylmethylamine, monomethoxytrityl (MMT), DMS, and p-toluenesulfonamide; and
R² and [X] are as defined herein.

In some embodiments, the first carbon atom of R² is a primary carbon atom. In some embodiments, the first carbon atom of R² is a secondary carbon atom. In some embodiments, the first carbon atom of R² is a tertiary carbon atom. In some embodiments, R² is C₁-C₈ branched or unbranched alkyl, branched or unbranched heteroalkyl, or cycloalkyl group which is substituted with a —CO₂—NHS ester. In some embodiments, R² is C₁-C₈ branched or unbranched alkyl, branched or unbranched heteroalkyl, or cycloalkyl group which is substituted with a —C₂—CO₂—NHS ester. In some embodiments, R² is C₁-C₈ branched or unbranched alkyl, branched or unbranched heteroalkyl, or cycloalkyl group which is substituted with a —CO₂-maleimide. In some embodiments, R² is C₁-C₈ branched or unbranched alkyl, branched or unbranched heteroalkyl, or cycloalkyl group which is substituted with a —C₂—CO₂-maleimide. In some embodiments, R² is C₁-C₈ branched or unbranched alkyl, branched or unbranched heteroalkyl, or cycloalkyl group which is substituted with a group capable of participating in a “click chemistry” reaction. In some embodiments, the group capable of participating in the “click chemistry” reaction is BCN, DBCO, or TCO. In some embodiments, R² is a C₁-C₈ branched or unbranched alkyl group substituted with one or more of -Me, -Et, —CO₂⁻, —OH, -phosphoramidite, —O-phosphoramidite, -D, or a group capable of participating in a “click chemistry” reaction. In some embodiments, R² is a C₁-C₈ branched or unbranched alkyl group substituted with one or more of -Me, -Et, —CO₂⁻, —OH, -D, a halogen, or a group capable of participating in a “click chemistry” reaction. In some embodiments, R² is a C₁-C₈ branched or unbranched alkyl group substituted with one or more of -Me, -Et, —CO₂⁻, —OH, a halogen, or -D. In some embodiments, R² is a C₁-C₈ branched or unbranched alkyl group substituted with one or more of -Me, -Et, —CO₂⁻, —OH, -phosphoramidite, —O-phosphoramidite, -D, or a group capable of participating in a “click chemistry” reaction. In some embodiments, R² is a C₁-C₈ branched or unbranched alkyl group substituted with one or more of -Me, -Et, —CO₂⁻, —OH, -D, a halogen, or a group capable of participating in a “click chemistry” reaction. In some embodiments, R² is a C₁-C₈ branched or unbranched alkyl group substituted with one or more of -Me, -Et, —CO₂, —OH, a halogen, or -D. In some embodiments, R² is a C₁-C₄ branched or unbranched alkyl group substituted with one or more of -Me, -Et, —CO₂⁻, —OH, -phosphoramidite, —O-phosphoramidite, -D, a halogen, or a group capable of participating in a “click chemistry” reaction. In some embodiments, R² is a C₁-C₄ branched or unbranched alkyl group substituted with one or more of -Me, -Et, —CO₂⁻, —OH, -D, a halogen, or a group capable of participating in a “click chemistry” reaction. In some embodiments, R² is a C₁-C₄ branched or unbranched alkyl group substituted with one or more of -Me, -Et, —CO₂⁻, —OH, a halogen, or -D. In some embodiments, R² is -phosphoramidite or —O-phosphoramidite.

Non-limiting examples of compounds of Formula (IB) are set forth in Table 2a, 2b, and 2c:

TABLE 2a
Compounds of Formula (IA) where R² is a primary carbon.

| Compound | R² group |
|---|---|
| 2a | 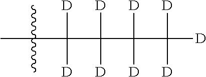 [SMILES: *C([2H])([2H])C([2H])([2H])C([2H])([2H])C([2H])([2H])[2H]] |
| 2b | 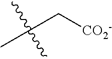 |
| 2c | 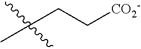 |
| 2d | 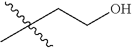 [SMILES: *CCO] |
| 2e | 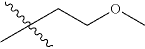 [SMILES: *CCOC] |
| 2f | 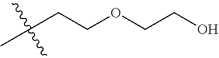 [SMILES: *CCOCCO] |
| 2g | 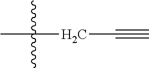 [SMILES: *CC#C] |

TABLE 2b
Compounds of Formula (IA) where R² is a secondary carbon.

| Compound | R² group |
|---|---|
| 2h | 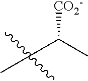 |
| 2i | 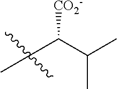 |
| 2j | 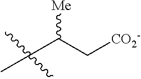 |
| 2k | 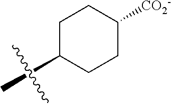 |

TABLE 2c
Compounds of Formula (IA) where R² is a tertiary carbon.

| | |
|---|---|
| 2l | 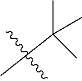 [SMILES: *C(C)(C)C] |
| 2m | 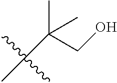 [SMILES: *C(C)(C)CO] |
| 2n | 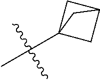 [SMILES: *C12CC(C1)C2] |
| 2o | 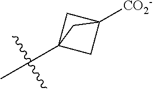 |
| 2p | 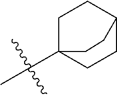 [SMILES: *C12CCC(CC1)CC2] |
| 2q | 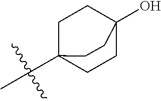 [SMILES: *C12CCC(O)(CC1)CC2] |
| 2r | 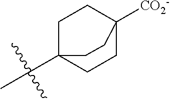 |
| 2s | 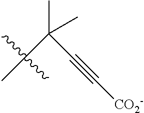 |

In some embodiments, R¹ is trifluoroacetamide and R² is a C₁-C₈ branched or unbranched alkyl group substituted with one or more of -Me, -Et, —CO₂⁻, —OH, -phosphoramidite, —O-phosphoramidite, -D, a halogen, or a group capable of participating in a “click chemistry” reaction.

In other embodiments, R¹ is trifluoroacetamide and R² is a C₁-C₈ branched or unbranched alkyl group substituted with one or more of -Me, -Et, —CO₂⁻, —OH, -D, a halogen, or a group capable of participating in a “click chemistry” reaction. In yet other embodiments, R¹ is trifluoroacetamide and R² is a C₁-C₈ branched or unbranched alkyl group substituted with one or more of -Me, -Et, —CO₂⁻, —OH, a halogen, or -D In some embodiments, R¹ is trifluoroacetamide and R² is -phosphoramidite or —O-phosphoramidite. In some embodiments, R¹ is trifluoroacetamide and R² is selected from a group consisting of:

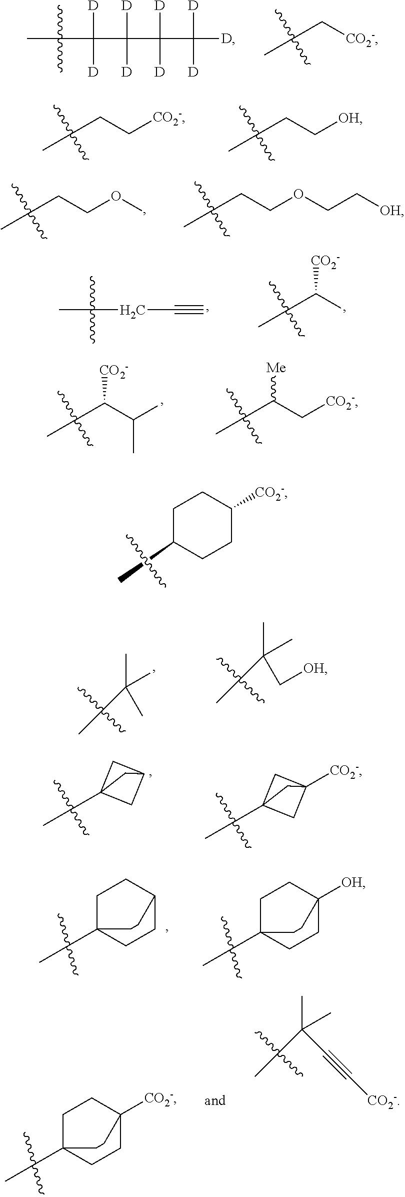

In some embodiments, the compounds of any one of Formulas (I), (IA), and (IB) have a Stokes shift of at least about 50 nm. In some embodiments, the compounds of any one of Formulas (I), (IA), and (IB) have a Stokes shift of at least about 55 nm. In some embodiments, the compounds of any one of Formulas (I), (IA), and (IB) have a Stokes shift of at least about 60 nm. In some embodiments, the compounds of any one of Formulas (I), (IA), and (IB) have a Stokes shift of at least about 65 nm. In some embodiments, the compounds of any one of Formulas (I), (IA), and (IB) have a Stokes shift of at least about 70 nm. In some embodiments, the compounds of any one of Formulas (I), (IA), and (IB) have a Stokes shift of at least about 75 nm. In some embodiments, the compounds of any one of Formulas (I), (IA), and (IB) have a Stokes shift of at least about 80 nm. In some embodiments, the compounds of any one of Formulas (I), (IA), and (IB) have a Stokes shift of at least about 85 nm. In some embodiments, the compounds of any one of Formulas (I), (IA), and (IB) have a Stokes shift of at least about 90 nm. In some embodiments, the compounds of any one of Formulas (I), (IA), and (IB) have a Stokes shift of at least about 95 nm. In some embodiments, the compounds of any one of Formulas (I), (IA), and (IB) have a Stokes shift of at least about 100 nm. In some embodiments, the compounds of any one of Formulas (I), (IA), and (IB) have a Stokes shift of at least about 105 nm. In some embodiments, the compounds of Formula (I) have a Stokes shift of at least about 110 nm.

TABLE 3
Properties of various conjugates having Formula (IA).

| Entry | Dye | | Absorption | Fluorescence | Stokes | Fluorescence/ |
|---|---|---|---|---|---|---|
| No. | No. | Amino-linker reagent | λ_{max.} [nm] | λ_{max.} [nm] | shift [nm] | absorption |
| 1 | R | Butylamine | 463 | 545 | 82 | 297 |
| 2 | 1a | d₉-Butylamine | 463 | 545 | 82 | 316 |
| 8 | 1b | Glycine | 460 | 540 | 80 | n.d. |
| 3 | 1c | β-Alanine | 459 | 550 | 91 | 267 |
| 4 | 1d | Ethanolamine | 465 | 550 | 85 | 297 |
| 5 | 1e | 2-Methoxyethylamine | 468 | 550 | 82 | 302 |
| 6 | 1f | 2-(2-Aminoethoxy)ethanol | 467 | 550 | 83 | 285 |
| 7 | 1g | Propargylamine | 474 | 555 | 81 | 246 |
| 9 | 1h | L-Alanine | 463 | 545 | 82 | 296 |
| 10 | 1i | L-Valine | 465 | 540 | 75 | 329 |
| 11 | 1j | (+/−)-3-Aminobutyric acid | 458 | 550 | 92 | 307 |
| 12 | 1k | trans-4-Aminocyclohexanecarboxylic acid | 465 | 550 | 85 | 325 |
| 13 | 1l | tert.-Butylamine | 486 | 570 | 84 | 172 |
| 14 | 1m | 2-Amino-2-methyl-1-propanol | 486 | 565 | 79 | 178 |
| 15 | 1n | 1-Bicyclo[1.1.1]pentylamine HCl | 486 | 560 | 74 | 247 |
| 16 | 1o | 3-Aminobicyclo[1.1.1]pentane-1-carboxylic acid HCl | 486 | 565 | 79 | 222 |
| 17 | 1p | Bicyclo[2.2.2]octan-1-amine HCl | 486 | 565 | 79 | 186 |
| 18 | 1q | 4-Aminobicyclo[2.2.2]octan-1-ol | 497 | 567 | 70 | 186 |
| 19 | 1r | 4-Aminobicyclo[2.2.2]octane-1-carboxylic acid | 498 | 565 | 67 | 168 |
| 20 | 1s | 4-amino-4-methylpent-2-ynoic acid | 564 | 575 | 11 | n.d. |

Table 3 above summarizes the spectroscopic properties, including the Stokes shift, of several compounds having Formula (IA). The column “amino-linker reagent” corresponds to the primary amine that reacts with R800 dye to obtain compounds R, and 1a-1s of the present disclosure. The absorption and emission maxima, and the resulting Stokes shift are indicated in nanometers.

The UPLC peak area of the fluorescence peak was divided by the peak area of the absorption peak to obtain a brightness estimate of the dyes. Compound 1b was unstable and compound is did not show properties for a LSS dye (11 nm Stokes shift), therefore brightness data was not determined (n.d.).

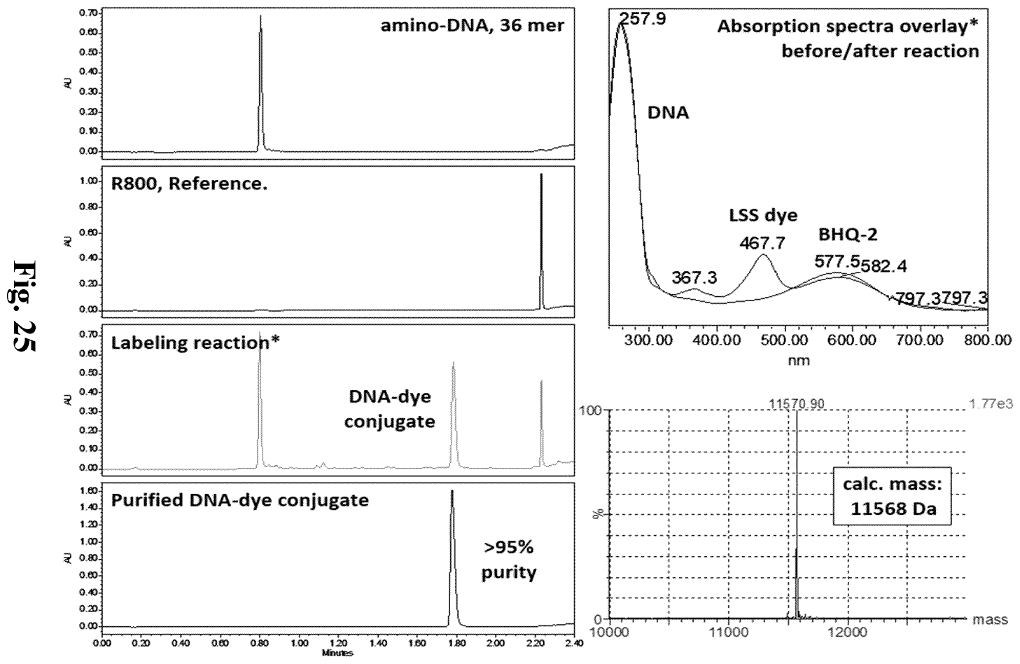

In some embodiments, the dyes having any one of Formulas (I), (IA), and (IB) are thermostable. For instance, Applicant has surprisingly discovered that the dyes of Formulas (I), (IA), and (IB) show thermostable fluorescence over a temperature range of about 25° C. to about 100° C. This is illustrated in FIG. 24, which shows the fluorescence as a function of temperature for compounds R, 1i, 1k, 1j, and 1n. No significant decrease of fluorescence up to 100° C. was observed. The somewhat more pronounced drift to higher fluorescence for compounds R and in can be explained by their increased solubility at higher temperature, since they are more hydrophobic compared to compounds 1i, 1k, 1j, which are carboxylic acids. In some embodiments, the dyes having any one of Formulas (IA) are thermostable over a temperature range of about 25° C. to about 100° C. In some embodiments, Compounds 1a-1s, as described herein, are thermostable over a temperature range of about 25° C. to about 100° C.

### Conjugates

The present disclosure also provides conjugates comprising or derived from one or more of the compounds of Formulas (I), (IA), and (IB) and a specific binding entity. In some embodiments, the one or more compounds having Formulas (I), (IA), and (IB) are directly coupled to the specific binding entity. In some embodiments, the one or more compounds having Formulas (I), (IA), and (IB) are indirectly coupled to the specific binding entity. In some embodiments, the indirect coupling is through one or more linkers.

In some embodiments, the conjugates comprise a compound derived from any one of Formulas (I), (IA), and (IB) coupled directly or indirectly to a specific binding entity. In some embodiments, the “Specific Binding Entity” is an oligonucleotide, an antibody, an antibody fragment, biotin, or streptavidin. In some embodiments, the antibody is a primary antibody. In some embodiments, the antibody is a secondary antibody.

In some embodiments, the oligonucleotide is single stranded. In some embodiments, the oligonucleotide comprises between about 5 and about 60 mer. In some embodiments, the oligonucleotide is single stranded. In some embodiments, the oligonucleotide comprises between about 5 and about 55 mer. In some embodiments, the oligonucleotide is single stranded. In some embodiments, the oligonucleotide comprises between about 5 and about 50 mer. In some embodiments, the oligonucleotide is single stranded. In some embodiments, the oligonucleotide comprises between about 5 and about 45 mer. In some embodiments, the oligonucleotide is single stranded. In some embodiments, the oligonucleotide comprises between about 5 and about 40 mer. In some embodiments, the oligonucleotide is single stranded. In some embodiments, the oligonucleotide comprises between about 5 and about 35 mer. In some embodiments, the oligonucleotide comprises between about 5 and about 30 mer. In some embodiments, the oligonucleotide comprises between about 5 and about 25 mer. In some embodiments, the oligonucleotide comprises between about 5 and about 20 mer. In some embodiments, the oligonucleotide comprises between about 5 and about 15 mer.

In some embodiments, the “Specific Binding Entity” is an oligonucleotide, and the dye having Formula (I) is coupled directly or indirectly to a 5′ end of the oligonucleotide. In some embodiments, the “Specific Binding Entity” is an oligonucleotide, the dye having Formula (I) is coupled directly or indirectly to a 3′ end of the oligonucleotide.

In some embodiments, the conjugates comprising a compound derives from any one of Formulas (I), (IA), and (IB) and a specific binding entity have the structure of Formula (II):

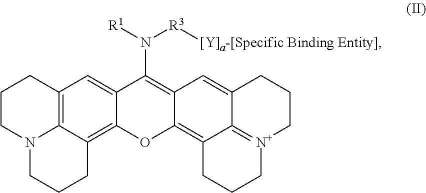

herein
R¹ is H or a protecting group;
R³ is a C₁-C₈ alkyl, heteroalkyl, or cycloalkyl group which is substituted with one or more of -Me, -Et, —CO₂—, —C₂—CO₂—, -D, or halogen;
[Specific Binding Entity] is a protein or oligonucleotide;
Y is a branched or unbranched, substituted or unsubstituted, saturated or unsaturated aliphatic or aromatic group having between 2 and about 40 carbon atoms, and optionally having one or more heteroatoms selected from O, N, or S; and
a is 0, 1, or 2.

In embodiments where the Specific Binding Entity is an oligonucleotide, the dye portion of the conjugate may be coupled to either a 5′ end or a 3′ end of the oligonucleotide. In some embodiments, the oligonucleotide, whether bound to the dye portion at a 5′ end or a 3′ end, comprises between about 5 mer and about 40 mer. In some embodiments, R³ is a C₁-C₆ alkyl, heteroalkyl, or cycloalkyl group which is substituted with one or more of -Me, -Et, —CO₂—, —C₂—CO₂—, -D, or halogen. In some embodiments, R³ is a C₁-C₄ alkyl, heteroalkyl, or cycloalkyl group which is substituted with one or more of -Me, -Et, —CO₂—, —C₂—CO₂—, -D, or halogen.

In some embodiments, Y may comprise carbonyl, amine, ester, ether, amide, imine, thione, or thiol groups. In some embodiments, Y is a branched or unbranched, linear, or cyclic, substituted or unsubstituted, saturated or unsaturated, group having between 2 and about 30 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 25 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 20 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 15 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 10 carbon atoms, and optionally having one or more heteroatoms selected from O, N, or S. In some embodiments, Y has the structure of Formula (IIIA):

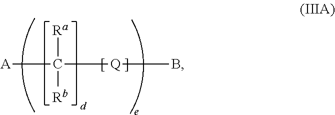

wherein d and e are integers each independently ranging from 2 to 20; Q is a bond, O, S, or N(R^{c})(R^{d}); R^{a} and R^{b} are independently H, a C₁-C₄ alkyl group, F, Cl, or N(R^{c})(R^{d}); R^{c} and R^{d} are independently CH₃ or H; and A and B are independently a branched or unbranched, linear or cyclic, substituted or unsubstituted, saturated or unsaturated group having between 1 and 12 carbon atoms and optionally having one or more O, N, or S heteroatoms.

In some embodiments, d and e are integers ranging from 2 to 6. In some embodiments, d and e are integers ranging from 2-10. In other embodiments, d and e are integers ranging from 2-5. In some embodiments, d and e are both 1. In some embodiments, A and B are independently a branched or unbranched, linear or cyclic, substituted or unsubstituted, saturated or unsaturated group having between 1 and 6 carbon atoms and optionally having one or more O, N, or S heteroatoms. are independently a branched or unbranched, linear or cyclic, substituted or unsubstituted, saturated or unsaturated group having between 1 and 4 carbon atoms and optionally having one or more O, N, or S heteroatoms. In some embodiments, Y has the structure of Formula (IIIB):

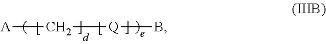

wherein
d and e are integers each independently ranging from 2 to 20;
Q is a bond, O, S, or N(R^{c})(R^{d});
R^{c} and R^{d} are independently CH₃ or H; and
A and B are independently a branched or unbranched, linear or cyclic, substituted or unsubstituted, saturated or unsaturated group having between 1 and 12 carbon atoms and optionally having one or more O, N, or S heteroatoms.

In some embodiments, Y has the structure of Formula (IIIC):

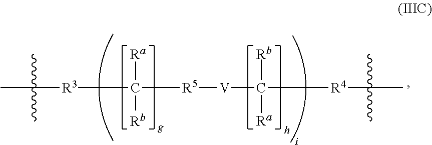

wherein each of R³ and R⁴ are independently a bond or a group selected from carbonyl, amide, imide, ester, ether, —NH, —N—, thione, or thiol;
R⁵ is a bond, a C₁-C₁₂ alkyl or heteroalkyl group including, and wherein R⁵ may include a carbonyl, an imine, or a thione;
R^{a} and R^{b} are independently H or methyl;
g and h are independently 0 or an integer ranging from 1 to 4;
i is 0, 1 or 2.

In some embodiments, R^{a} and R^{b} are each H. In some embodiments, Y is derived from:
5′-Amino-Modifier C6-TFA (GLEN RESEARCH CATALOG NO. 10-1916)
Amino-Modifier C6 dT (GLEN RESEARCH CATALOG NO. 10-1039)
Amino-L-threoninol amidite

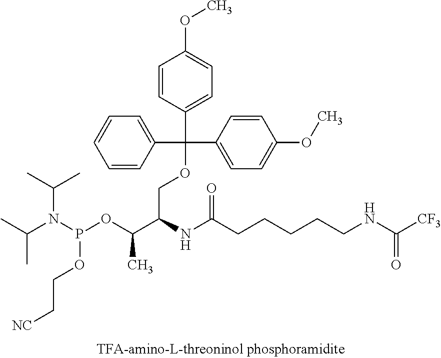 [SMILES: COc1ccc(C(OC[C@@H](NC(=O)CCCCCNC(=O)C(F)(F)F)[C@@H](C)OP(OCCC#N)N(C(C)C)C(C)C)(c2ccccc2)c2ccc(OC)cc2)cc1]

Amino-Modifier C6 dC (10-1019)
Amino-Modifier C2 dT (Glen Research Catalog No. 10-1037)
Amino-Modifier C6 dA (Glen Research Catalog No. 10-1089)
N2-Amino-Modifier C6 dG (Glen Research Catalog No. 10-1529)
Fmoc Amino-Modifier C6 dT (Glen Research Catalog No. 10-1536)
5′-Amino-Modifier 5 (Glen Research Catalog No. 10-1905)
5′-Amino-Modifier C6 (Glen Research Catalog No. 10-1906)
5′-DMS(O)MT-Amino-Modifier C6 (Glen Research Catalog No. 10-1907)
5′-Amino-Modifier C12 (Glen Research Catalog No. 10-1912)
5′-Amino-Modifier TEG CE-Phosphoramidite (Glen Research Catalog No. 10-1917)
5′-Amino-Modifier C3-TFA (Glen Research Catalog No. 10-1923)
5′-Amino-Modifier C6-PDA (Glen Research Catalog No. 10-1947)
5′-Amino-Modifier C12-PDA (Glen Research Catalog No. 10-1948)
5′-Amino-Modifier TEG PDA (Glen Research Catalog No. 10-1949)
Amino-Modifier Serinol Phosphoramidite (Glen Research Catalog No. 10-1997)
PC Amino-Modifier Phosphoramidite (Glen Research Catalog No. 10-4906)
3′-Amino-Modifier C6 dC CPG (Glen Research Catalog No. 20-2019)
3′-Amino-Modifier C6 dT CPG (Glen Research Catalog No. 20-2039)
3′-PT-Amino-Modifier C3 CPG (Glen Research Catalog No. 20-2954)
3′-PT-Amino-Modifier C6 CPG (Glen Research Catalog No. 20-2956)
3′-Amino-Modifier C7 CPG 1000 (Glen Research Catalog No. 20-2958)
3′-Amino-Modifier Serinol CPG (Glen Research Catalog No. 20-2997)
3′-PT-Amino-Modifier C6 PS (Glen Research Catalog No. 26-2956)
5′-DBCO-TEG phosphoramidite (Glen Research Catalog No. 10-1941)
DBCO-Serinol phosphoramidite (Glen Research Catalog No. 10-1998)
DBCO-dT-CE phosphoramidite (Glen Research Catalog No. 10-1539)
5′-Bromohexyl Phosphoramidite (Glen Research Catalog No. 10-1946)

In some embodiments, R¹ is H; the Specific Binding Entity is an oligonucleotide; and R³ is a C₁-C₈ alkyl, heteroalkyl, or cycloalkyl group which is substituted with one or more of -Me, -Et, -D, —C₂—CO₂—, —CO₂—, and —OH—. In some embodiments, R¹ is H; the Specific Binding Entity is an oligonucleotide; and R³ is a C₁-C₆ alkyl, heteroalkyl, or cycloalkyl group which is substituted with one or more of -Me, -Et, -D, —C₂—CO₂—, —CO₂—, and —OH—. In some embodiments, R¹ is H; the Specific Binding Entity is an oligonucleotide; and R³ is a C₁-C₄ alkyl, heteroalkyl, or cycloalkyl group which is substituted with one or more of -Me, -Et, -D, —C₂—CO₂—, —CO₂—, and —OH—. In some embodiments, R¹ is H; the Specific Binding Entity is an oligonucleotide; and R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, substituted or unsubstituted, saturated or unsaturated aliphatic or aromatic group having between 2 and about 30 carbon atoms, and optionally having one or more heteroatoms selected from O, N, or S In some embodiments, R¹ is H; the Specific Binding Entity is an oligonucleotide; and R³ is a C₁-C₆ alkyl, heteroalkyl, or cycloalkyl group which is substituted with one or more of -Me, -Et, -D, —C₂—CO₂—, —CO₂—, and —OH—; and Y is a branched or unbranched, substituted or unsubstituted, saturated or unsaturated aliphatic or aromatic group having between 2 and about 30 carbon atoms, and optionally having one or more heteroatoms selected from O, N, or S. In some embodiments, R¹ is H; the Specific Binding Entity is an oligonucleotide; and R³ is a C₁-C₄ alkyl, heteroalkyl, or cycloalkyl group which is substituted with one or more of -Me, -Et, -D, —C₂—CO₂—, —CO₂—, and —OH—, and Y is a branched or unbranched, substituted or unsubstituted, saturated or unsaturated aliphatic or aromatic group having between 2 and about 30 carbon atoms, and optionally having one or more heteroatoms selected from O, N, or S.

In some embodiments, the conjugate of Formula (II) has the structure of any one of Formulas (IIA) or (IIB):

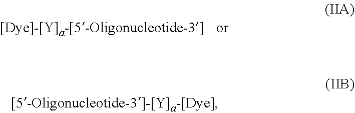

wherein
Dye is derived from any one of Formulas (I), (IA), or (IB);
Y is a branched or unbranched, substituted or unsubstituted, saturated or unsaturated aliphatic or aromatic group having between 2 and about 40 carbon atoms, and optionally having one or more heteroatoms selected from O, N, or S; and
a is 0, 1, or 2; and
Oligonucleotide is an oligonucleotide having between about 5 and about 60 mer.

In some embodiments, the Dye is derived from any one of Compounds 1a-1s (see Tables 1a, 1c, and 1c, herein). In some embodiments, Y is a branched or unbranched, linear, or cyclic, substituted or unsubstituted, saturated or unsaturated, group having between 2 and about 30 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 20 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 10 carbon atoms, and optionally having one or more heteroatoms selected from O, N, or S. In some embodiments, Y has the structure of any one of Formulas (IIIA), (IIIB), and (IIIC), as set forth herein. In some embodiments, the oligonucleotide comprises between about 5 mer and about 60 mer. In some embodiments, the oligonucleotide is single stranded. In some embodiments, the oligonucleotide comprises between about 5 and about 55 mer. In some embodiments, the oligonucleotide is single stranded. In some embodiments, the oligonucleotide comprises between about 5 and about 50 mer. In some embodiments, the oligonucleotide is single stranded. In some embodiments, the oligonucleotide comprises between about 5 and about 45 mer. In some embodiments, the oligonucleotide is single stranded. In some embodiments, the oligonucleotide comprises between about 5 and about 40 mer. In some embodiments, the oligonucleotide is single stranded. In some embodiments, the oligonucleotide comprises between about 5 and about 35 mer. In some embodiments, the oligonucleotide comprises between about 5 and about 30 mer. In some embodiments, the oligonucleotide comprises between about 5 and about 25 mer. In some embodiments, the oligonucleotide comprises between about 5 and about 20 mer. In some embodiments, the oligonucleotide comprises between about 5 and about 15 mer.

In some embodiments, the conjugate of Formula (II) has the structure of any one of Formulas (IIC) or (IID).

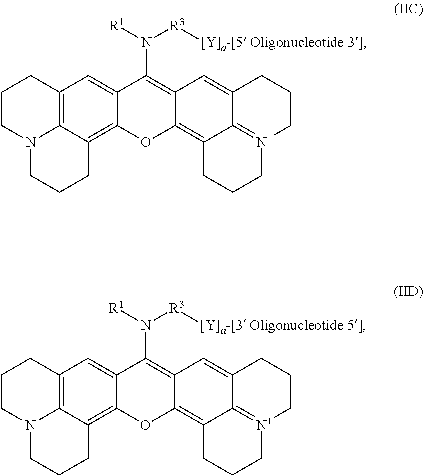

wherein
R¹ is H or a protecting group;
R³ is a C₁-C₈ alkyl, heteroalkyl, or cycloalkyl group which is substituted with one or more of -Me, -Et, —CO₂—, —C₂—CO₂—, -D, or a halogen;
Oligonucleotide is an oligonucleotide having between about 5 and about 60 mer;
Y is a branched or unbranched, substituted or unsubstituted, saturated or unsaturated aliphatic or aromatic group having between 2 and about 40 carbon atoms, and optionally having one or more heteroatoms selected from O, N, or S; and a is 0, 1, or 2.

In some embodiments, a first carbon atom of R³ is a primary carbon atom. In some embodiments, a first carbon atom of R³ is a secondary carbon atom. In some embodiments, a first carbon atom of R³ is a tertiary carbon atom. In some embodiments, R³ is a C₁-C₆ alkyl, heteroalkyl, or cycloalkyl group which is substituted with one or more of -Me, -Et, —CO₂—, —C₂—CO₂—, -D, or halogen. In some embodiments, R³ is a C₁-C₄ alkyl, heteroalkyl, or cycloalkyl group which is substituted with one or more of -Me, -Et, —CO₂—, —C₂—CO₂—, -D, or halogen. In some embodiments, Y is a branched or unbranched, linear, or cyclic, substituted or unsubstituted, saturated or unsaturated, group having between 2 and about 30 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 25 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 20 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 15 carbon atoms, and optionally having one or more heteroatoms selected from O, N, or S. In some embodiments, Y is a branched or unbranched, linear, or cyclic, substituted or unsubstituted, saturated or unsaturated, group having between 2 and about 10 carbon atoms, and optionally having one or more heteroatoms selected from O, N, or S. In some embodiments, Y may comprise carbonyl, amine, ester, ether, amide, imine, thione, or thiol groups. In some embodiments, Y has the structure of Formula (IIIA):

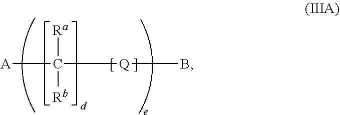

wherein d and e are integers each independently ranging from 2 to 20; Q is a bond, O, S, or N(R^{c})(R^{d}); R^{a} and R^{b} are independently H, a C₁-C₄ alkyl group, F, Cl, or N(R^{c})(R^{d}); R^{c} and R^{d} are independently CH₃ or H; and A and B are independently a branched or unbranched, linear or cyclic, substituted or unsubstituted, saturated or unsaturated group having between 1 and 12 carbon atoms and optionally having one or more O, N, or S heteroatoms. In some embodiments, d and e are integers ranging from 2 to 6.

In some embodiments, d and e are integers ranging from 2-10. In other embodiments, d and e are integers ranging from 2-5. In some embodiments, d and e are both 1. In some embodiments, A and B are independently a branched or unbranched, linear or cyclic, substituted or unsubstituted, saturated or unsaturated group having between 1 and 6 carbon atoms and optionally having one or more O, N, or S heteroatoms. are independently a branched or unbranched, linear or cyclic, substituted or unsubstituted, saturated or unsaturated group having between 1 and 4 carbon atoms and optionally having one or more O, N, or S heteroatoms. In some embodiments, Y has the structure of Formula (IIIB):

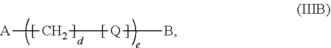

wherein
d and e are integers each independently ranging from 2 to 20;
Q is a bond, O, S, or N(R^{c})(R^{d});
R^{c} and R^{d} are independently CH₃ or H; and
A and B are independently a branched or unbranched, linear or cyclic, substituted or unsubstituted, saturated or unsaturated group having between 1 and 12 carbon atoms and optionally having one or more O, N, or S heteroatoms. In some embodiments, Y has the structure of Formula (IIIC):

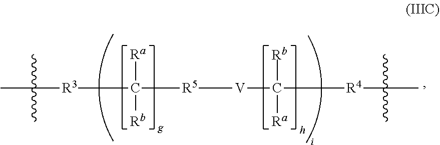

wherein each of R³ and R⁴ are independently a bond or a group selected from carbonyl, amide, imide, ester, ether, —NH, —N—, thione, or thiol;
R⁵ is a bond, a C₁-C₁₂ alkyl or heteroalkyl group including, and wherein R⁵ may include a carbonyl, an imine, or a thione;
R^{a} and R^{b} are independently H or methyl;
g and h are independently 0 or an integer ranging from 1 to 4;
i is 0, 1 or 2.

In some embodiments, R¹ is H; and R³ is a C₁-C₈ alkyl, heteroalkyl, or cycloalkyl group which is substituted with one or more of -Me, -Et, -D, —C₂—CO₂—, —CO₂—, and —OH—. In some embodiments, R¹ is H; and R³ is a C₁-C₆ alkyl, heteroalkyl, or cycloalkyl group which is substituted with one or more of -Me, -Et, -D, —C₂—CO₂—, —CO₂—, and —OH—. In some embodiments, R¹ is H; and R³ is a C₁-C₄ alkyl, heteroalkyl, or cycloalkyl group which is substituted with one or more of -Me, -Et, -D, —C₂—CO₂—, —CO₂—, and —OH—. In other embodiments, R¹ is H; R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 30 carbon atoms, and optionally having one or more heteroatoms selected from O, N, or S. In other embodiments, R¹ is H; R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 20 carbon atoms, and optionally having one or more heteroatoms selected from O, N, or S. In other embodiments, R¹ is H; R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 15 carbon atoms, and optionally having one or more heteroatoms selected from 0, N, or S. In yet other embodiments, R¹ is H; R³ is a C₁-C₈ alkyl, heteroalkyl, or cycloalkyl group which is substituted with one or more of -Me, -Et, -D, —C₂—CO₂—, —CO₂—, and —OH—; and Y is a branched or unbranched, linear, or cyclic, substituted or unsubstituted, saturated or unsaturated, group having between 2 and 10 carbon atoms, and optionally having one or more heteroatoms selected from O, N, or S. In further embodiments, R¹ is H; R³ is a C₁-C₈ alkyl, heteroalkyl, or cycloalkyl group which is substituted with one or more of -Me, -Et, -D, —C₂—CO₂—, —CO₂—, and —OH—; and Y has the structure of any one of Formulas (IIIA), (IIIB), and (IIIC). In some embodiments, the oligonucleotide comprises between about 5 mer and about 60 mer. In some embodiments, the oligonucleotide is single stranded. In some embodiments, the oligonucleotide comprises between about 5 and about 55 mer. In some embodiments, the oligonucleotide is single stranded. In some embodiments, the oligonucleotide comprises between about 5 and about 50 mer. In some embodiments, the oligonucleotide is single stranded. In some embodiments, the oligonucleotide comprises between about 5 and about 45 mer. In some embodiments, the oligonucleotide is single stranded. In some embodiments, the oligonucleotide comprises between about 5 and about 40 mer. In some embodiments, the oligonucleotide is single stranded. In some embodiments, the oligonucleotide comprises between about 5 and about 35 mer. In some embodiments, the oligonucleotide comprises between about 5 and about 30 mer. In some embodiments, the oligonucleotide comprises between about 5 and about 25 mer. In some embodiments, the oligonucleotide comprises between about 5 and about 20 mer. In some embodiments, the oligonucleotide comprises between about 5 and about 15 mer.

The present disclosure is also directed to conjugates comprising a compound of Formula (I) and a hapten or an enzyme (e.g., alkaline phosphatase; horse radish peroxidase). In some embodiments, the compounds having Formula (I) is directly coupled to the hapten or the enzyme. In some embodiments, the compounds having Formula (I) is indirectly coupled to the hapten or the enzyme. In some embodiments, the indirect coupling is through one or more linkers. In some embodiments, the hapten is a pyrazole (e.g., nitropyrazoles); a nitrophenyl compounds; a benzofurazan; a triterpene; a ureas (e.g., phenyl ureas); a thiourea (e.g., phenyl thioureas); a rotenone or a rotenone derivative; an oxazole (e.g., oxazole sulfonamides); a thiazole (e.g., thiazole sulfonamides); a coumarin or a coumarin derivatives; or a cyclolignan. In some embodiments, the happen is di-nitrophenyl, biotin, digoxigenin, and fluorescein, and any derivatives or analogs thereof. Other haptens are described in U.S. Pat. Nos. 8,846,320; 8,618,265; 7,695,929; 8,481,270; and 9,017,954, the disclosures of which are incorporated herein by reference in their entirety.

### TaqMan® Probes

The present disclosure also provides TaqMan® probes, where a first dye of the TaqMan® probe is derived from any one of the compounds of Formulas (I), (IA), and (IB), and where a second dye is a quencher. TaqMan® probes may be used to conduct a TaqMan® assay, for example, as known in the art. As used herein, the terms “TaqMan® probe” and “hydrolysis probe” may be understood interchangeably. In some embodiments, the first dye derived from a compound having Formula (I) and the quencher are located near the termini of the probe, and in some such embodiments, the compound having Formula (I) is located near the 5′ terminus and the quencher is located near the 3′ terminus. The term “3′-terminal” may be understood in the broadest sense as understood in the art. Further, the terms “3′ terminus” and “3′ end” may be understood interchangeably as known in the art. Also, it should be understood that the terms “3′ terminus” and “3′ end” as used herein may refer to the 5′ end of the nucleotide strand but may not exclude that at the 3′ end another molecular moiety (such as, e.g., a fluorophore, a quencher, a binding moiety or the like) is added to the 3′ end of the probe.

The TaqMan® probe may hybridize to its target sequence. Further, a composition including a TaqMan® probe may further comprise a pair of primers, e.g., one forward and one reverse primer. These primers are generally unlabeled. Further, generally, the forward primer binds upstream, the reverse primer downstream of the band, such that the TaqMan® probe binds to a sequence that is a part of the strand that is amplified. A PCR reaction as well-known in the art is conducted. Thus, the target DNA is melted, then conditions are chosen that enable the annealing of the primers and the probe to the target DNA. Subsequently, conditions are chosen that enable the DNA polymerase to amplify the DNA strand between the primers. In the context of the TaqMan® assay, the DNA polymerase generally has a 5′ to 3′ exonuclease activity. Also, the DNA polymerase may be Taq polymerase or a functional variant thereof. When the DNA polymerase comes to the TaqMan® probe, the 5′ end is cleaved off. Thereby, the compound having orb derived from Formula (I), or quencher bound to the 5′ terminal nucleotide(s) is also cleaved off. In some embodiments, the compound having or derived from Formula (I) is cleaved off Consequently, the compound having or derived from Formula (I) and the quencher may diffuse in different directions. The spatial distance between both may be significantly increased and the fluorescence occurred by the compound having or derived from Formula (I) is significantly increased as it is not quenched by the dark quencher any longer. Also, the TaqMan® assay may be analyzed in real-time. The TaqMan® assay may also be conducted during a life-time PCR method. It may also be conducted quantitatively in a qPCR reaction.

A TaqMan® assay using the probes of the present disclosure may be used for the discrimination of alleles, genotyping, bacterial identification assays, DNA quantification, and the determination of the viral load in clinical specimen, gene expression assays and verification of microarray results. It may also be used for the discrimination of alleles, genotyping, and bacterial identification assays. Genotyping may be single nucleotide polymorphisms (SNP) genotyping, for example, and therefore include the determination of a genotype at defined a locus of interest in a sample, wherein the locus is a single nucleotide. Alternatively, genotyping may be copy number variant (CNV) genotyping. A copy number variant (CNV) is a segment of DNA in which differences of copy-number (number of copies of a DNA sequence or portions thereof) have been found by comparison of two or more genomes. As discussed above, sequences (and loci of various SNPs and CNVs) may be obtained from databases such as The Database of Genomic Variants (DGV), the NCBI dbSNP database, the UCSC Genome Bioinformatics Site, the DatabasE of Chromosomal Imbalance and Phenotype in Humans using Ensembl Resources (DECIPHER), the HapMap Project, the Sanger Institute Copy Number Variation Project and the Human Structural Variation Project.

The present disclosure provides probes, e.g., TaqMan® probes, having the structure of Formula (IV):

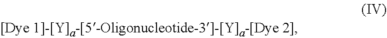

where
one of [Dye 1] or [Dye 2] is derived from any one of Formulas (I), (IA), or (IB); and another one of Dye 1 or Dye 2 is a quencher;
Oligonucleotide is an oligonucleotide having between about 5 and about 60 mer;
Y is a branched or unbranched, substituted or unsubstituted, saturated or unsaturated aliphatic or aromatic group having between 2 and about 40 carbon atoms, and optionally having one or more heteroatoms selected from O, N, or S; and a is 0, 1, or 2.

In some embodiments, the Quencher is a molecule which decreases the fluorescence intensity of Dye 1 or Dye2. In other embodiments, the Quencher is selected from Deep Dark Quencher DDQ-I, DABCYL, Eclipse® Dark quencher, Iowa Black® FQ, Iowa Black® RQ, Black Hole Quencher® series (BHQ-0, BHQ-1, BHQ-2, BHQ-3), QSY-7, DDQ-II, Iowa Black® RQ, QSY-21, Black Berry Quencher (BBQ-650, available from LGC Biosearch); IDT double quencher (ZEN Quencher; TAO Quencher); Onyx Quencher (available from Milipore Sigma), and TAMRA quencher. In some embodiments, the one of Dye 1 or Dye 2 has a Stokes shift of at least about 60 nm, of at least about 70 nm, of at least about 80 nm, of at least about 90 nm, of at least about 100 nm, etc. In some embodiments, the one of [Dye 1] or [Dye 2] is derived from a compound having Formula (IA). In other embodiments, the Dye is derived from a compound having Formula (IA) and where R² is

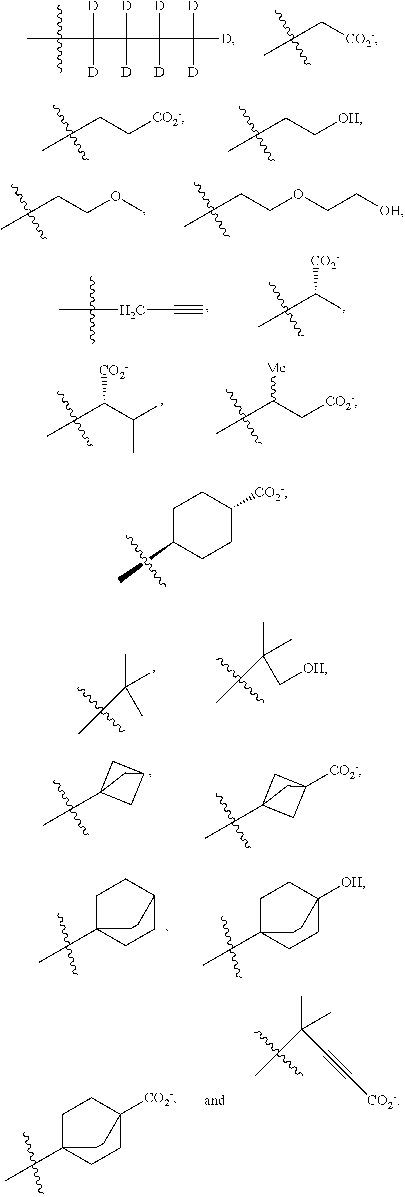

### Tags Probes

The present disclosure further provides conjugates having the structure of Formula (V):

[(Oligomer 1)(Dye)]-Linker-[(Oligomer 2)(Q1)] (V),
wherein
Oligomers 1 and 2 are each different and are oligomers having between about 5 mer and about 30 mer;
Dye is derived from any one of (I), (IA), or (IB);
Q1 is a quencher; and
Linker is a substituted or unsubstituted aliphatic, heteroaliphatic, aromatic, or heteroaromatic group having between about 5 and about 40 carbon atoms.

In some embodiments, Linker is a substituted or unsubstituted aliphatic, heteroaliphatic, aromatic, or heteroaromatic group having between about 5 and about 30 carbon atoms. In some embodiments, Linker is a substituted or unsubstituted aliphatic, heteroaliphatic, aromatic, or heteroaromatic group having between about 5 and about 25 carbon atoms. In some embodiments, Linker is a substituted or unsubstituted aliphatic, heteroaliphatic, aromatic, or heteroaromatic group having between about 5 and about 20 carbon atoms. In some embodiments, Linker is a substituted or unsubstituted aliphatic, heteroaliphatic, aromatic, or heteroaromatic group having between about 5 and about 15 carbon atoms. In some embodiments, at least one of Oligomer 1, Oligomer 2, or the Linker includes a nuclease susceptible cleavage site. In some embodiments, Oligomers 1 and 2 may comprise DNA, L-DNA, RNA, L-RNA, LNA, L-LNA, PNA (peptide nucleic acid, as described in Nielsen et al., U.S. Pat. No. 5,539,082), BNA (bridged nucleic acid, for example, 2′,4′-BNA(NC) [2′-O,4′-C-aminomethylene bridged nucleic acid] as described in Rahman et al., J. Am. Chem. Soc. 2008; 130(14):4886-96), L-BNA etc. (where the “L-XXX” refers to the L-enantiomer of the sugar unit of the nucleic acids) or any other known variations and modifications on the nucleotide bases, sugars, or phosphodiester backbones. In some embodiments, one of Oligomer 1 or Oligomer 2 includes or is comprised entirely of L-DNA. In other embodiments, Oligomer 1 includes or is comprised entirely of L-DNA. In yet other embodiments, Oligomer 1 is entirely comprised of L-DNA. In some embodiments, Linker is an unsubstituted aliphatic, heteroaliphatic, aromatic, or heteroaromatic group having between about 5 and about 30 carbon atoms. In other embodiments, Linker is an unsubstituted aliphatic, heteroaliphatic, aromatic, or heteroaromatic group having between about 5 and about 25 carbon atoms. In yet other embodiments, Linker is an unsubstituted aliphatic, heteroaliphatic, aromatic, or heteroaromatic group having between about 5 and about 25 carbon atoms. In some embodiments, the Dye has a Stokes shift of at least about 60 nm, of at least about 70 nm, of at least about 80 nm, of at least about 90 nm, of at least about 100 nm, etc. In some embodiments, the Dye is derived from a compound having Formula (IA). In other embodiments, the Dye is derived from a compound having Formula (IA) where R² is

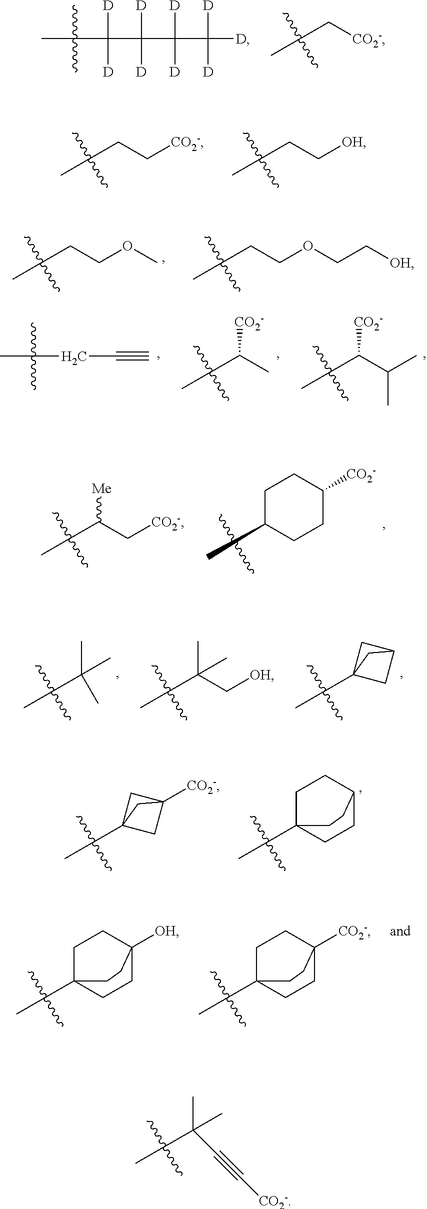

The present disclosure further provides an intermediate having the structure of Formula (VI):

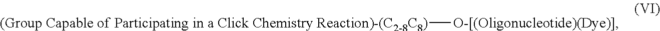

where
Oligonucleotide is an oligonucleotide having between about 5 and about 60 mer; and
Dye is derived from any one of (I), (IA), or (IB).

In some embodiments, the Dye has a Stokes shift of at least about 60 nm, of at least about 70 nm, of at least about 80 nm, of at least about 90 nm, of at least about 100 nm, etc. In some embodiments, the Dye is derived from a compound having Formula (IA). In other embodiments, the Dye is derived from a compound having Formula (IA) where R² is

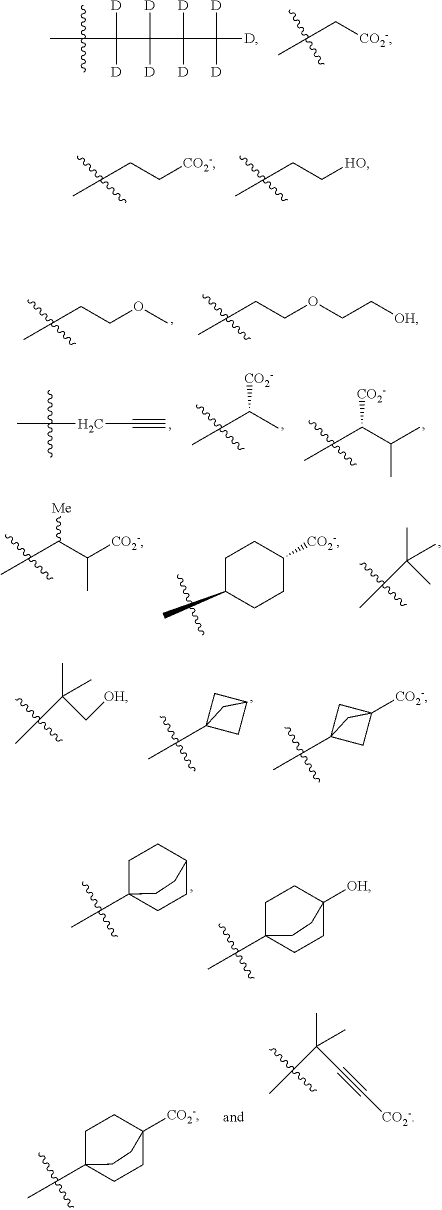

### Kits

The present disclosure also provides kits comprising at least two compounds having any one of Formulas (IA). The present disclosure also provides kits comprising at least three compounds having any one of Formulas (IA). The present disclosure also provides kits comprising at least four compounds having any one of Formulas (IA). The present disclosure also provides kits comprising at least five compounds having any one of Formulas (IA). The present disclosure also provides kits comprising at least six compounds having any one of Formulas (IA). The present disclosure also provides kits comprising seven or more compounds having any one of Formulas (IA).

In some embodiments, at least one of the compounds having Formula (IA) included within any kit have a stokes shift of greater than about 50 nm. In some embodiments, at least one of the compounds having Formula (IA) included within any kit have a shift of greater than about 60 nm. In some embodiments, at least one of the compounds having Formula (IA) included within any kit have a stokes shift of greater than about 70 nm. In some embodiments, at least one of the compounds having Formula (IA) included within any kit have a stokes shift of greater than about 80 nm. In some embodiments, at least one of the compounds having Formula (IA) included within any kit have a stokes shift of greater than about 90 nm. In some embodiments, at least one of the compounds having Formula (IA) included within any kit have a stokes shift of greater than about 100 nm. In some embodiments, at least one of the compounds having Formula (IA) included within any kit have a stokes shift of greater than about 110 nm.

### FRET Pairs

The present disclosure also provides kits comprising a FRET pair. FRET is a form of molecular energy transfer (MET), a process by which energy is passed non-radioactively between a donor molecule and an acceptor molecule. FRET arises from the properties of certain chemical compounds; when excited by exposure to particular wavelengths of light, they emit light (i.e., they fluoresce) at a different wavelength. Such compounds are termed fluorophores or fluorescent labels. In FRET, energy is passed non-radioactively over a long distance (e.g., 10-100 Angstroms) between a donor molecule, which may be a fluorophore, and an acceptor molecule, which may be a quencher or another fluorophore. The donor absorbs a photon and transfers this energy non-radioactively to the acceptor (Forster, 1949, Z. Naturforsch. A4:321-327; Clegg, 1992, Methods Enzymol. 211:353-388).

When two fluorophores whose excitation and emission spectra overlap are in close proximity, excitation of one fluorophore will cause it to emit light at wavelengths that are absorbed by, and that stimulate, the second fluorophore, causing it in turn to fluoresce. In other words, the excited-state energy of the first (donor) fluorophore is transferred by a resonance induced dipole-dipole interaction to the neighboring second (acceptor) fluorophore. As a result, the lifetime of the donor molecule is decreased and its fluorescence is quenched, while the fluorescence intensity of the acceptor molecule is enhanced and depolarized. When the excited-state energy of the donor is transferred to a non-fluorophore acceptor, the fluorescence of the donor is quenched without subsequent emission of fluorescence by the acceptor. In this case, the acceptor functions as a quencher.

Pairs of molecules that can engage in FRET are termed FRET pairs. In order for energy transfer to occur, the donor and acceptor molecules must typically be in close proximity (e.g., up to 70 to 100 Angstroms) (Clegg, 1992, Methods Enzymol. 211:353-388; Selvin, 1995, Methods Enzymol. 246:300-334). The efficiency of energy transfer falls off rapidly with increased distance between the donor and acceptor molecules. Effectively, this means that FRET can most efficiently occur up to distances of about 70 Angstroms.

In some embodiments of the present disclosure, a FRET pair comprises a first member including a dye of or derived from Formula (I) coupled directly or indirectly to a first oligonucleotide; and a second member including a second oligonucleotide coupled directly or indirectly to a quencher. In some embodiments, the first member of the FRET Pair includes a conjugate having any one of Formulas (IIA), (IIB), (IIC) or (IID).

In some embodiments, a FRET pair comprises a first member having Formula (VIIA) and a second member having Formula (VIIB):

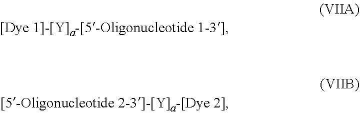

wherein
one of Dye 1 or Dye 2 are derived from any one of Formulas (I), (IA), or (IB);
another one of Dye 1 or Dye 2 is a Quencher;
each Y is independently a branched or unbranched, linear, or cyclic, substituted or unsubstituted, saturated or unsaturated, group having between 2 and about 40 carbon atoms, and optionally having one or more heteroatoms selected from O, N, or S;
a is 0, 1, or 2; and
Oligonucleotide 1 and Oligonucleotide 2 are different.

In some embodiments, the one of Dye 1 or Dye 2 has a Stokes shift of at least about 60 nm, of at least about 70 nm, of at least about 80 nm, of at least about 90 nm, of at least about 100 nm, etc. In some embodiments, one of Dye 1 or Dye 2 is derived from Formula (IA) where R² is

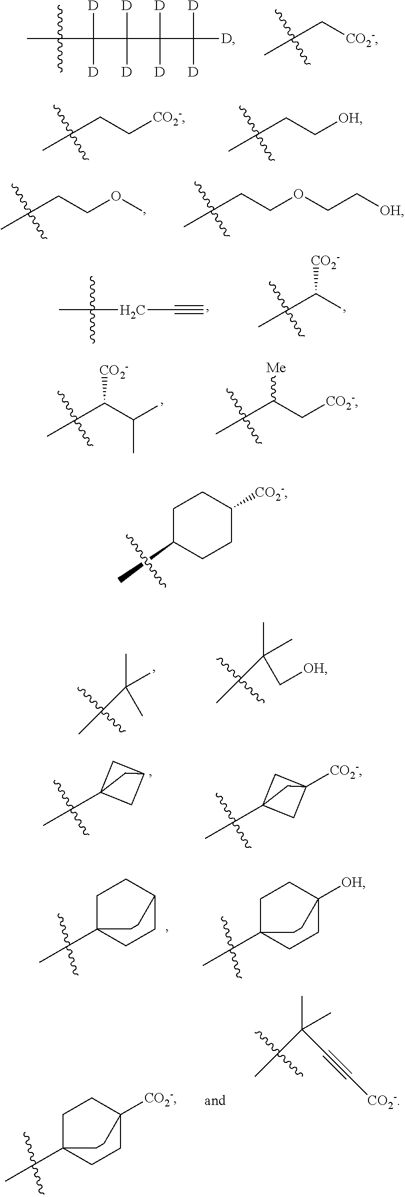

Any quencher may be used without limitation in the compositions described herein provided that it decreases the fluorescence intensity of the dye of or derived from Formula (I) that is being used. Quenchers commonly used for FRET include, but are not limited to, Deep Dark Quencher DDQ-I, DABCYL, Eclipse® Dark quencher, Iowa Black® FQ, BHQ-1, QSY-7, BHQ-2, DDQ-II, Iowa Black® RQ, QSY-21, and Black Hole Quencher® BHQ-3. Quenchers for use in the compositions provided herein may be obtained commercially, for example, from Eurogentec (Belgium), Epoch Biosciences (Bothell, Wash.), Biosearch Technologies (Novato Calif.), Integrated DNA Technologies (Coralville, Iowa) and Life Technologies (Carlsbad, Calif.).

In some embodiments, each Y is independently a branched or unbranched, linear, or cyclic, substituted or unsubstituted, saturated or unsaturated, group having between 2 and about 30 carbon atoms, and optionally having one or more heteroatoms selected from O, N, or S. In some embodiments, each Y is independently a branched or unbranched, linear, or cyclic, substituted or unsubstituted, saturated or unsaturated, group having between 2 and about 25 carbon atoms, and optionally having one or more heteroatoms selected from O, N, or S. In some embodiments, each Y is independently a branched or unbranched, linear, or cyclic, substituted or unsubstituted, saturated or unsaturated, group having between 2 and about 20 carbon atoms, and optionally having one or more heteroatoms selected from O, N, or S. In some embodiments, each Y is independently a branched or unbranched, linear, or cyclic, substituted or unsubstituted, saturated or unsaturated, group having between 2 and about 15 carbon atoms, and optionally having one or more heteroatoms selected from O, N, or S.

In some embodiments, Oligonucleotides 1 and 2 includes a nucleotide modification selected from Locked Nucleic Acid (LNA), Peptide Nucleic Acid (PNA), Bridged Nucleic Acid (BNA), 2′-O alkyl substitution, L-enantiomeric nucleotide, or combinations thereof. In some embodiments, the nucleotide modification comprises LNA.

In some embodiments, the present disclosure provides a method of determining a genotype at a locus of interest in a sample comprising genetic material, the method comprising the steps of: contacting the genetic material with a first probe having Formula (VIIA) and a second probe having Formula (VIIB); and detecting the binding of one of the first and second probe to the genetic material, thereby determining the genotype at the locus. In some embodiments, the first and second probes each have a 5′ end opposite a 3′ end and a predetermined number of nucleotides (e.g., 4, 6, 8, 10, 12, 16, 20 nucleotides) comprising at least one DNA nucleotide and a predetermined number of locked nucleic acid nucleotides (e.g., at least five 2, 3, 4, 5, 6, 7, 8 locked nucleotides). In some embodiments, the nucleotides of the first probe comprising a first discriminating position and the nucleotides of the second probe comprising a second discriminating position at a same nucleotide location in the second probe as the first discriminating position in the first probe, the first discriminating position comprising a different nucleobase than the second discriminating position, wherein the nucleobases at the other nucleotides of the first and second probes being the same.

### Tags Probe

The present disclosure also provides for kits comprising (i) a conjugate having Formula (V), and (ii) a conjugate having Formula (VIII):

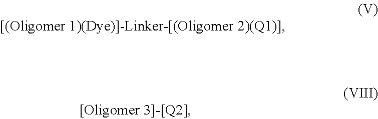

wherein
Dye is derived from any one of (I), (IA), or (IB);
Oligomers 1, 2, and 3 are each different and are oligomers having between about 5 mer and about 30 mer;
Q1 and Q2 are the same or different quenchers; and
Linker is a substituted or unsubstituted aliphatic, heteroaliphatic, aromatic, or heteroaromatic group having between about 5 and about 40 carbon atoms.

In some embodiments, Linker is a substituted or unsubstituted aliphatic, heteroaliphatic, aromatic, or heteroaromatic group having between about 5 and about 30 carbon atoms. In some embodiments, Linker is a substituted or unsubstituted aliphatic, heteroaliphatic, aromatic, or heteroaromatic group having between about 5 and about 25 carbon atoms. In some embodiments, Linker is a substituted or unsubstituted aliphatic, heteroaliphatic, aromatic, or heteroaromatic group having between about 5 and about 20 carbon atoms. In some embodiments, Linker is a substituted or unsubstituted aliphatic, heteroaliphatic, aromatic, or heteroaromatic group having between about 5 and about 15 carbon atoms. In some embodiments, one of Oligomer 1 or Oligomer 2 includes or is comprised entirely of L-DNA. In other embodiments, Oligomer 1 includes or is comprised entirely of L-DNA. In yet other embodiments, Oligomer 1 is entirely comprised of L-DNA. In other embodiments, Oligomer 2 includes or is comprised entirely of L-DNA. In yet other embodiments, Oligomer 2 is entirely comprised of L-DNA. In some embodiments, Q1 and Q2 are the same. In other embodiments, Q1 and Q2 are different. In some embodiments, the Dye has a Stokes shift of at least about 60 nm, of at least about 70 nm, of at least about 80 nm, of at least about 90 nm, of at least about 100 nm, etc. In some embodiments, the Dye in the kit is derived from Formula (IA) where R² is

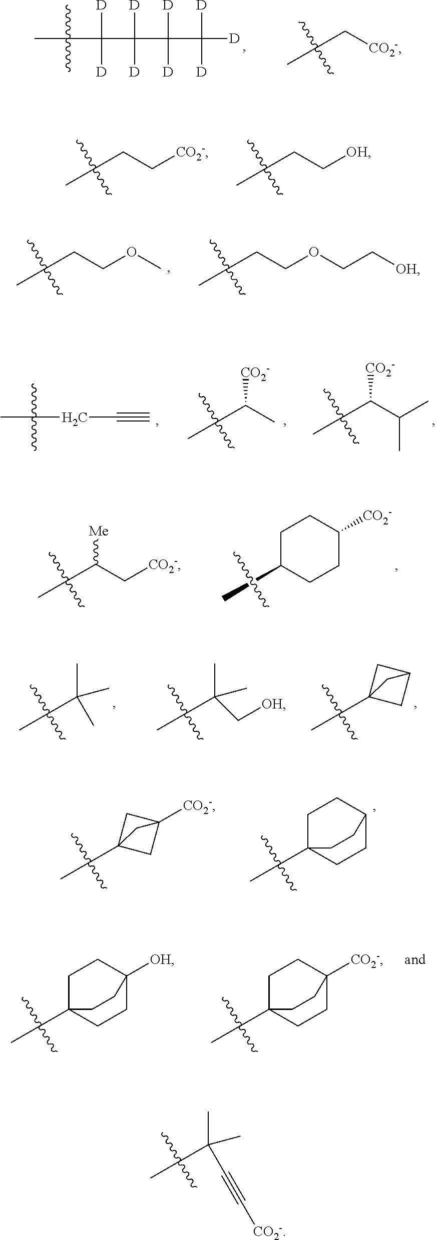

In some embodiments, the present disclosure provides a kit for detecting two or more target nucleic acid sequences in a sample comprising:
(a) two or more pairs of oligonucleotide primers with sequences that are complementary to each strand of the two or more target nucleic acid sequences;
(b) at least one oligonucleotide probe comprising two distinct portions:
(i) an annealing portion comprising a sequence at least partially complementary to one of the two or more target nucleic acid sequences and anneals within the one of the two or more target nucleic acid sequences, wherein the annealing portion comprises a first quencher moiety; and
(ii) a tag portion attached to the 5′ terminus or to the 3′ terminus of the annealing portion or attached via a linker between the 5′ terminus and the 3′ terminus of the annealing portion, and comprising a nucleotide sequence that is non-complementary to the one of the two or more target nucleic acid sequences, wherein the tag portion comprises a compound derived from Formula (IA) and whose detectable signal is capable of being quenched by the first quencher moiety on the annealing portion, wherein the compound derived from Formula (IA) is separated from the first quenching moiety by a nuclease susceptible cleavage site;
(c) at least one quenching oligonucleotide comprising a nucleotide sequence at least partially complementary to the tag portion of the oligonucleotide probe and hybridizes to the tag portion to form a duplex, wherein the quenching oligonucleotide comprises a second quencher moiety which quenches the detectable signal generated by the compound derived from Formula (IA) on the tag portion when the quenching oligonucleotide is hybridized to the tag portion.

In some embodiments, the tag portion is attached to the 5′ terminus of the annealing portion. In some embodiments, the tag portion is attached via a linker between the 5′ terminus and the 3′ terminus of the annealing portion. In some embodiments, the tag portion of the oligonucleotide probe or the quenching oligonucleotide or both the tag portion of the oligonucleotide probe and the quenching oligonucleotide contains one or more nucleotide modifications. In some embodiments, the one or more nucleotide modifications comprises a nucleotide modification selected from Locked Nucleic Acid (LNA), Peptide Nucleic Acid (PNA), Bridged Nucleic Acid (BNA), 2′-O alkyl substitution, L-enantiomeric nucleotide, or combinations thereof. In some embodiments, the nucleotide modification comprises LNA. In some embodiments, the nucleotide modification comprises PNA. In some embodiments, the nucleotide modification comprises BNA. In some embodiments, the nucleotide modification comprises L-enantiomeric nucleotide. In some embodiments, the nucleotide modification comprises L-enantiomeric LNA (L-LNA). In some embodiments, the nucleotide modification comprises 2′-O alkyl substitution. In some embodiments, the nucleotide modification comprises 2′-O methyl substitution (2′-OMe).

A method for amplification and detection of a target nucleic acid in a sample comprising the steps of.
(a) contacting the sample containing the target nucleic acid in a single reaction vessel with
(i) one pair of oligonucleotide primers, each oligonucleotide primer capable of hybridizing to opposite strands of a subsequence of the target nucleic acid;
(ii) an oligonucleotide probe that comprises an annealing portion and a tag portion, wherein the tag portion comprises a nucleotide sequence non-complementary to the target nucleic acid sequence, wherein the annealing portion comprises a nucleotide sequence at least partially complementary to the target nucleic acid sequence and hybridizes to a region of the subsequence of the target nucleic acid that is bounded by the pair of oligonucleotide primers, wherein the probe further comprises an interactive dual label comprising a compound having (or derived from) Formula (IA) located on the tag portion and a first quencher moiety located on the annealing portion and wherein the compound having (or derived from) Formula (IA) is separated from the first quencher moiety by a nuclease susceptible cleavage site; and
wherein prior to step (b), the tag portion is reversibly bound in a temperature-dependent manner to a quenching oligonucleotide comprising a nucleotide sequence at least partially complementary to the tag portion of the oligonucleotide probe and binds to the tag portion by hybridization, wherein the quenching oligonucleotide comprises at least a second quencher moiety capable of quenching the compound having (or derived from) Formula (IA) on the tag portion when the quenching oligonucleotide is bound to the tag portion;
(b) following step (a), amplifying the target nucleic acid by polymerase chain reaction (PCR) using a nucleic acid polymerase having 5′ to 3′ nuclease activity such that during an extension step of each PCR cycle, the nuclease activity of the polymerase allows cleavage and separation of the tag portion from the first quencher moiety on the annealing portion of the probe;
(c) measuring a suppressed signal from the compound having (or derived from) Formula (IA) at a first temperature at which the quenching oligonucleotide is bound to the tag portion;
(d) increasing temperature to a second temperature at which the quenching oligonucleotide is not bound to the tag portion;
(e) measuring a temperature corrected signal from the compound having (or derived from) Formula (IA) at the second temperature;
(f) obtaining a calculated signal value by subtracting the suppressed signal detected at the first temperature from the temperature corrected signal detected at the second temperature;
(g) repeating steps (b) through (f) through multiple PCR cycles;
(h) measuring the calculated signal values from the multiple PCR cycles to detect the presence of the target nucleic acid.

A method for amplification and detection of a target nucleic acid in a sample comprising the steps of:
(a) contacting the sample containing the target nucleic acid in a single reaction vessel with
(i) one pair of oligonucleotide primers, each oligonucleotide primer capable of hybridizing to opposite strands of a subsequence of the target nucleic acid;
(ii) an oligonucleotide probe that comprises an annealing portion and a tag portion, wherein the tag portion comprises a nucleotide sequence non-complementary to the target nucleic acid sequence, wherein the annealing portion comprises a nucleotide sequence at least partially complementary to the target nucleic acid sequence and hybridizes to a region of the subsequence of the target nucleic acid that is bounded by the pair of oligonucleotide primers, wherein the probe further comprises an interactive dual label comprising a compound having (or derived from) Formula (IA) located on the tag portion and a first quencher moiety located on the annealing portion and wherein the compound having (or derived from) Formula (IA) is separated from the first quencher moiety by a nuclease susceptible cleavage site; and
wherein prior to step (b), the tag portion is reversibly bound in a temperature-dependent manner to a quenching oligonucleotide comprising a nucleotide sequence at least partially complementary to the tag portion of the oligonucleotide probe and binds to the tag portion by hybridization, wherein the quenching oligonucleotide comprises at least a second quencher moiety capable of quenching the compound having (or derived from) Formula (IA) on the tag portion when the quenching oligonucleotide is bound to the tag portion;
(b) following step (a), amplifying the target nucleic acid by polymerase chain reaction (PCR) using a nucleic acid polymerase having 5′ to 3′ nuclease activity such that during an extension step of each PCR cycle, the nuclease activity of the polymerase allows cleavage and separation of the tag portion from the first quencher moiety on the annealing portion of the probe;
(c) measuring one or more signals from the compound having (or derived from) Formula (IA) at a first temperature at which the quenching oligonucleotide is bound to the tag portion;
(d) measuring one or more signals from the compound having (or derived from) Formula (IA) at a second temperature, which is higher than the first temperature, at which the quenching oligonucleotide is not bound to the tag portion;
(e) obtaining a calculated signal value by subtracting a median or average of the one or more signals detected at the first temperature from a median or average of the one or more signals detected at the second temperature;
whereby a calculated signal value that is higher than a threshold signal value allows determination of the presence of the target nucleic acid.

In some embodiments, the PCR amplification of step (b) is allowed to reach an endpoint beyond the log phase of amplification. In some embodiments, the tag portion comprises a modification such that it is not capable of being extended by the nucleic acid polymerase. In some embodiments, the tag portion of the oligonucleotide probe or the quenching oligonucleotide or both the tag portion and the quenching oligonucleotide contain one or more nucleotide modifications. In some embodiments, the one or more nucleotide modifications is selected from the group consisting of Locked Nucleic Acid (LNA), Peptide Nucleic Acid (PNA), Bridged Nucleic Acid (BNA), 2′-O alkyl substitution, L-enantiomeric nucleotide, and combinations thereof.

Other methods of using TAGS probes are set forth within U.S. Pat. Nos. 11,028,433, 11,034,997, and 11,345,958; and in United States Patent Publication No. 2021/0269857, the disclosures of which are hereby incorporated by reference herein in their entireties.

### Synthesis

The present disclosure provides methods of synthesizing the compounds of any one of Formulas (I), (IA), and (IB) and derivatives and analogs thereof. The present disclosure also provides methods of synthesizing intermediates.

### Overview

The compounds of Formulas (I) and (IB) where R¹ is a protecting group allows the formation of stable amino-protection under the conditions of solid-phase synthesis with phosphoramidite chemistry in the preparation of nucleic acids. For instance, the amino-group may be protected as trifluoroacetamide (TFA, trifluoroacetyl protection group), resulting in Compounds 2a-2s (see Tables 2a-2c), where R¹ is a TFA protecting group. In some embodiments, the TFA group may be cleaved during the deprotection conditions that are common in solid-phase synthesis of nucleic acid analogs, such as gaseous, aqueous ammonia, or primary amines (methylamine, propylamine, tert-butylamine, etc.). Alternatively, the amine can be protected as benzyl carbamate (benzyl chloroformate, Cbz, or Z protection group), or as 9-fluorenylmethyl carbamate (Fmoc protection group).

### Dye Synthesis and Accessibility

Single, high-yielding reaction from inexpensive and commercially available Rhodamine 800 perchlorate dye may be used as a starting material (fluorophore with julolidine core structure, CAS No. [137993-41-0]) along with a primary amine.

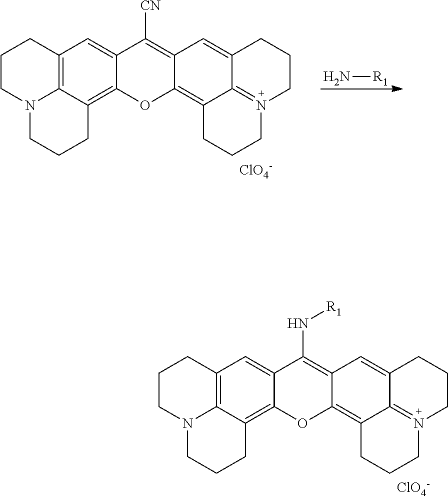

Compounds 1b, 1c, 1h-1j, 1k, 1o, 1r, and 1s (see Tables 1a to 1c) can be used for bio-molecular labeling, either by in situ activation of the carboxylic acid, or by the corresponding NHS-esters. The NHS-esters are prepared by using trifluoroacetic anhydride (TFAA) and N-hydroxysuccinimide (NHS) in the presence of a base:

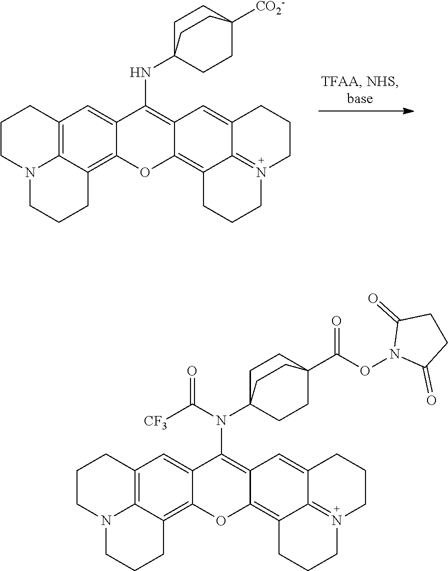

The alcohol function of Compounds 2 h, 2j, 2m, and 2q (see Tables 2a-2c) can be directly converted to the corresponding phosphoramidite for 5′-modification of nucleic acids and nucleic acid analogs:

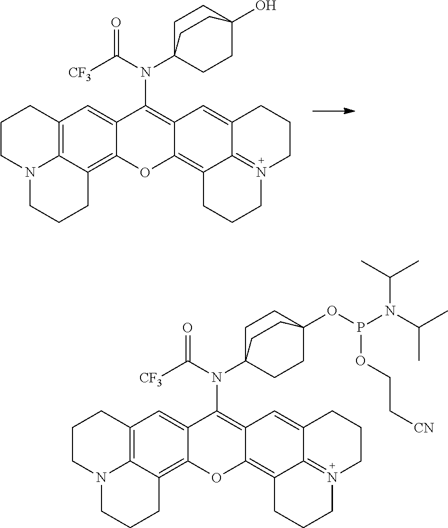

The NHS esters of Compounds 2b, 2c, 2h-1j, 2k, 2o, 2r, and 2s (see Tables 2a-2c) can be converted to the corresponding phosphoramidites for internal modification of nucleic acids and nucleic acid analogs according to the following synthesis sequence:

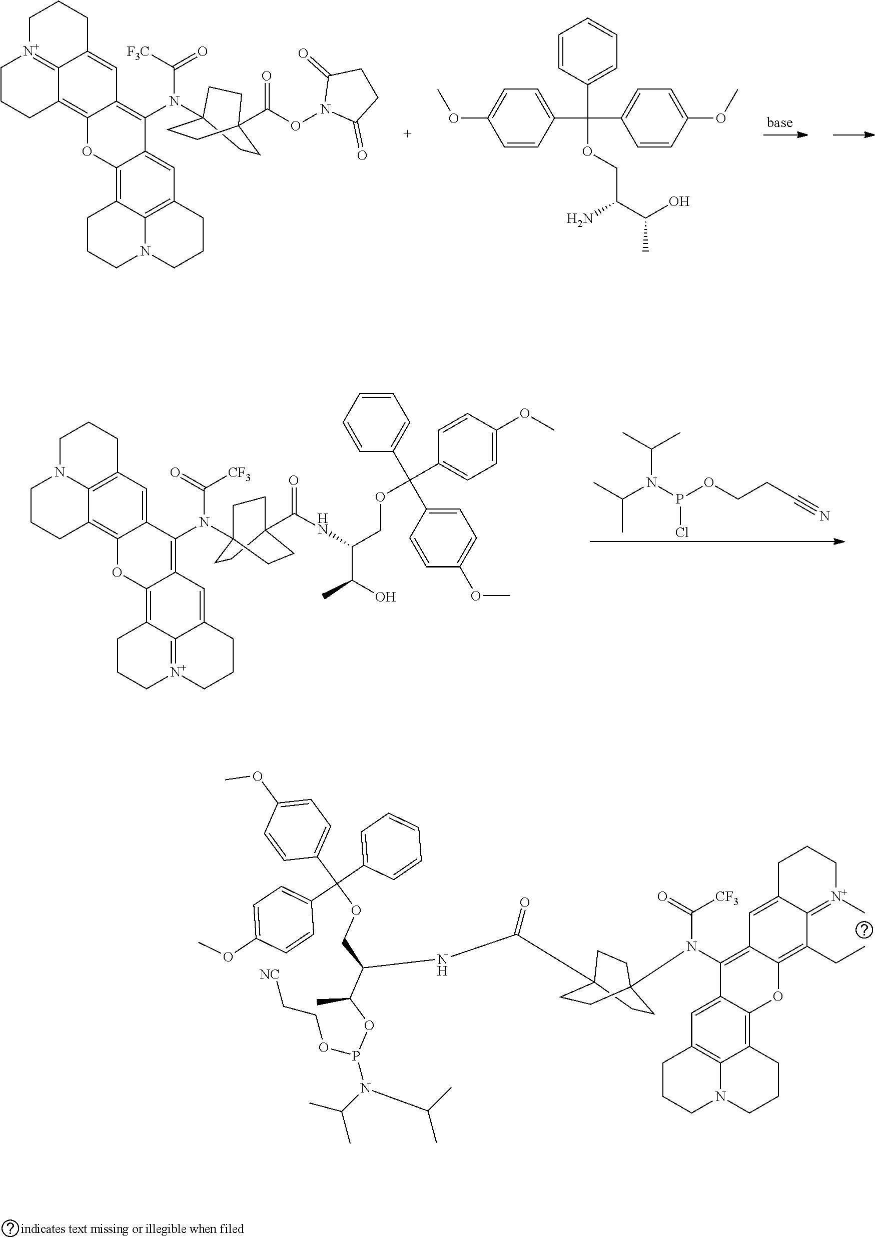

Compounds 1g and 2g (see Tables 1a and 2a) find further use in copper-catalyzed click-chemistry [Cu(I)-catalyzed azide-alkyne 1,3-dipolar cycloaddition, CuAAC]. While Compound 1g can be used for solution labeling of biomolecules, Compound 2g allows for the introduction of the alkyne group during solid-phase synthesis for on-column labeling.

### Results

The absorption and fluorescence maxima, as well as the brightness of Compounds 1a-1s, were analyzed in reference to compound having Formula (1A) where R² is butylamine. The results are set forth in Table 3, herein. It was unexpectedly discovered that the configuration of the carbon that is directly bound to the amino-group in R¹ has a profound influence on the fluorescent properties of the dye core. The dye absorption maximum varied by up to about 106 nm and the fluorescence varied by up to about 35 nm, corresponding to a Stokes shift of about 11 nm to about 92 nm.

A common problem of regular fluorophores with “small” Stokes shift is internal quenching of fluorescence. Such self-quenching is caused by spectral overlap of excitation and emission, and especially prevalent at high fluorophore concentrations. LSS dyes, such as those described herein, generally have better separated spectral bands, which minimizes the reabsorption of photons.

It is believed that there is a non-zero probability for excitation of fluorophores outside of their major excitation peak. As a consequence, the fluorescence from a dye inevitably contributes to the total light detected in multiple emission channels. This spectral “crosstalk” or “bleed-through” can to some extent be compensated for computationally, by using predetermined correction factors. Additionally, scattering of excitation light adds to the background fluorescence in neighboring channels. The dyes of the present disclosure (e.g., those having Formula (IA)) allow a reduction in or even the elimination of crosstalk and scattering from other fluorophores. The dyes of the present disclosure (e.g., those having Formula (IA)) are especially useful in experimental settings where many fluorophores generate a strong background signal. Large spectral separation as for the dyes of the present disclosure (e.g., those having Formula (IA)) allows for more effective filtering of the excitation light, thereby enhancing the sensitivity of target detection (see, e.g., FIG. 2).

It is also believed that the dyes of the present disclosure (e.g., those having Formula (IA)) provide access to fluorescence data from previously inaccessible optical channels. Facilitated by their broad spectral separation and when used in combination with standard fluorophores, the dyes of the present disclosure allow for an increase in the multiplexing capabilities of fluorometric PCR devices by adding further channels in established four- to six-color instruments (see FIG. 2). In principle 21 channels are available from the filter combinations of a six-color instrument. However, in practice the number of channels is limited by the commercial availability of with suitable spectral properties and sufficiently large Stokes shift.

### Activating a Dye and Coupling the Activated Dye to a DNA Molecule

The present disclosure also provides a method of activating a compound of Formula (I) and subsequently coupling the activated compound to an oligomer. The schematic which follows illustrates the solution labeling of a DNA molecule with DMT-MM.

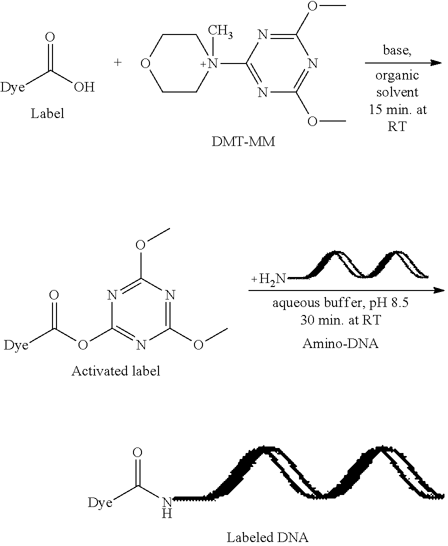

where “Dye” is

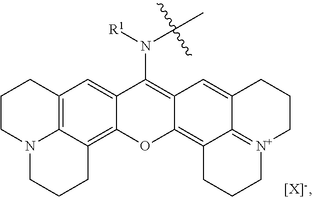

### Synthesis of a TAGS Probe

The present disclosure also provides for methods of synthesizing TAGS probes, where the TAGS probes include compounds having Formula (I) and which are thermostable up to about 100° C. Uses of such TAGS probes are described herein and in U.S. Pat. Nos. 11,028,433, 11,034,997, and 11,345,958; and in United States Patent Publication No. 2021/0269857, the disclosures of which are hereby incorporated by reference herein in their entireties.

In some embodiments, a probe (such as one having Formula (V)) is synthesized by first preparing a 5′-N₃-modified DNA:

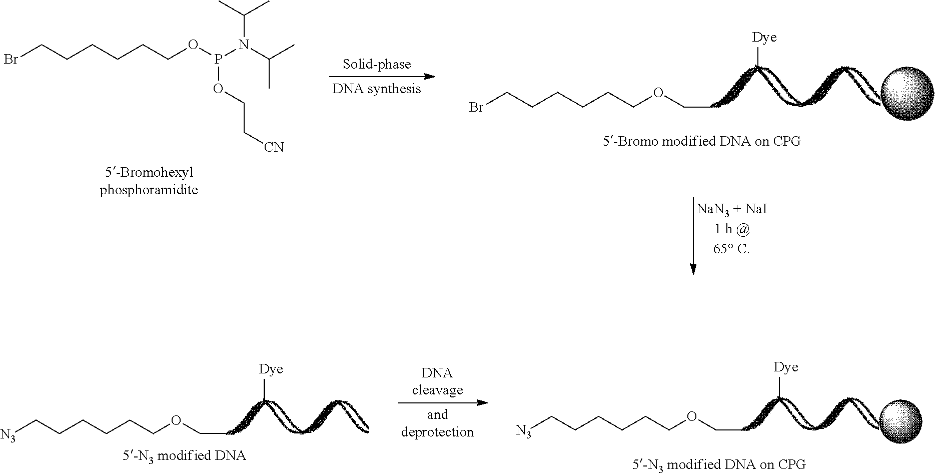

The 5′-N₃-modified DNA is then coupled to an oligonucleotide including a quencher and a first reactive group, such as a reactive group capable of participating in a “click chemistry” reaction (e.g., DBCO). The 5′-N₃-modified DNA is then “clicked” into place when reacted with the oligonucleotide including a quencher and a first reactive group, to provide the probe illustrated of Formula (V).

The present disclosure also provides for methods for directly coupling an oligonucleotide having a terminal amine group to a cyano moiety present at a meso-position of a dye core such as to provide any one of the compounds having Formulas (VIIA) or (VIIB) (see, e.g., Example 6, herein). In some embodiments, a linker is present between the terminal amine group and the oligonucleotide. In some embodiments, the linker is a C₁-C₈ branched or unbranched alkyl, branched or unbranched heteroalkyl, or cycloalkyl group having one or more substituents (e.g., -Me, -Et, —CO₂—, —C₂—CO₂—, -D, or a halogen).

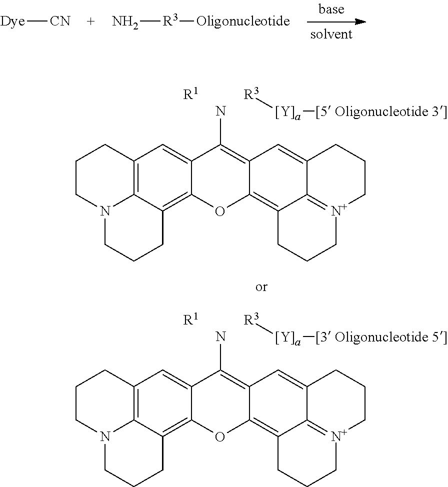

where
Dye-CN represents a dye having a cyano moiety at a meso-position of the dye core;
R³ is a C₁-C₈ alkyl, heteroalkyl, or cycloalkyl group which is substituted with one or more of -Me, -Et, —CO₂—, —C₂—CO₂—, -D, or a halogen; and
Oligonucleotide is an oligonucleotide having between about 5 and about 60 mer.

In some embodiments, the base is N,N-diisopropylethylamine (DIPEA), cesium carbonate, potassium carbonate, sodium carbonate, tributylamine (TBA), N,N-dicyclohexylmethylamine, 2,6-di-tert.-butylpyridine, 1,8-diazabicyclo[5.4.0]undec-7-ene (DBU), 1,5-diazabicyclo[4.3.0]non-5-ene (DBN), 1,1,3,3-tetramethylguanidine (TMG), or 2,2,6,6-tetramethylpiperidine. In some embodiments, the solvent is dimethylsulfoxide (DMSO), sulfolane, N-butylpyrrolidone, γ-valerolactone, 6-valerolactone, N-methylpyrrolidone, N,N-dimethylformamide, sulfolane, or cyrene. In some embodiments, the reaction is performed at a temperature ranging from between about 20° C. to about 70° C. In some embodiments, the reaction is performed for a time period ranging from between about 60 min. to about 72 h. In some embodiments, the Dye is Rhodamine. In some embodiments, the Dye is Rhodamine 800.

### EXAMPLES

The following examples are given to illustrate embodiments of the present disclosure as it is presently preferred to practice. It will be understood that the examples are illustrative, and that the disclosure is not considered as restricted except as indicated in the appended claims.

### Abbreviations

AU=absorbance unit; COU=coumarin; CPG=controlled pore glass; dATP=2′-deoxyadenosine 5′-triphosphate; dCTP=2′-deoxycytidine 5′-triphosphate; dGTP=2′-deoxyguanosine 5′-triphosphate; DBCO=dibenzocyclooctyne modification; DCM=dichloromethane; DIPEA=N,N-diisopropylethylamine; DMSO=dimethyl sulfoxide; DMT-MM=4-(4,6-dimethoxy-1,3,5-triazin-2-yl)-4-methylmorpholinium salt; dUTP=2′-deoxyuridine 5′-triphosphate; EDTA=ethylenediaminetetraacetic acid; eq.=molar equivalents; EtOH=ethanol, EU=emission unit; FAM=fluorescein; HAA=hexylammonium acetate buffer, HCl=hydrochloride salt; HEX=hexachloro-fluorescein; LSS=large Stokes shift; MeCN=acetonitrile; n.d.=not determined; qPCR=real-time polymerase chain reaction; RT=room temperature; R800=rhodamine 800; SPE=solid-phase extraction; TE=Tris/EDTA mixture; TEA=triethylammonium; TEAA=triethylammonium acetate; TEAB=triethylammonium bicarbonate; UPLC-MS=ultra-performance liquid chromatography coupled to a mass spectrometer.

### General Materials and Methods

R800 perchlorate dye [137993-41-0] and 1-bicyclo[1.1.1]pentylamine hydrochloride were obtained from MilliporeSigma (Burlington, MA, U.S.A.). trans-4-Aminocyclohexane-carboxylic acid was obtained from TCI America (Portland, Oregon, U.S.A.). d₉-Butylamine was obtained from C/D/N Isotopes Inc. (Pointe-Claire, QC, Canada). 3-Aminobicyclo[1.1.1]pentane-1-carboxylic acid HCl was obtained from AA Blocks Inc. (San Diego, CA, U.S.A.). Bicyclo[2.2.2]octan-1-amine HCl was obtained from 1Click Chemistry (Kendall Park, NJ, U.S.A.). 4-Aminobicyclo[2.2.2]octan-1-ol HCl and 4-aminobicyclo[2.2.2]octan-1-carboxylic acid were obtained from Absolute Chiral (San Diego, CA, U.S.A.). Reagents and materials for chemical DNA synthesis were obtained from Glen Research (Sterling, VA, U.S.A.). TEAB buffer was obtained as ready-made solution (1.0 M, pH 8.5) and used without further dilution. TEAA and HAA buffers were prepared from diluting commercially available stock solutions (Glen Research, Sterling, VA, U.S.A.) with water to a final concentration of 100 mM. Other reagents were obtained from MilliporeSigma (Burlington, MA, U.S.A.) unless stated otherwise. Dry solvents over activated molecular sieves for chemical reactions were obtained from Acros Organics (Thermo Fisher Scientific, Waltham, MA, U.S.A.). Solvents for chromatography (HPLC grade) were obtained from MilliporeSigma (Burlington, MA, U.S.A.) or VWR (Radnor, PA, U.S.A.). Ultrapure water was obtained from a Milli-Q® purification system (MilliporeSigma) with a resistivity of at least 18.2 MΩ·cm at 25° C.

Chemical reactions were performed in an Eppendorf ThermoMixer® C (Enfield, CT, U.S.A.). Microwave assisted reactions were performed with a Discover® SP microwave system from CEM (Matthews, NC, USA), equipped with a focused single-mode reaction chamber (2.45 GHz) in heavy-walled glass vials (2.0 mL or 10.0 mL). The reaction temperature was monitored with a built-in IR temperature sensor and kept constant by automatic power control. Microwave assisted reactions were stirred under active cooling with compressed air. Flash column chromatography was performed with an automated flash chromatography system (CombiFlash® Rf⁺ Lumen) from Teledyne-Isco (Lincoln, NE, U.S.A.). Replacement of dye counterions was achieved by standard ion exchange procedures, such as ion-exchange, SPE, liquid-liquid extraction, or precipitation from organic solvent.

DNA oligomers carrying 3′-modifications were synthesized on CPG that was preloaded with spacer C3, phosphate, or BHQ-2 (Black Hole Quencher®). DNA sequences with a primary amino-modification were synthesized by solid-phase DNA synthesis using an amino-modifier phosphoramidite. For 5′-terminal modifications 6-(trifluoroacetylamino)-hexyl-(2-cyanoethyl)-(N,N-diisopropyl)-phosphoramidite was used (5′-amino-C₆). For internal modifications 5′-dimethoxytrityl-5-[N-(trifluoroacetylaminohexyl)-3-acrylimido]-2′-deoxyuridine, 3′-[(2-cyanoethyl)-(N,N-diisopropyl)]-phosphoramidite was used (amino-C-dT). Where necessary due to sequence context, amino-C₆-dT could also be used at the 5′-end. DNA sequences with 5′-azido modification were prepared by synthesizing 5′-bromo-modified DNA with bromo-hexyl phosphoramidite and subsequent bromo to azido conversion on the solid support. Br/N₃-exchange was performed by adding a solution of sodium azide and sodium iodide in DMSO (100 mM each) to the CPG and heating the mixture for 1.0 h at 65° C. (quantitative reaction). Following DNA synthesis and on-column modifications, the DNA was cleaved, deprotected, desalted and precipitated with standard methods. The latter two steps were done to ensure removal of any traces of amines. Aqueous solutions of DNA sequences were dried with a rotary vacuum concentrator (SpeedVac™, Thermo Fisher Scientific Inc., Waltham, MA, U.S.A.) or with a lyophilizer (Labconco freeze dryer with 4.5 L ice capacity and −105° C. collector temperature; Labconco Corp., Kansas City, MO, U.S.A.).

UPLC analyses were performed on a Waters I-class ACQUITY UPLC (Waters Corporation, Milford, MA, USA) equipped with diode array, fluorescence, and mass spectrometry (ZSpray™) detectors. A Waters Oligonucleotide BEH C18 column (130 Å, 1.7 μm, 2.1×50 mm) was used with appropriate gradients of TEAA buffer (100 mM, pH 7.0) against MeCN at 1.0 ml/min. Chromatograms were recorded at 260 nm for DNA and at the absorption maximum of the respective dye or dye-labeled probe. Semi-preparative HPLC purifications were performed on a Waters 600 HPLC with 996 photodiode array detector and Waters XBridge™ BEH C18 OBD Prep column (130 Å, 5 μm, 19.0×250 mm) at 10.0 ml/min. Prior to injection the samples were filtered through a Teflon® syringe filter (0.22 μm). Absorption spectra were obtained with a NanoDrop One© Spectrophotometer (Thermo Fisher Scientific Inc., Waltham, MA, U.S.A.) with background correction at 750 nm. Fluorescence spectra and thermostability of fluorescence data were recorded with a Cary Eclipse Fluorescence Spectrophotometer with Temperature Controller (Agilent Technologies, Santa Clara, CA, U.S.A.).

### Example 1: General Procedure to Prepare Compounds R, 1a-1s

R800 perchlorate dye (1.0 eq., 100 mM in DMSO, 10 μL) in DMSO was converted with primary amines (5.0 eq., 100 mM in DMSO) in the presence of a base in a reaction mixer at 50° C. For n-butylamine, d₆-butylamine, ethanolamine, 2-methoxyethylamine, 2-(2-aminoethoxy)ethanol, propargylamine, tert.-butylamine, and 2-amino-2-methyl-1-propanol the base was DIPEA (1.0 eq.). For amines with carboxylate functionality (glycine, β-alanine, L-alanine, L-leucine, L-valine, L-(+/−)-3-aminobutyric acid, trans-4-aminocyclohexanecarboxylic acid, 4-aminobicylco[2.2.2]octane-1-carboxylic acid) cesium carbonate (2.0 eq.) was used as a base. For amines that were obtained as HCl salt (1-bicyclo[1.1.1]pentylamine HCl, 3-aminobicyclo[1.1.1]pentane-1-carboxylic acid HCl, bicyclo[2.2.2]octan-1-amine HCl) the amount of cesium carbonate was increased to neutralize the acid (5.0 eq.). The identities the dye products were confirmed by mass spectrometry analysis in the positive ion mode.

The spectroscopic properties of the dye products R, 1a-1s were determined by UPLC-MS analysis. A sample of the reaction solution (0.5 μL) was diluted with MeCN (29.5 μL) and separated on a C18 stationary phase using a TEAA-buffered mobile phase and a gradient of MeCN (40-70% MeCN in 2.0 min.). For compound 1p the gradient was extended by 30 s. The absorption and emission spectra were recorded by injecting an amount that gave ˜0.5-1.0 AU at the absorbance maximum of the respective dye.

To determine the fluorescence per unit of absorption as a measure of dye brightness, a sample amount was injected to UPLC that gave ˜0.1 AU at the absorbance maximum of the respective dye. The fluorescence emission was measured by excitation at the absorption maximum (3D fluorescence mode, 1.0 PMT gain, 1.0 s time constant) and a fluorescence emission window of 100 nm centered at the emission maximum. The peak area of the fluorescence peak was divided by the peak area of the absorption peak.

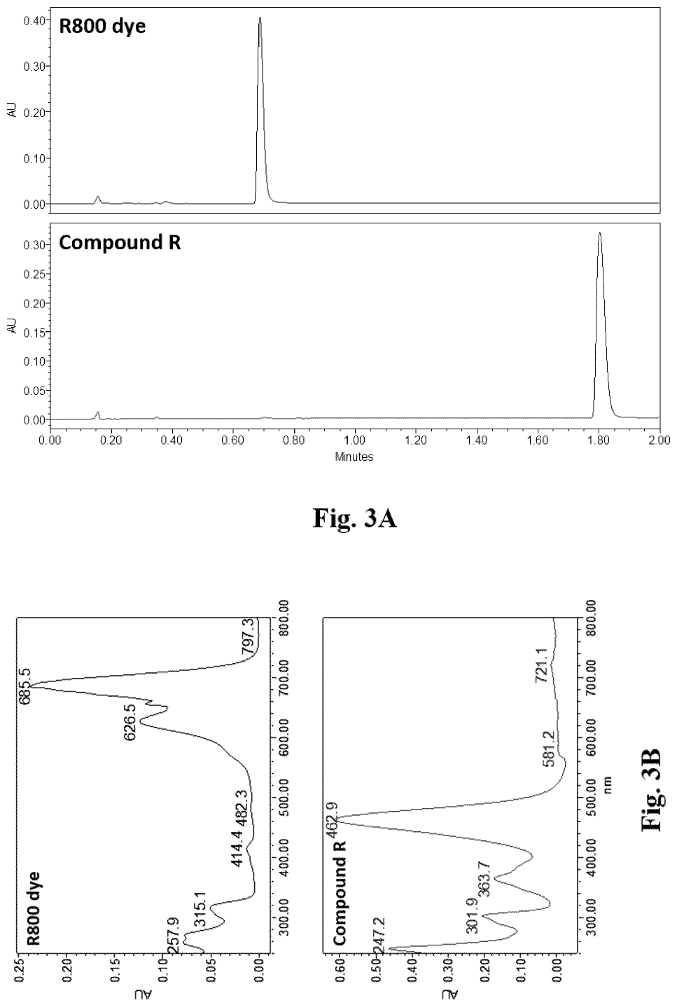

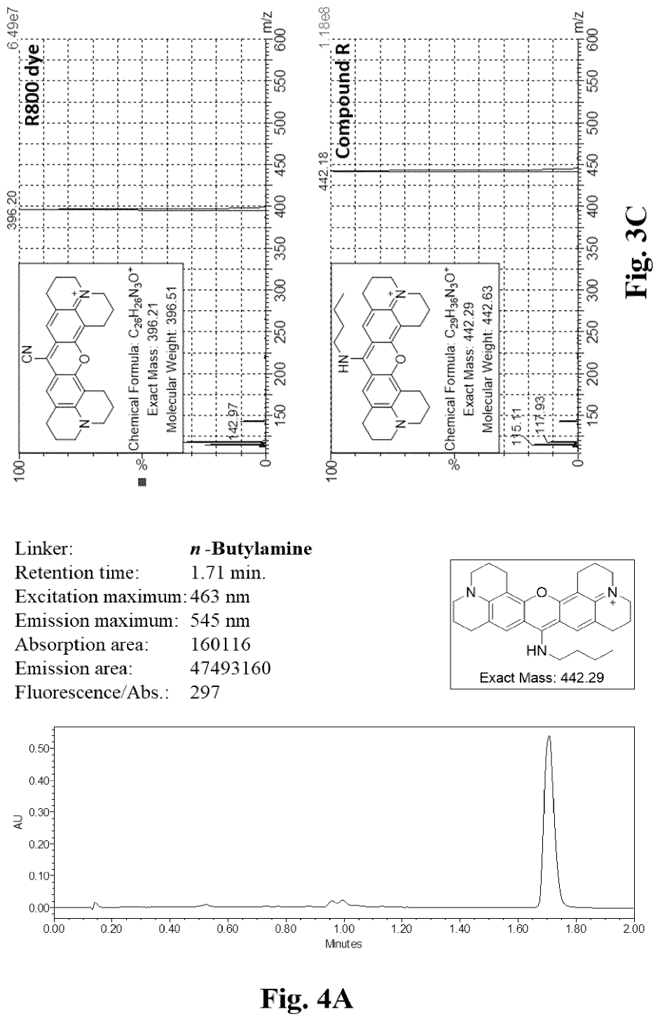

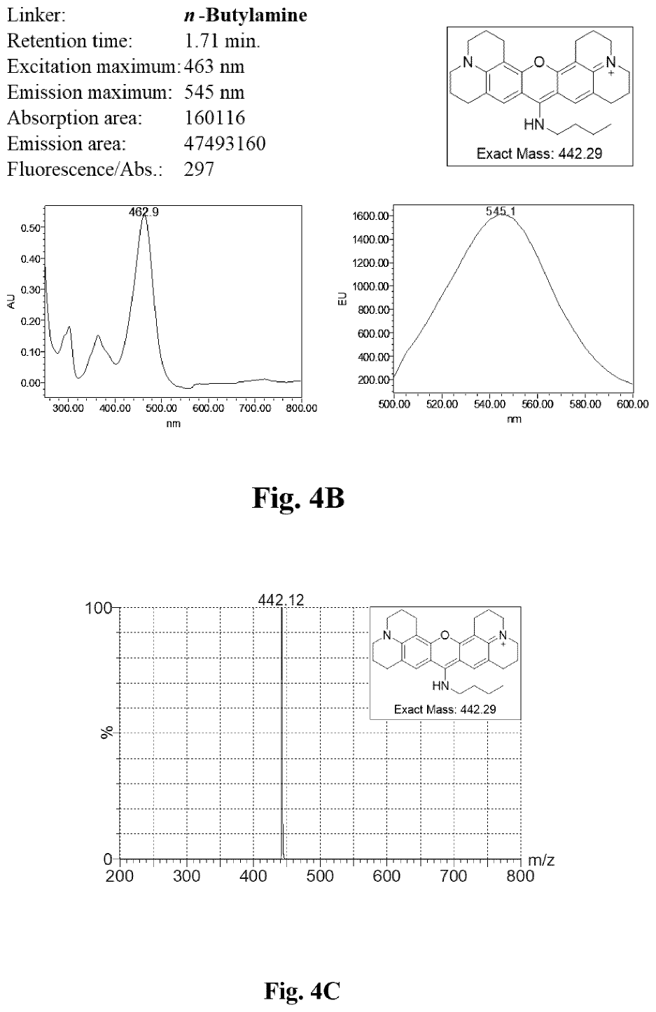

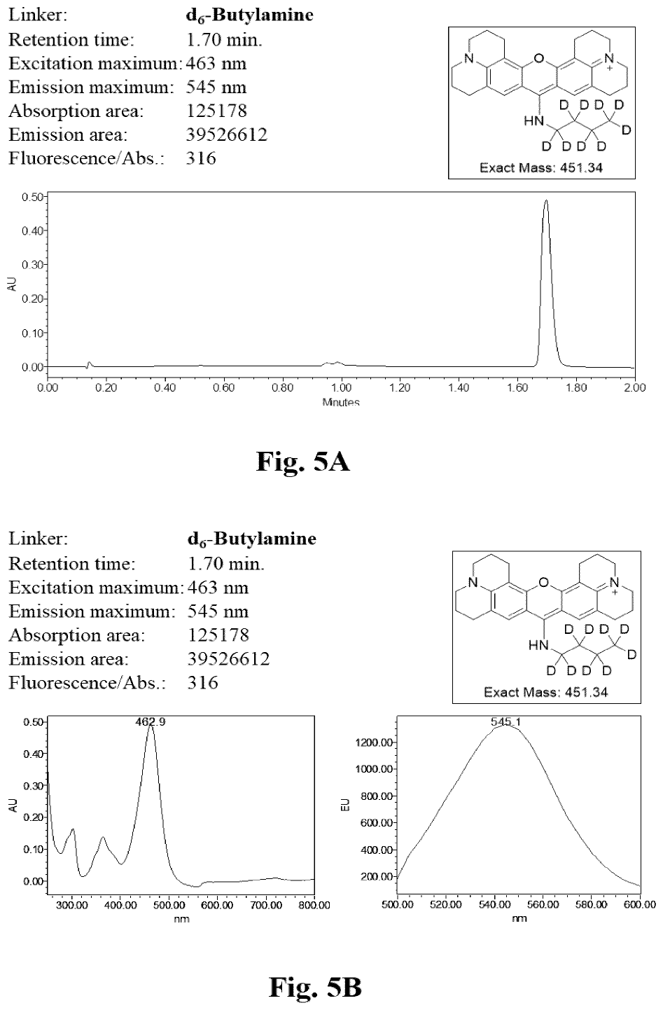

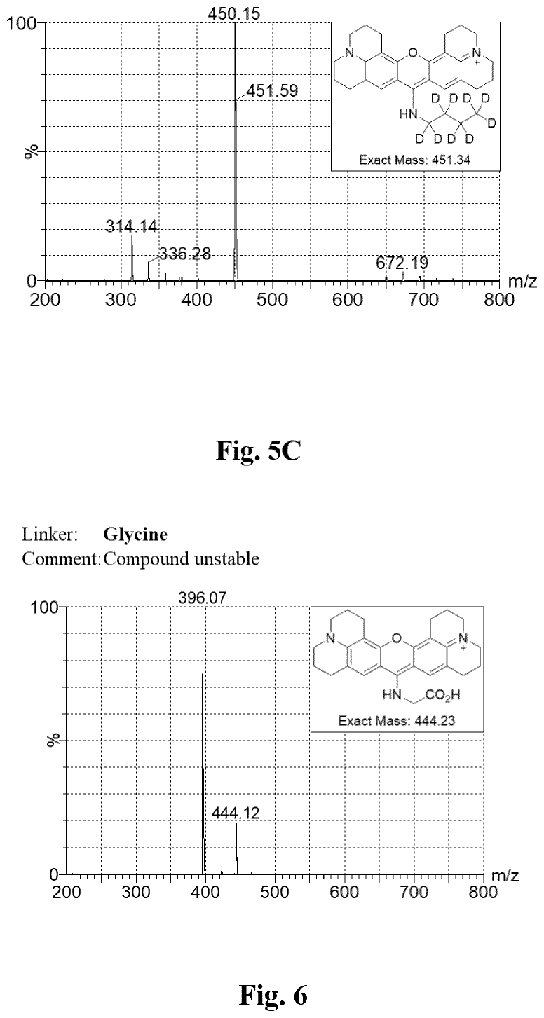

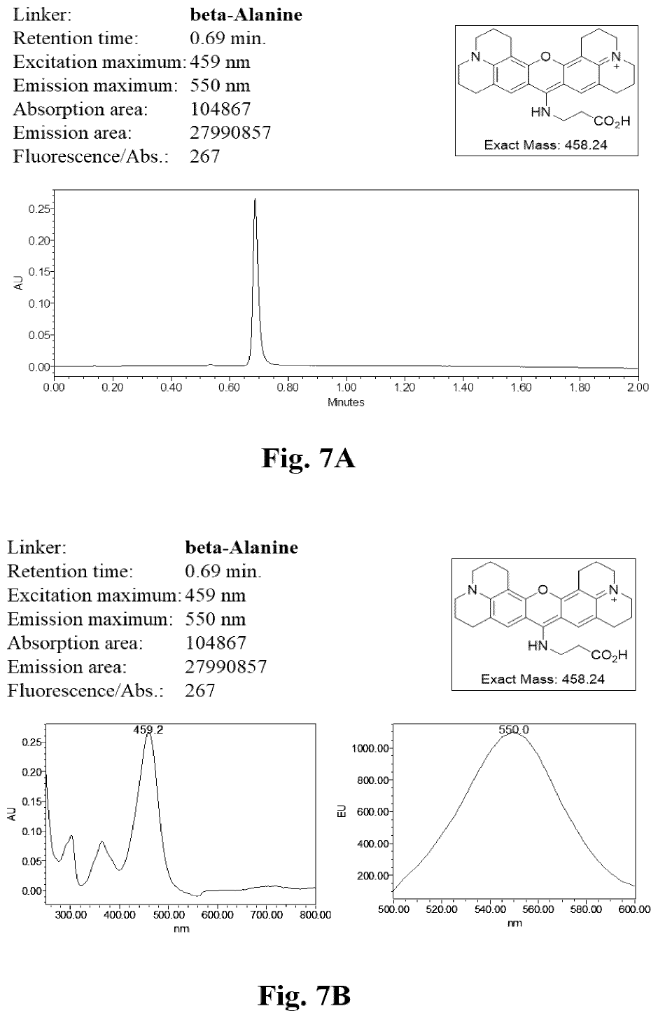

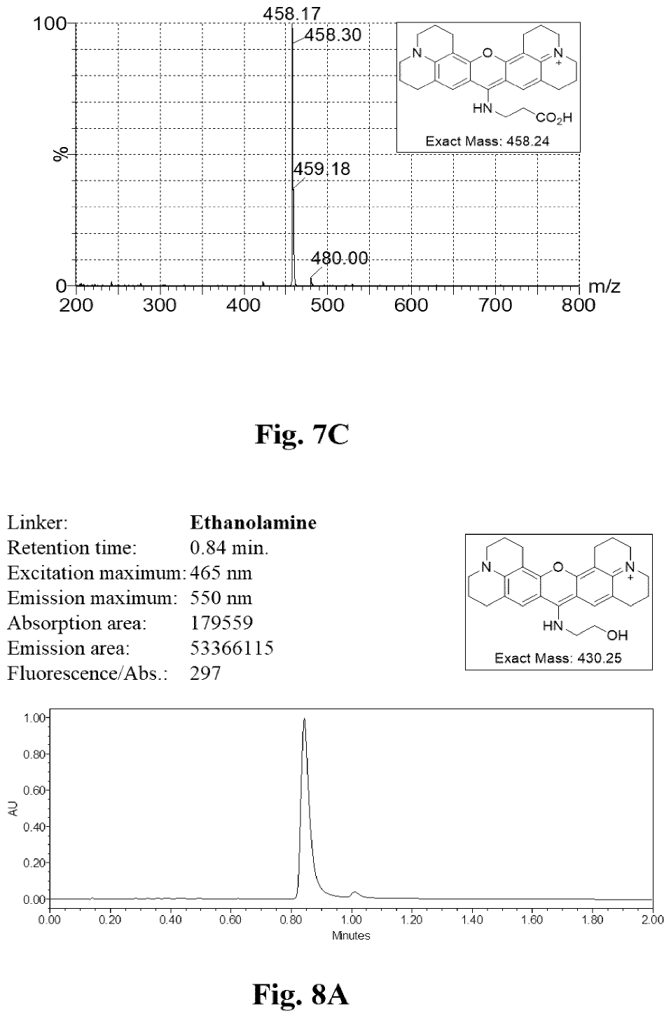

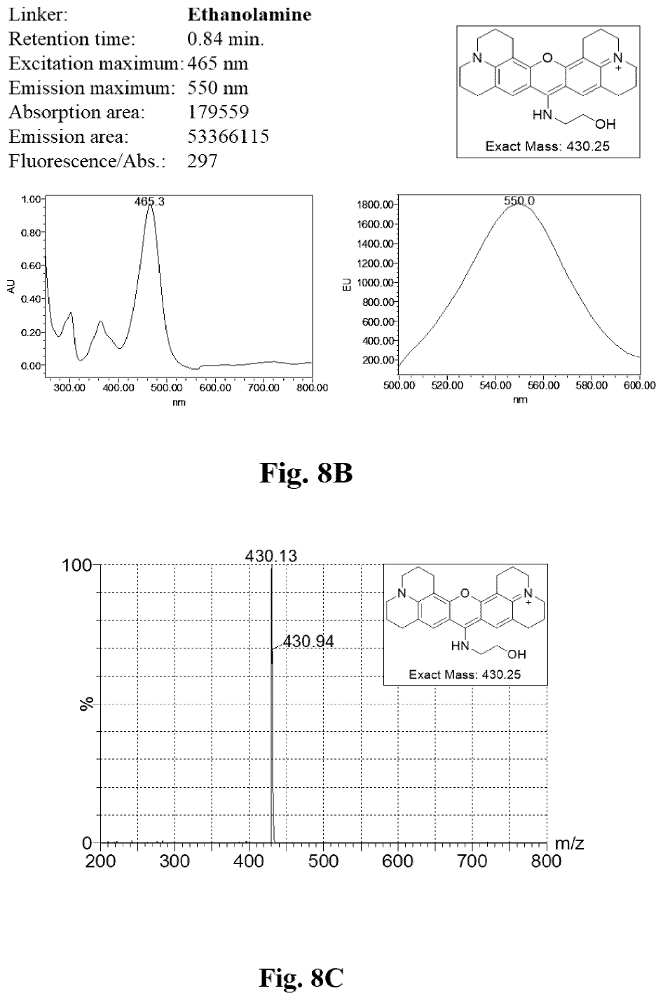

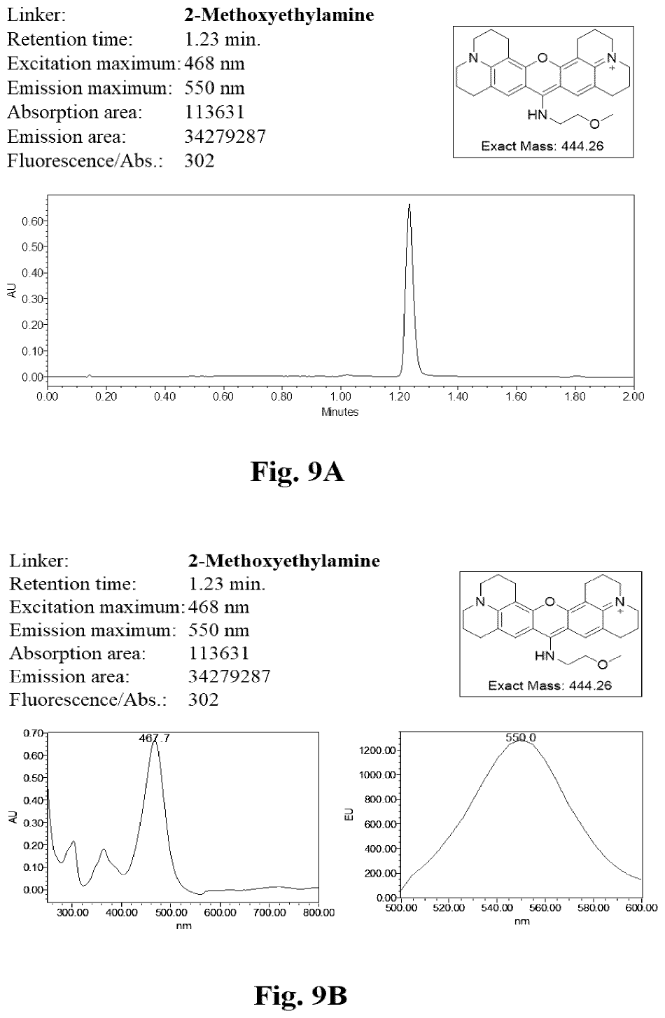

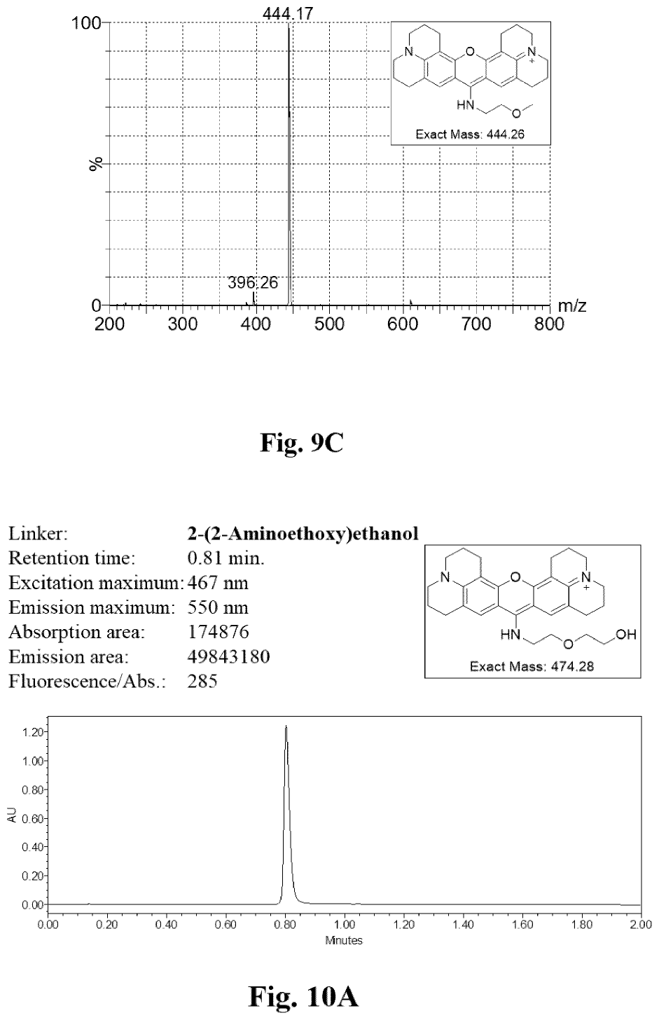

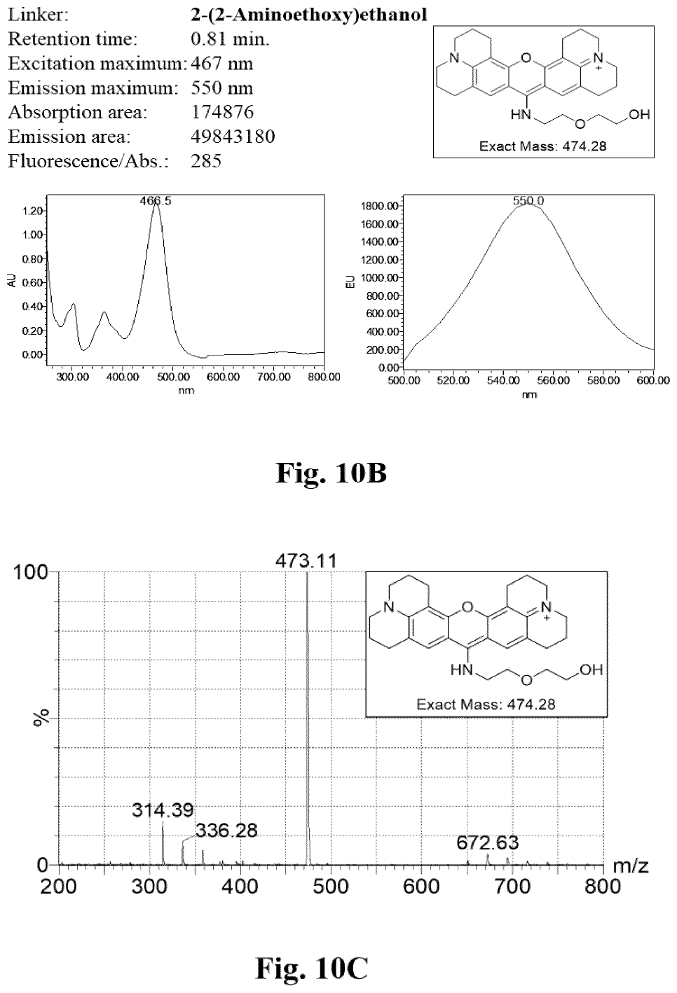

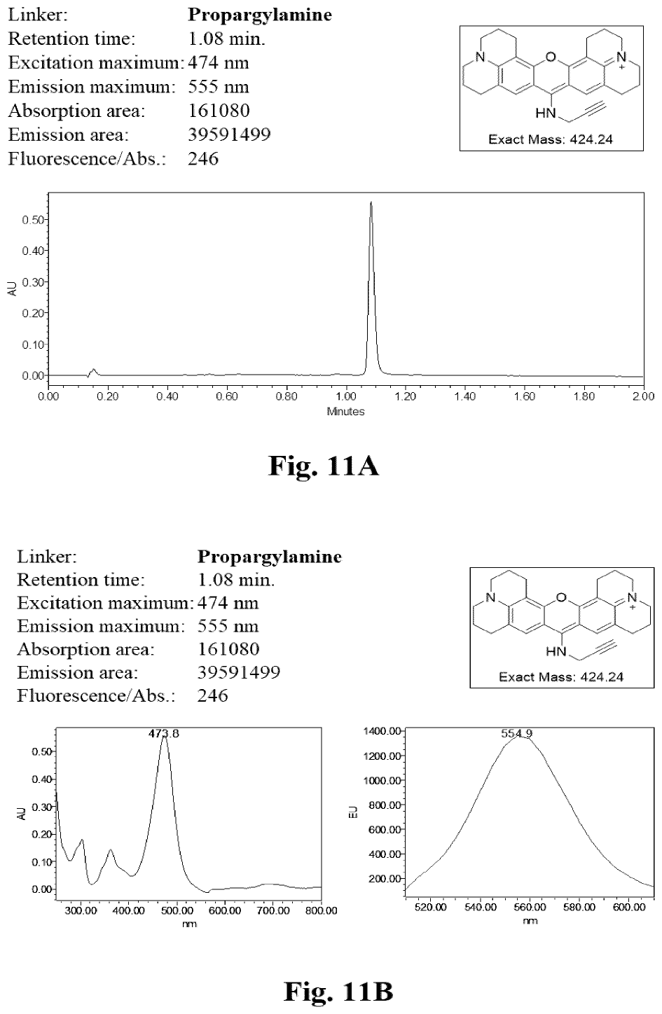

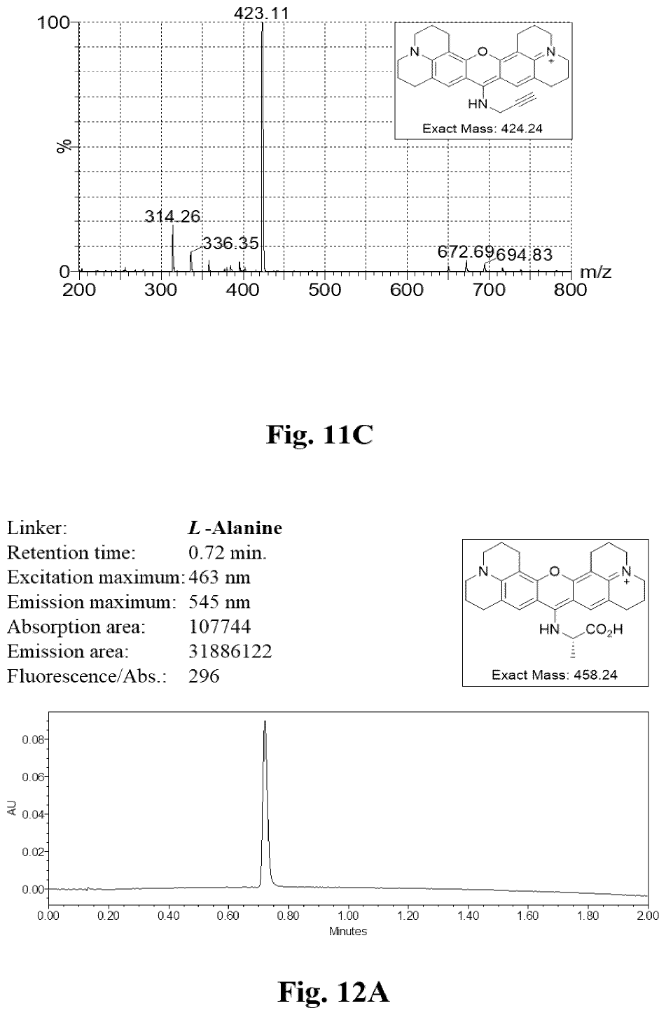

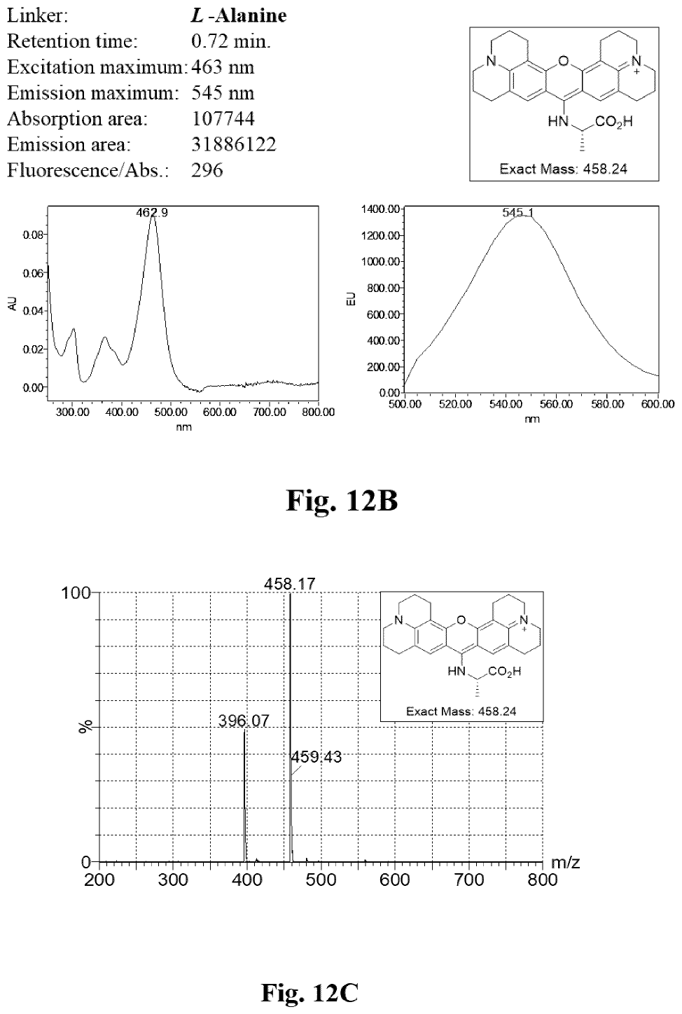

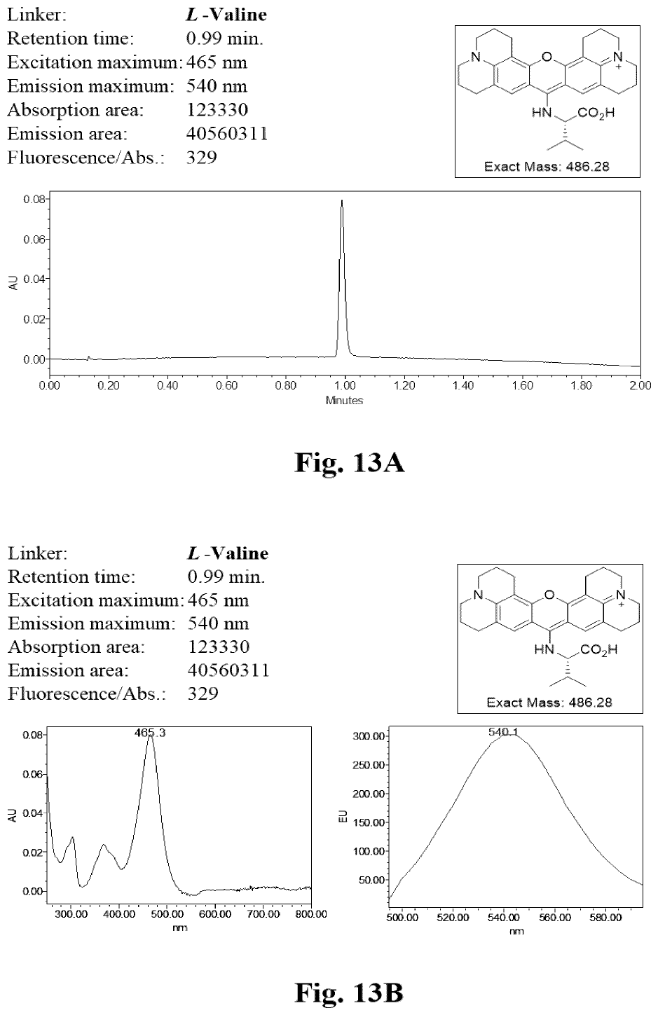

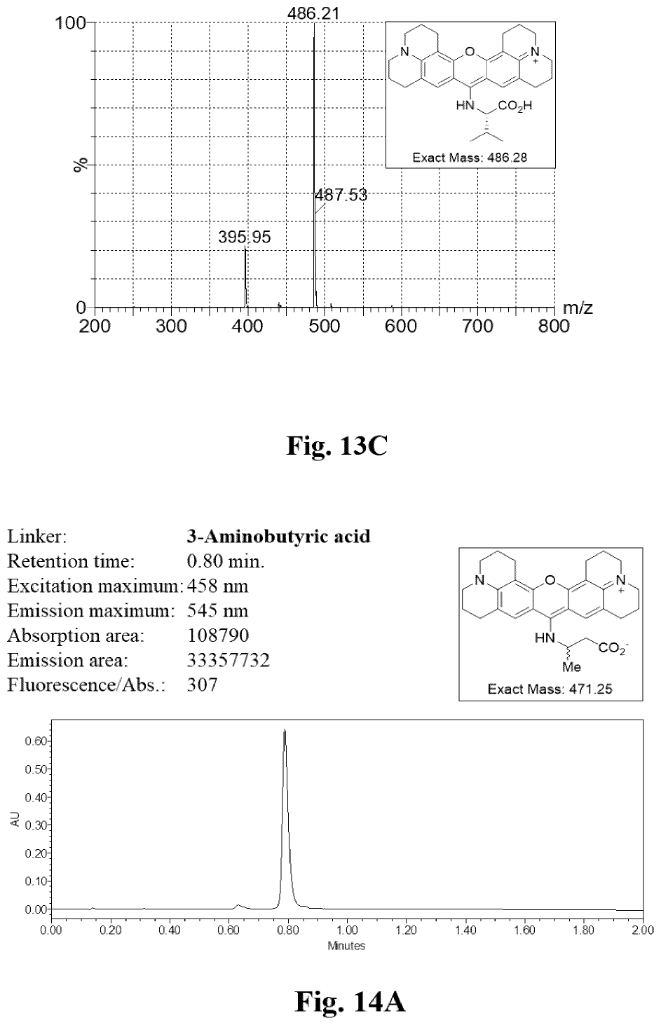

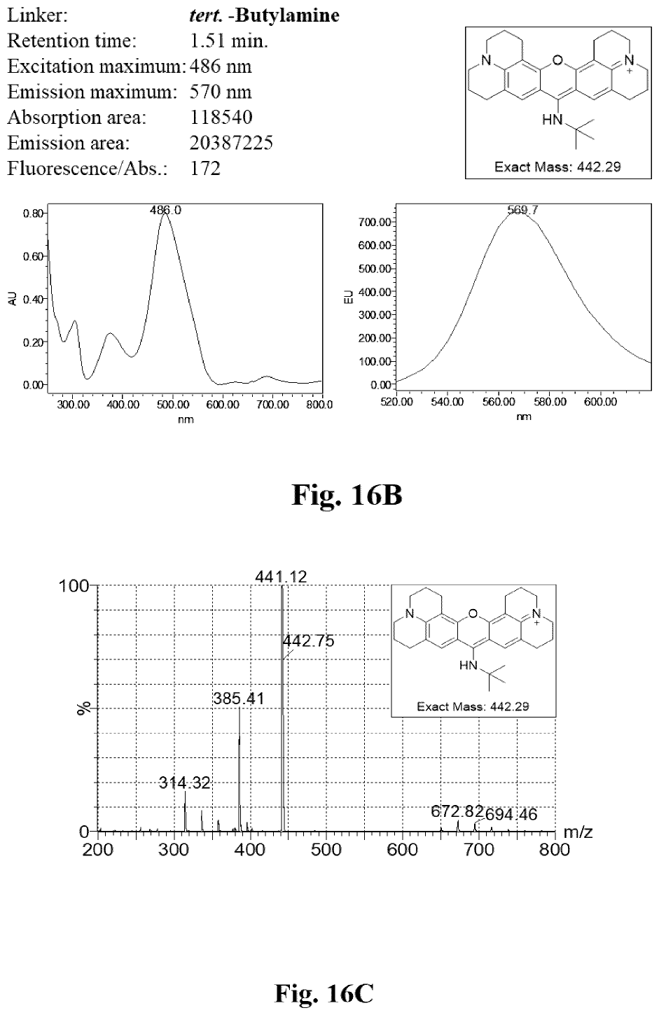

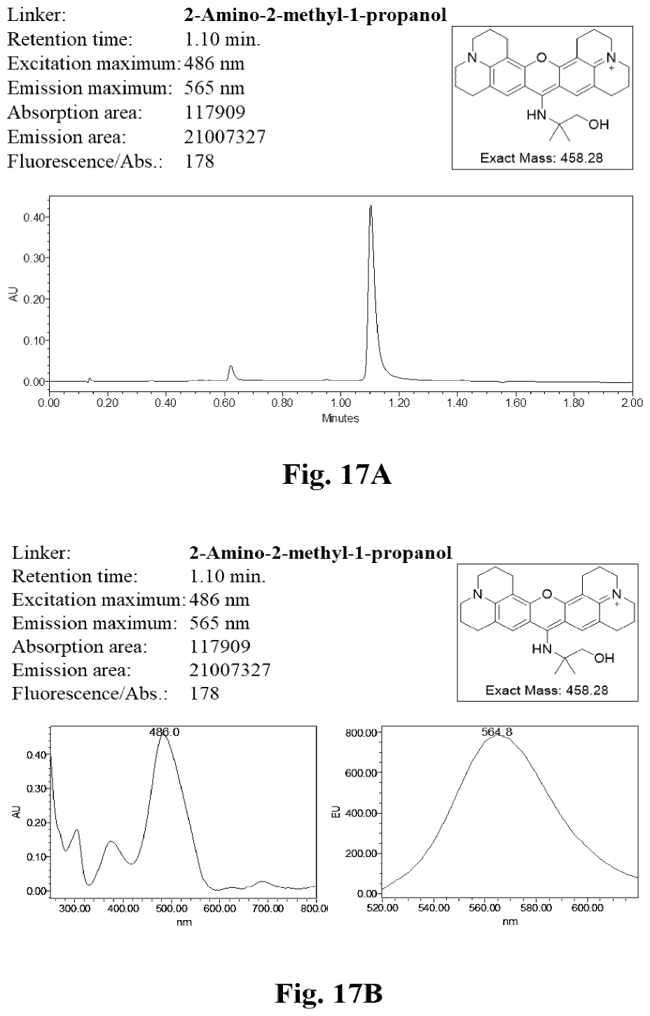

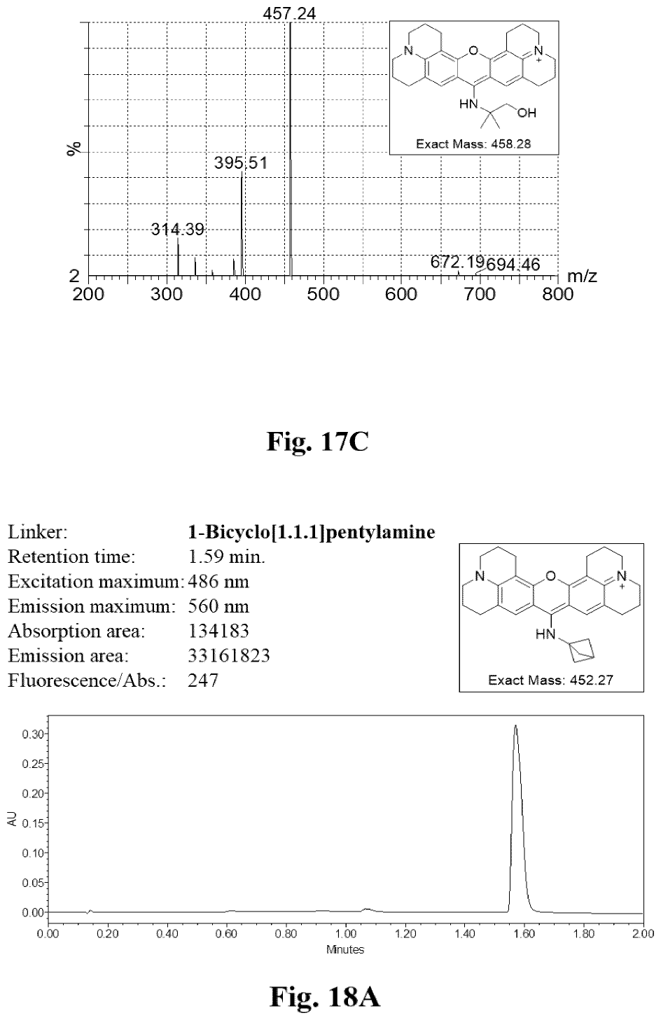

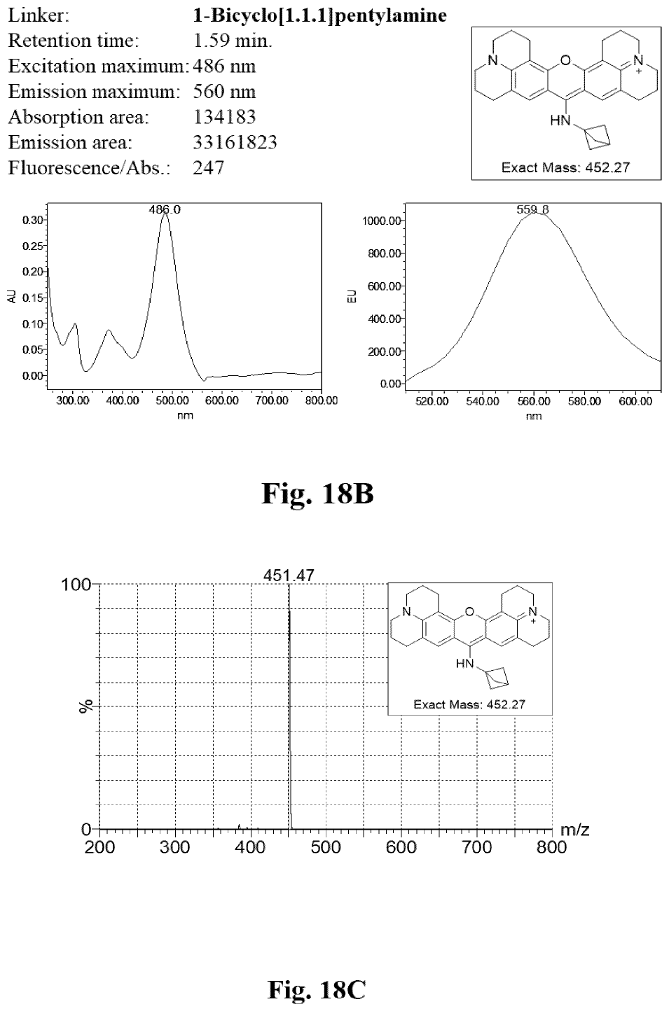

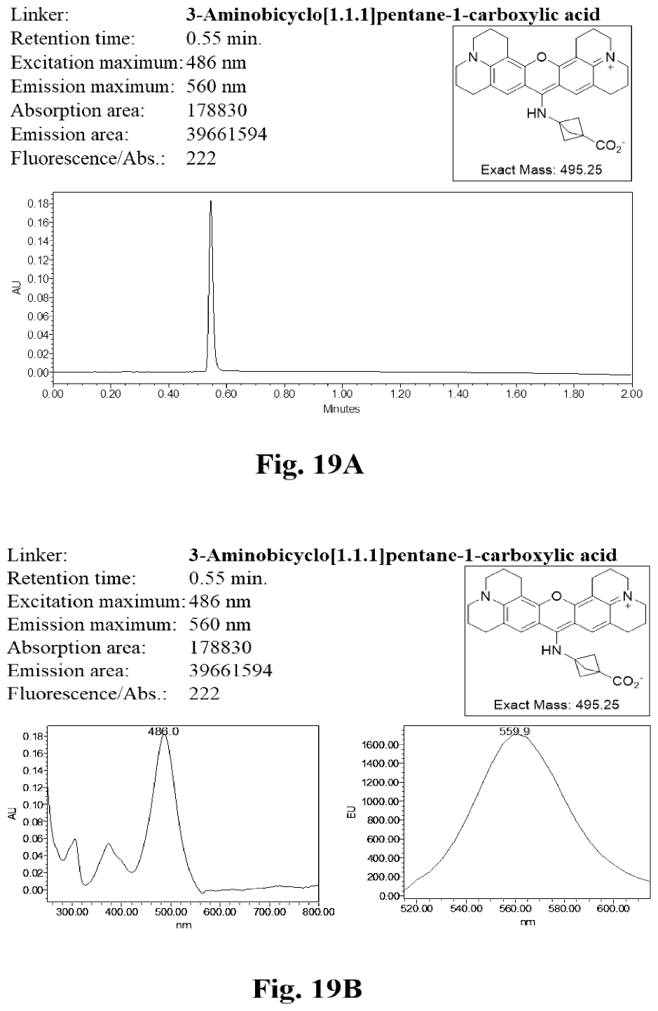

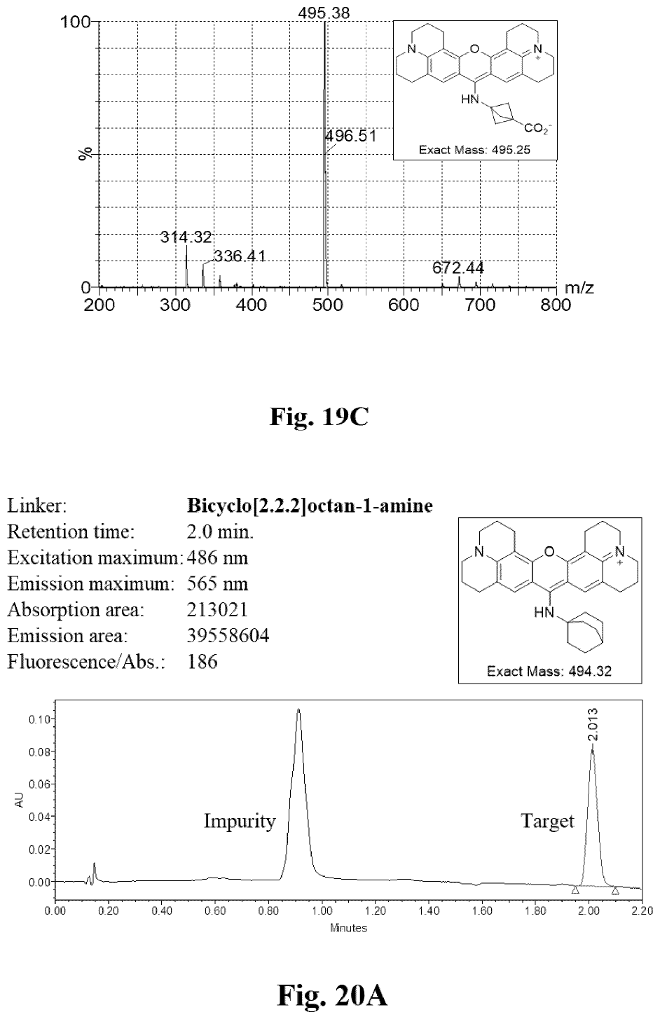

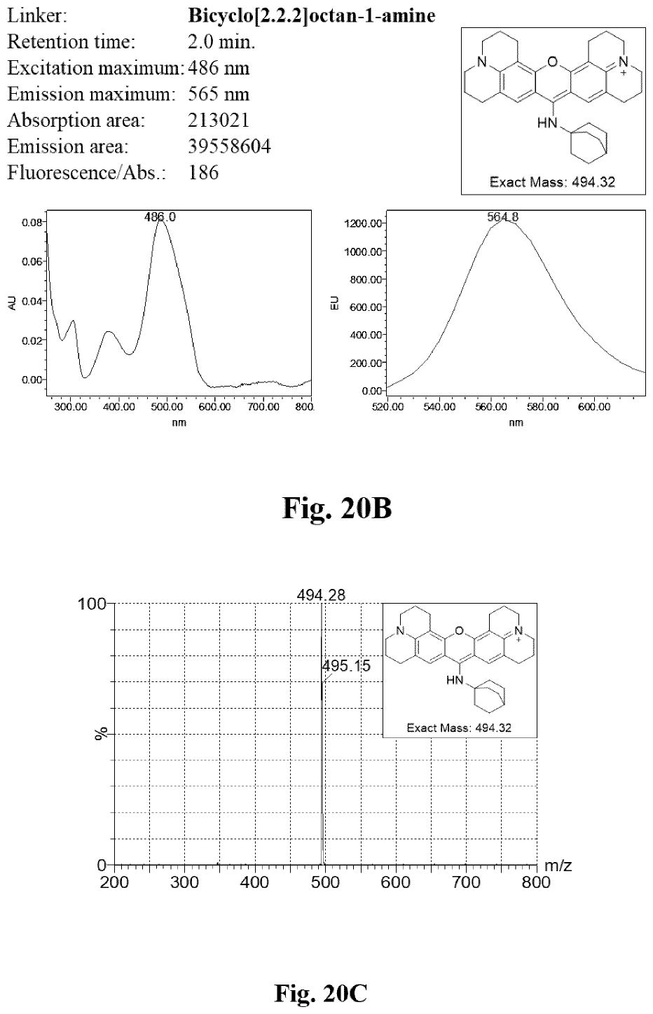

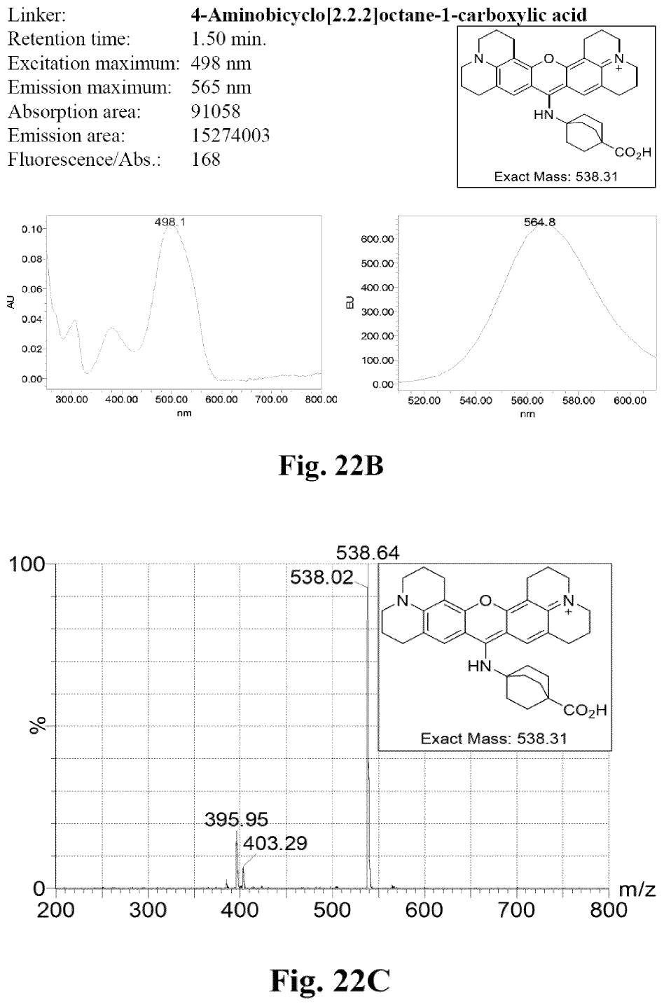

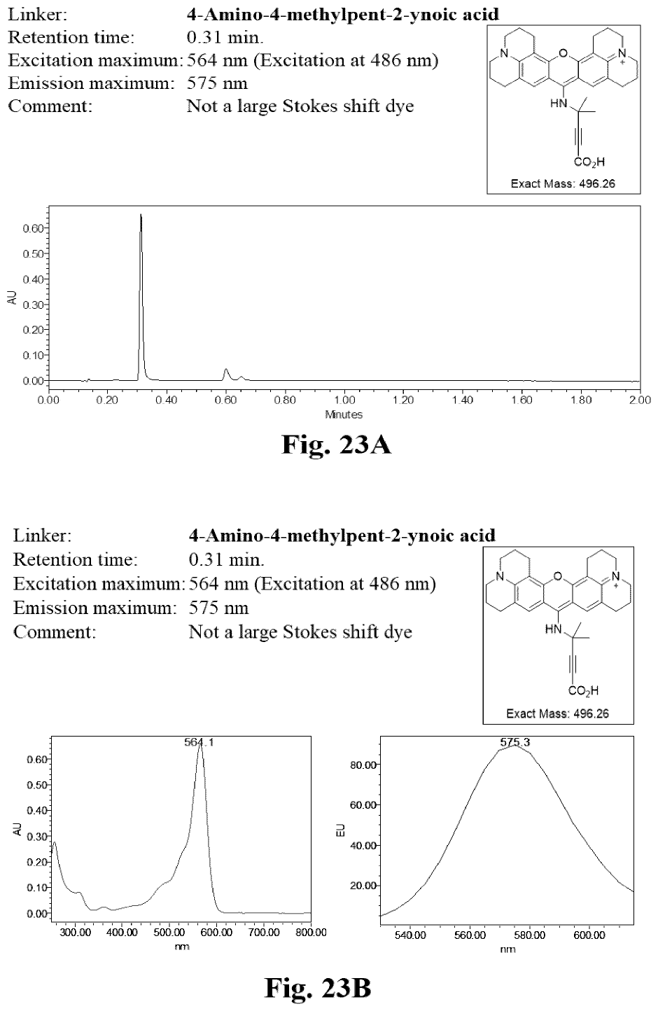

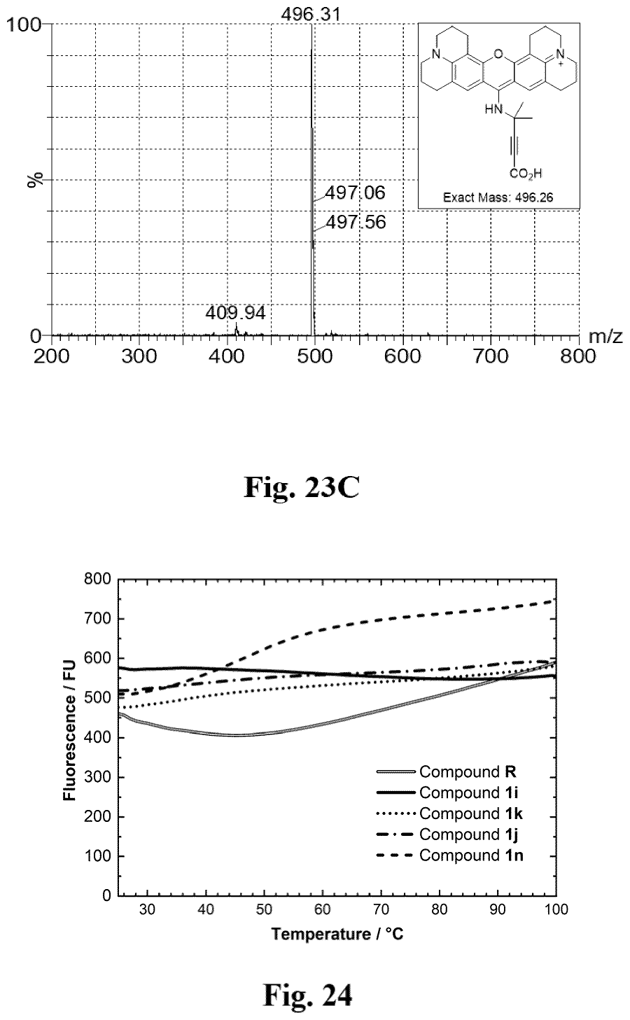

Results: Analytical data for the reaction of n-butylamine with R800 to give compound R are shown in FIGS. 3A, 3B, and 3C. Detailed analytical data for compounds 1a-1s are shown in FIGS. 4-23 (A: Chromatograms; B: Excitation & emission spectra; C: Mass spectra). The spectroscopic data for compounds R, and 1a-1s have been summarized in Tab. 1. Compound 1b was unstable and not analyzed further (FIG. 6). Surprisingly, compound is was not a LSS dye and showed typical spectral features of a “regular” fluorescent dye (˜11 nm Stokes shift, FIG. 23B). This result stands in contrast to compound 1g, which showed a Stokes shift of 81 nm (FIG. 11B). In summary, these results demonstrated that the linker moiety exerted a profound influence on the spectroscopic properties of the dye core.

### Example 2: Synthesis of Compound 1j

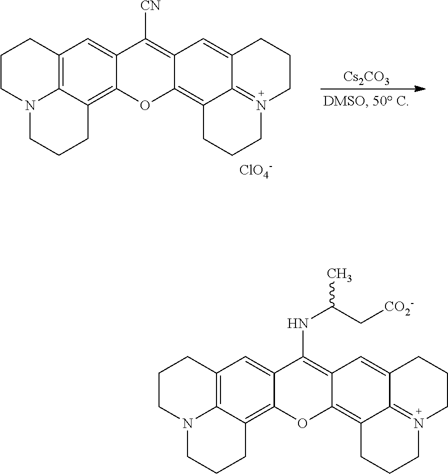

(+/−)-3-Aminobutyric acid (5.0 eq., 151 mol) and cesium carbonate (2.0 eq., 61 mol) were thoroughly mixed in dry DMSO (909 L). R800 perchlorate dye (1.0 eq., 30 mol, 33 mM) was added and the suspension mixed at 50° C., during which the color of the reaction mixture changed from dark blue to yellow-orange. The reaction mixture filtered with a spin filter and the filtrate diluted with an aqueous solution of sodium iodide (0.5 M). The precipitate was isolated by centrifugation, dried at high vacuum, and purified with preparative HPLC. For DNA labeling, the acetate counter ion was exchanged to iodide.

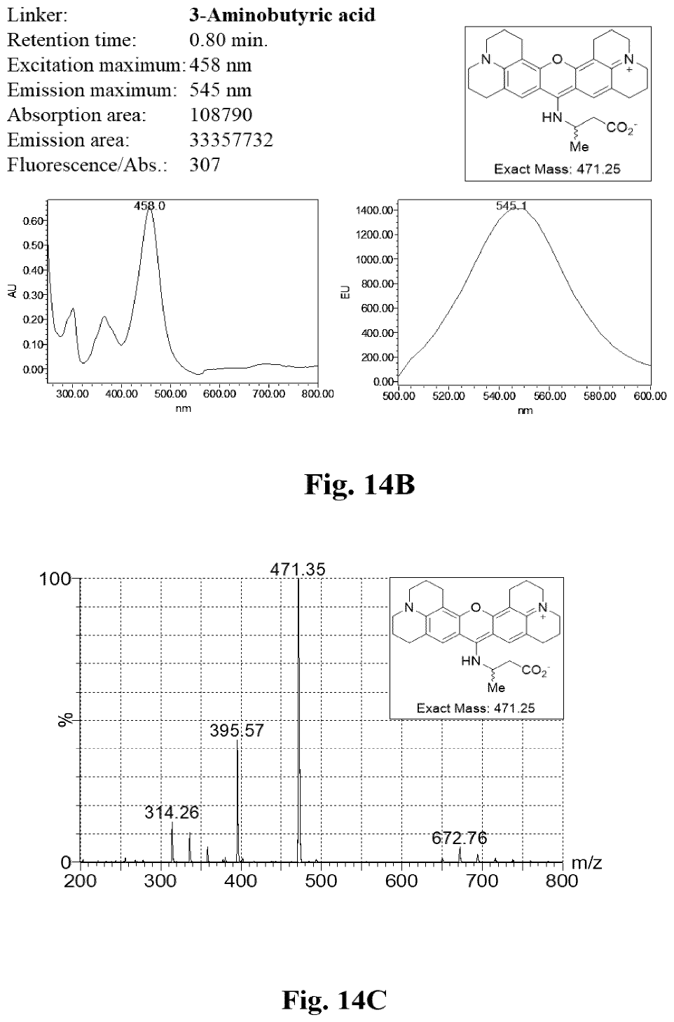

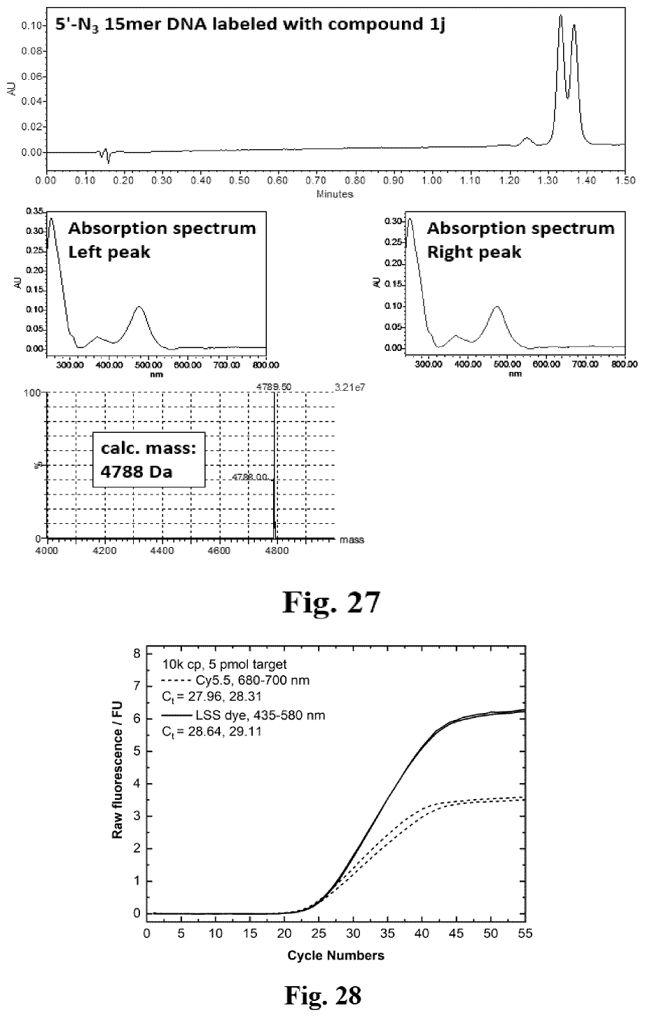

Results: Successful synthesis of compound 1j was demonstrated by the analytical data in FIG. 14 (A: Chromatograms; B: Excitation and emission spectra; C: Mass spectrum). Compound 1j was used for DNA labeling in Example 7 (FIG. 27).

### Example 3: Synthesis of Compound 1k

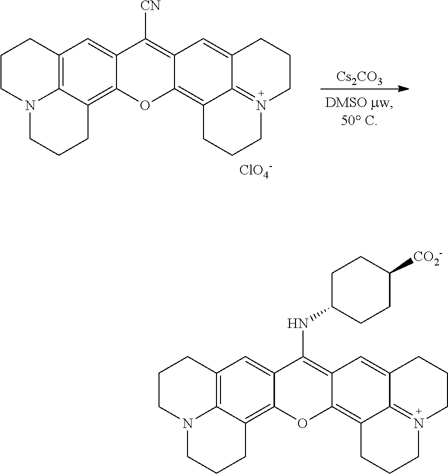

trans-4-Aminocyclohexanecarboxylic acid (2.0 eq., 403 mol) and cesium carbonate (2.0 eq., 403 mol) were thoroughly mixed in dry DMSO (8.1 mL). R800 perchlorate dye (1.0 eq., 403 mol) was added, and the suspension was heated 15 min. at 50° C. with a microwave reactor, during which the color of the reaction mixture changed from dark blue to dark green. The reaction progress was monitored with UPLC-MS. Any undissolved solids were removed by centrifugation. The solution was filtered with a 0.22 μm Teflon® syringe filter and added dropwise to a stirring solution of sodium iodide (0.5 M) in water. The mixture was vortexed and the solid isolated by centrifugation. The residue was dried at high vacuum. Any unreacted R800 starting material and decomposition products were removed by precipitating the dye from DCM at −20° C. The product was dried at high vacuum and lyophilized from MeCN to yield the yellow-orange target compound.

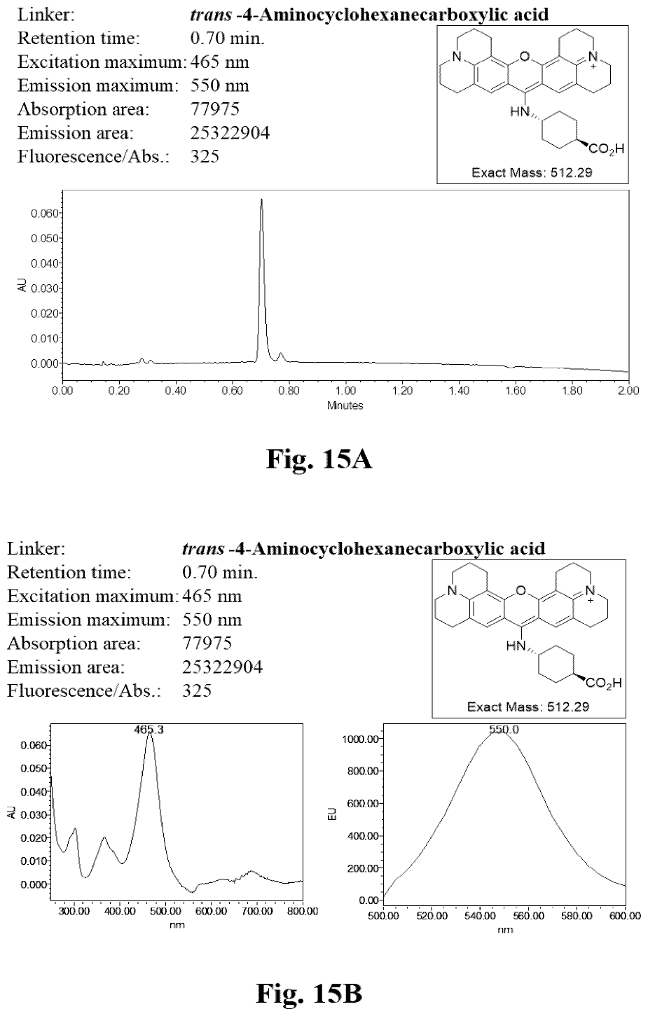

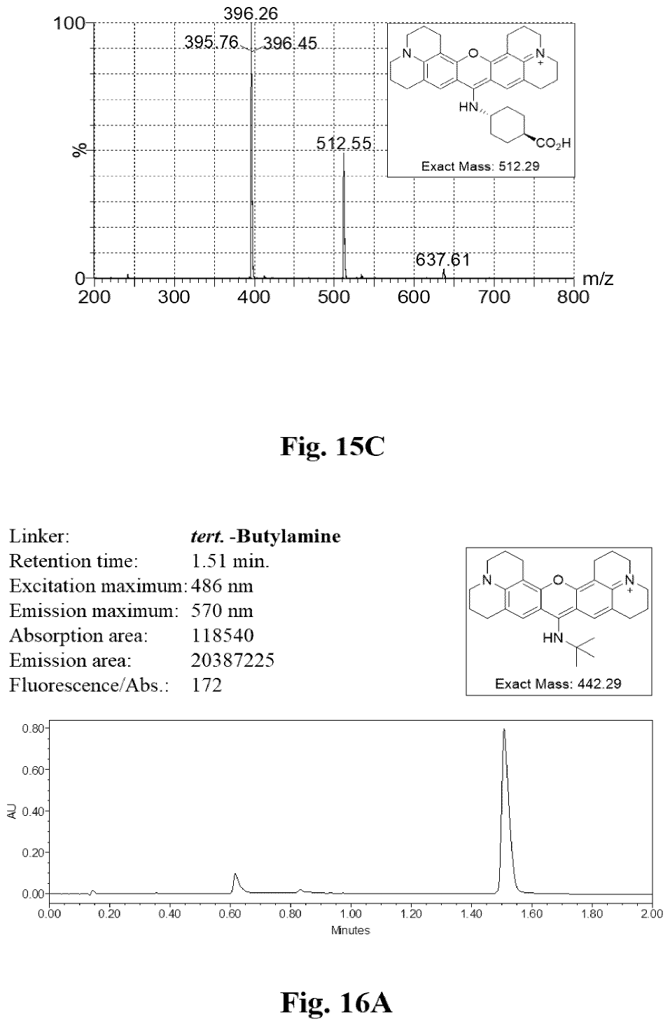

Results: Successful synthesis of compound 1k was demonstrated by the analytical data in FIG. 15 (A: Chromatograms; B: Excitation and emission spectra; C: Mass spectrum).

### Example 4: Synthesis of Compound 1q

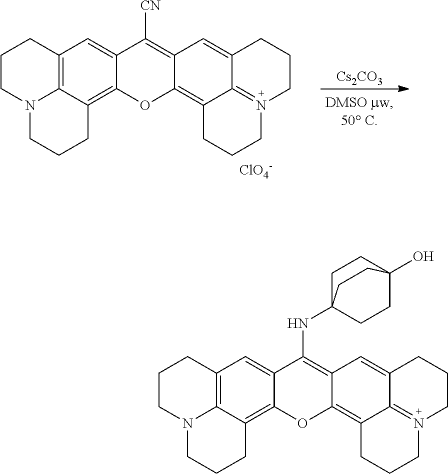

4-Aminobicyclo[2.2.2]octan-1-ol hydrochloride (2.0 eq., 403 mol) and cesium carbonate (2.0 eq., 403 mol) were thoroughly mixed in dry DMSO (8.1 mL). R800 perchlorate dye (1.0 eq., 403 mol) was added, and the suspension was heated 10 min. at 50° C. with a microwave reactor, during which the color of the reaction mixture changed from dark blue to brown-orange. The reaction progress was monitored with UPLC-MS. Any undissolved solids were removed with a 0.22 μm Teflon® syringe filter, and the solution was added dropwise to a stirring solution of sodium iodide (2.0 g) in water (30 mL). The precipitate was vortexed and then isolated by centrifugation. Any unreacted R800 starting material, decomposition products, water were removed by extracting the solid with dry toluene (2×20 mL, or until the supernatant was not colored blue). The residue was dried at high vacuum. Excess salts were removed by dissolving the crude in DCM and filtering off colorless solids. DCM was evaporated at reduced pressure and the solid redissolved in MeCN. The solution was frozen in liquid nitrogen and lyophilized to dryness, giving the bright orange target compound at 20% yield and 85% purity.

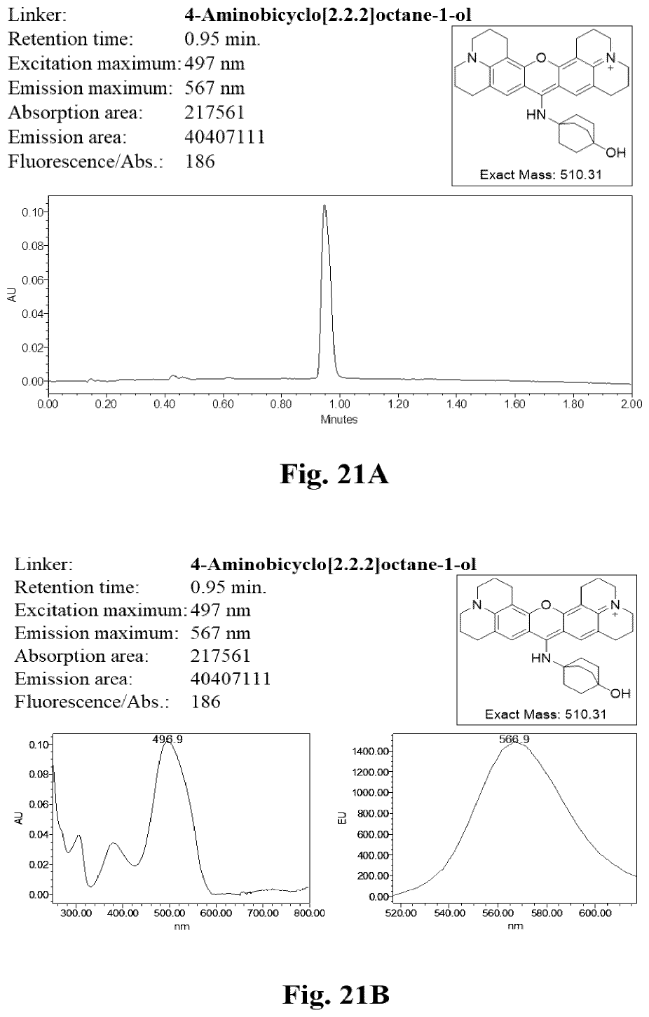

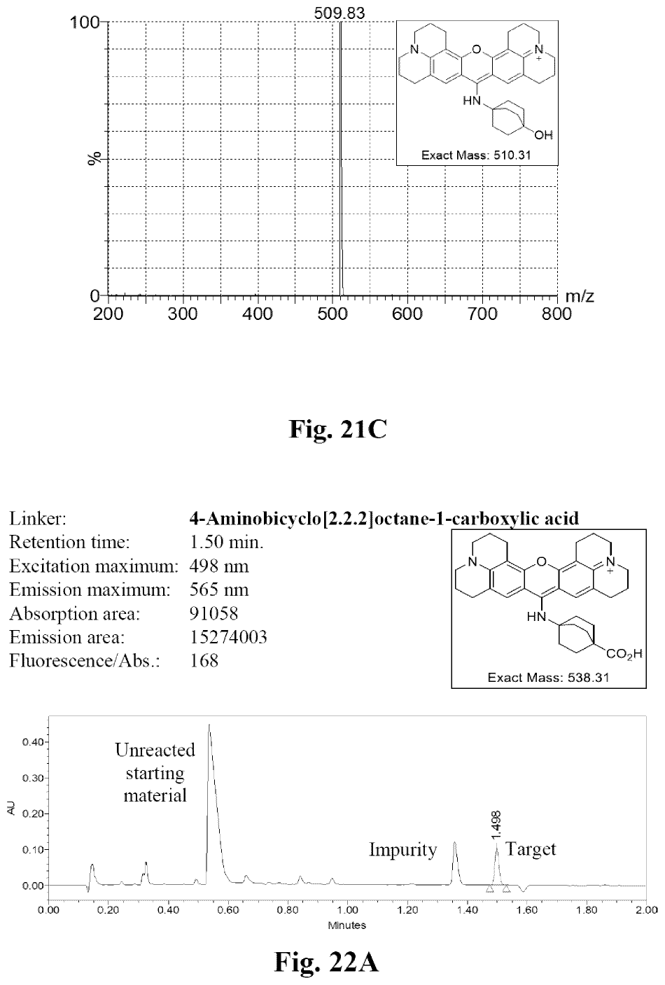

Results: Successful synthesis of compound 1q was demonstrated by the analytical data in FIG. 21 (A: Chromatograms; B: Excitation and emission spectra; C: Mass spectrum).

### Example 5: Thermostability of Fluorescence

A small sample of LSS dye in DMSO was diluted with TEAA buffer (0.1 M, pH 7.0, 0.5 mL) to a DMSO concentration of 10%. The fluorescence signal was recorded as a function of temperature by exciting the respective LSS dye at the excitation maximum and recording the fluorescence at the emission maximum from 25° C. to 100° C. at a heating rate of 1° C./min.

Results: The fluorescence as a function of temperature for compounds R, 1i, 1k, 1j, and 1n is shown in FIG. 24. The results show no significant decrease of fluorescence up to 100° C., which demonstrates the thermostability of fluorescence for the LSS dyes of the present disclosure. The somewhat more pronounced drift to higher fluorescence for compounds R and in could be explained by their increased solubility at higher temperature, since they are more hydrophobic compared to compounds 1i, 1k, 1j, which are carboxylic acids.

### Example 6: Direct Labeling of DNA with R800 Dye

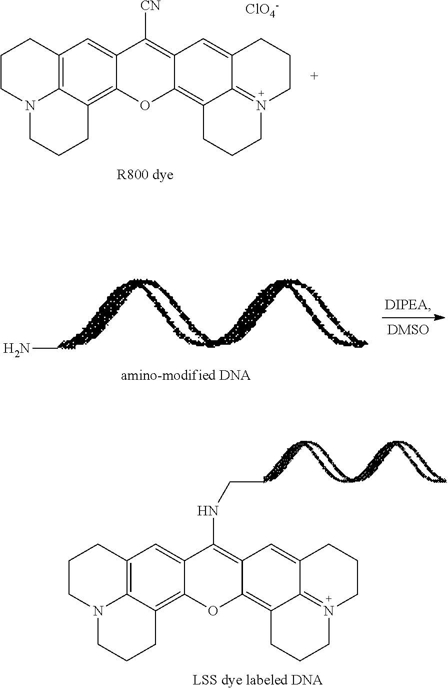

To enable solubility of the oligonucleotide in polar organic solvents, the amino-modified DNA was converted to the TEA salt via standard salt-exchange methods. The water was removed by lyophilization, and the residue redissolved in dry DMSO. After addition of DIPEA (1.0 eq., 100 nmol) and R800 perchlorate dye (5.0 eq., 500 nmol) the DNA concentration was 1.0 mM. The labeling reaction was allowed to progress for 67 h at RT. An analytical quantity of the reaction solution was analyzed by UPLC-MS to determine the fraction of labeled DNA (TEAA buffer, pH 7.0, 10-40% MeCN in 2.0 min.). Excess dye was removed by EtOH precipitation, and the labeled DNA was purified by preparative HPLC, using a biphasic gradient (10-25% MeCN, 5 min., and then 25-45% MeCN, 20 min.). After confirming the identity of the product by mass spectrometry, the labeled DNA was isolated by standard procedures at >95% purity.

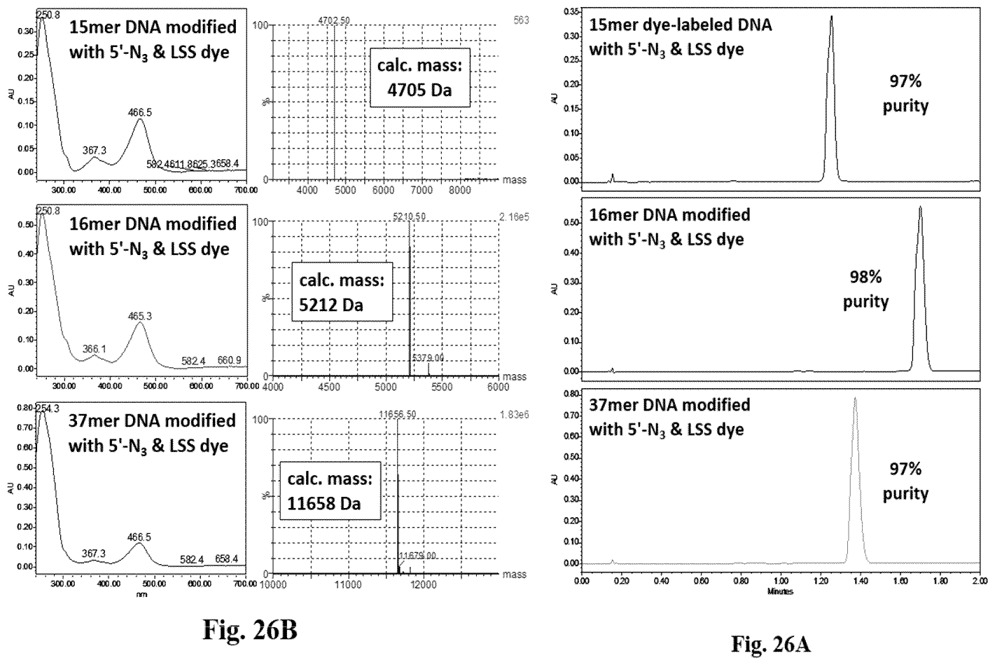

Results: The effectiveness of the direct-labeling method has been demonstrated by successful derivatization of a variety of amino-modified DNA probe sequences with R800. Analytical data for the labeling reaction of a TaqMan® probe sequence carrying an internal BHQ-2 is shown in FIG. 25. This labeled DNA has been used in Example 10. Analytical data for three 5′-azido-modified DNA sequences are shown in FIG. 26A (chromatograms) and FIG. 26B (absorption and mass spectra). These results illustrated that site-specific labeling of DNA could be achieved without the need for active ester or click-chemistry functionalities. With this approach the molecular configuration of the amino-liker of the DNA was incorporated to become an integral part of the dye structure.

### Example 7: Labeling of DNA with LSS Dye Carboxylic Acids

Following automated solid-phase synthesis and standard workup procedures, an analytical quantity of the unpurified DNA was analyzed with UPLC-MS to determine the fraction of amino-modified target sequence. The total DNA amount was determined with a spectrophotometer, using the calculated extinction coefficient of the DNA sequence at 260 nm.

DNA precipitation: The unpurified DNA as obtained from solid-phase synthesis was desalted against water with standard methods (NAP-25 or SPE) and dried with a rotary vacuum concentrator. In a reaction tube the DNA was redissolved in water (0.1 mL) with the aid of a warm water bath. Sodium iodide was added to a final concentration of 10.0 M. Absolute ethanol (200 proof, 30.0 mL) was added and the tube was vortexed. The suspension was centrifuged in a tabletop centrifuge at maximum speed (5.0 min.). The supernatant was carefully decanted and discarded. Excess sodium iodide was removed by washing the pellet with dry ethanol. The pellet was dried at high vacuum, redissolved in TEAB buffer (pH 8.5) for immediate use in labeling reactions.

Fluorescent dye carboxylic acid activation: In a reaction vial the carboxylic acid dye (1.0 eq.) was dissolved in dry DMSO (8 mM). DIPEA (2.0 eq.) was added, and the solution was briefly mixed. In a separate glass vial the tetrafluoroborate salt of DMT-MM (2.0 eq.) was weighed in, the dye carboxylic acid solution was added, followed by vigorous mixing until all solids were dissolved.

The reaction was shaken for 15 min. at RT.

Labeling reaction: The activated dye solution (3.0 eq.) was quickly mixed with the amino-modified DNA (1.0 eq. primary amine) and the labeling reaction was carried out in a reaction mixer for 30 min. at RT. The progress of the reaction was monitored by UPLC analysis, for which a sample of the reaction mixture (1.0 μL) was diluted with water (19.0 μL) prior to UPLC injection (7.0 μL).

Purification: The labeled DNA was purified with reversed-phase liquid chromatography using an appropriate gradient of TEAA buffer (0.1 M, pH 7.0) and MeCN. The combined product fractions were concentrated on a centrifugal vacuum concentrator and desalted by size-exclusion chromatography. The purified DNA probes (0.1 mM) were lyophilized and redissolved in TE buffer (10.0 mM Tris·HCl, 1.0 mM EDTA) for qPCR.

Results: Successful labeling of amino-modified DNA with LSS dye 1j was demonstrated by the analytical data in FIG. 27 (A: Chromatograms; B: Excitation and emission spectra; C: Mass spectrum). Since compound 1j was a racemic mixture of enantiomers, the labeled DNA was a mixture of diastereomers and separated as double peak.

### Example 8: Preparation of Dye-Labeled DNA Probes with Click-Chemistry

Dye labeled DNA probes can also be prepared by strain-promoted azide-alkyne cycloaddition between DNA-bound DBCO or BCN and azido-modified dye. DNA probes containing a 5′-DBCO modification, internal BHQ-2, and 3′-C3 extension blocker were prepared by solid-phase DNA synthesis and purification with standard methods. DNA (1.0 eq., 100 μM, 50 mM TEAA buffer, pH 7.0) and a dye azide (1.1 eq., 100 μM, 50 mM TEAA buffer, pH 7.0) were mixed and kept in a shaker for 2 h at 40° C. Any dye excess was removed by ethanol precipitation. UPLC analysis showed quantitative labeling of the DNA. The protocol above has been used to prepare several DNA conjugates with common fluorescent dyes. It is expected that LSS dyes with azido-linker will yield DNA-LSS dye conjugates in the same manner.

### Example 9: Preparation of Branched DNA Probes with Click-Chemistry

Branched DNA probes for thermal multiplexing were prepared by strain-promoted azide-alkyne cycloaddition between an oligonucleotide with 5′-BHQ-2 and internal DBCO modification, and a 5′-azido-modified oligonucleotide with a dye at the penultimate position (e.g., labeled DNA from Example 6). Both DNA sequences were mixed at a stoichiometric ratio of 1:1.1 in TEAA buffer (50 mM, pH 7.0) and kept in a reaction mixer for 2 h at 40° C. UPLC analysis showed quantitative click-reaction with some left-over DNA excess that was removed by HPLC purification.

### Example 10: PCR Amplification with Dye Labeled DNA Probes

All qPCR components were prepared with nuclease-free water. Reaction mixtures with a total volume of 50 μL were prepared by combining three components termed master mixture (20 μL), buffer mixture (20 μL), and dNTP mixture (10 μL). The master mixture contained tricine buffer (pH 8.2), manganese acetate, potassium acetate, glycerol, DMSO, detergent, target DNA (5000 copies/reaction), polymerase aptamer, forward and reverse primer DNA, and polymerase enzyme, and a TaqMan® probe. The dNTP mixture contained dATP, dCTP, dGTP (2.0 mM each), and dUTP (4.0 mM). Each qPCR with 5 μmol target was prepared as duplicate in the wells of a 96-well plate. The TaqMan® probe was a DNA sequence with large Stoke's shift (LSS) dye and BHQ-2 quencher that has been prepared in Example 6 (Analytical data in FIG. 25). For comparison, another qPCR contained a TaqMan® probe with the same sequence, which was labeled with Cy5.5. The plate was sealed and subjected to amplification cycles with a LightCycler® 480 System (Fritz Hoffmann-La Roche, Basel, Switzerland). The growth curves were analyzed from fluorescence data collected in the appropriate combination of excitation and emission channels. Results: FIG. 28 shows PCR growth curves for the TaqMan® probe with LSS and Cy5.5 dye that were measured in their respective optical channel as described in FIG. 2. Specifically, the fluorescence for the LSS-labeled probe was detected in channel RLS 1 (435 nm excitation and 580 nm emission), whereas the fluorescence of the Cy5.5-labeled probe was detected in the Cy5.5 channel (580 nm excitation and 700 nm emission). Both growth curves were overlaid in FIG. 28 for comparison. The LSS dye signal showed fluorescence signal than the traditional Cy5.5 dye. This experiment demonstrates the general applicability and compatibility of LSS dyes as bright reporters in TaqMan® PCR, and it is expected that other LSS dye variants in this disclosure will generate a qPCR signal in the same manner.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A compound having Formula (I): 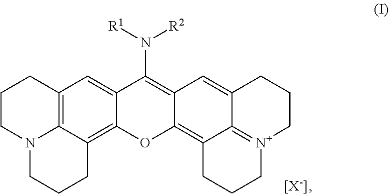
wherein
R¹ is H or a protecting group;
R² is a C₁-C₈ branched or unbranched alkyl, branched or unbranched heteroalkyl, or cycloalkyl group which is substituted with one or more of -Me, -Et, —CO₂⁻, —CO₂-(thiol reactive group), —CO₂-(amine reactive group), —CO₂-(carboxy reactive group), —C₂—CO₂⁻, —C₂—CO₂-(thiol reactive group), —C₂—CO₂-(amine reactive group), —C₂—CO₂-(carboxy reactive group), —OH, -phosphoramidite, —O-phosphoramidite, -D, a halogen, or a group capable of participating in a “click chemistry” reaction; and
[X]⁻ is a counter anion, provided when R² has a negative charge, [X]⁻ is not present.

2. The compound of claim 1, wherein the thiol reactive group is selected from the group consisting of a haloacetyl, a maleimide, an iodoacetamide, an aziridine, an acryloyl, an arylating agent, a vinylsulfone, a methanethiosulfonate, a pyridyl disulfide, and a TNB-thiol.

3. The compound of claim 1, wherein the amine reactive group is selected from the group consisting of an NHS ester, an isothiocyanate, an acyl azide, a sulfonyl chloride, a sulfodichlorophenol, pentafluorophenol, tetrafluorophenol, 4-sulfo-2,3,5,6-tetrafluorophenyl, an aldehyde, a glyoxal, an epoxide, an oxirane, a carbonate, an aryl halide, a fluorophenol ester, a sulfochlorophenol, a carbodiimide, a phthalimide, a benzotriazole, an imidoester, and an anhydride.

4. The compound of claim 1, wherein the carbonyl-reactive group is selected from the group consisting of a hydrazine, a hydrazine derivative, and an amine.

5. The compound of claim 1, wherein R² is selected from the group consisting of a C₁-C₈ branched or unbranched alkyl, branched or unbranched heteroalkyl, or cycloalkyl group which is substituted with a —CO₂-maleimide or a —C₂—CO₂— maleimide; a C₁-C₈ branched or unbranched alkyl, branched or unbranched heteroalkyl, or cycloalkyl group which is substituted with a —CO₂—NHS ester or a —C₂—CO₂—NHS ester; and a C₁-C₈ branched or unbranched alkyl, branched or unbranched heteroalkyl, or cycloalkyl group which is substituted with a —CO₂-hydrazine or a —C₂—CO₂— hydrazine.

6. The compound of claim 1, wherein the group capable of participating in the “click chemistry reaction” is selected from the group consisting of a bicyclo[6.1.0]nonyne) group (“BCN”), dibenzocyclooctyne (“DBCO”), alkene, trans-cycloctene (“TCO”), maleimide, an aldehyde, a ketone, an azide, a tetrazine, a thiol, a 1,3-nitrone, a hydrazine, and a hydroxylamine.

7. The compound of claim 6, wherein R² is a C₁-C₆ branched or unbranched alkyl group substituted with BCN, DBCO or TCO.

8. The compound of claim 1, wherein R² is a C₁-C₆ branched or unbranched alkyl group substituted with one or more of -Me, -Et, —CO₂⁻, —OH, -D, or a halogen; or
wherein R² is selected from the group consisting of:

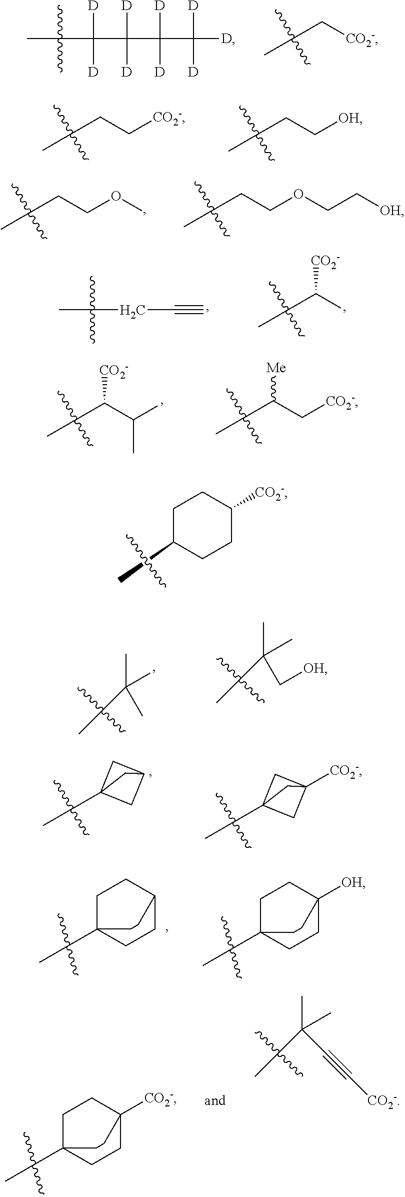

9. The compound of claim 1, wherein R¹ is a protecting group; and wherein R² comprises-phosphoramidite or —O-phosphoramidite.

10-12. (canceled)

13. A compound having Formula (IA): 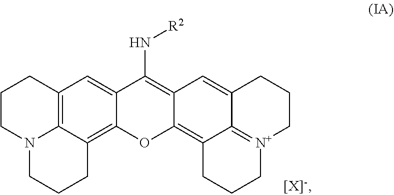
wherein R² is a C₁-C₈ branched or unbranched alkyl, branched or unbranched heteroalkyl, or cycloalkyl group which is substituted with one or more of -Me, -Et, —CO₂⁻, —CO₂-(thiol reactive group), —CO₂-(amine reactive group), —CO₂-(carboxy reactive group), —C₂—CO₂⁻, —C₂—CO₂-(thiol reactive group), C₂—CO₂-(amine reactive group), —C₂—CO₂-(carboxy reactive group), —OH, phosphoramidite, —O-phosphoramidite, -D, a halogen, or a group capable of participating in a “click chemistry” reaction; and [X]⁻ is a counter anion, provided when R₂ has a negative charge, [X]⁻ is not present.

14. The compound of claim 13, wherein R² is selected from the group consisting of a C₁-C₈ branched or unbranched alkyl, branched or unbranched heteroalkyl, or cycloalkyl group which is substituted with a —CO₂-maleimide or a —C₂—CO₂-maleimide; a C₁-C₈ branched or unbranched alkyl, branched or unbranched heteroalkyl, or cycloalkyl group which is substituted with a —CO₂—NHS ester or a —C₂—CO₂—NHS ester; and a C₁-C₈ branched or unbranched alkyl, branched or unbranched heteroalkyl, or cycloalkyl group which is substituted with a —CO₂-hydrazine or a —C₂—CO₂-hydrazine; or
wherein R² is a C₁-C₈ branched or unbranched alkyl group substituted with a group capable of participating in a “click chemistry” reaction; or
wherein R² is a C₁-C₈ branched or unbranched alkyl group substituted with one or more of -Me, -Et, —CO₂⁻, —OH, a halogen, or -D; or
wherein R² is -phosphoramidite or —O-phosphoramidite; or
wherein R² is selected from the group consisting of:

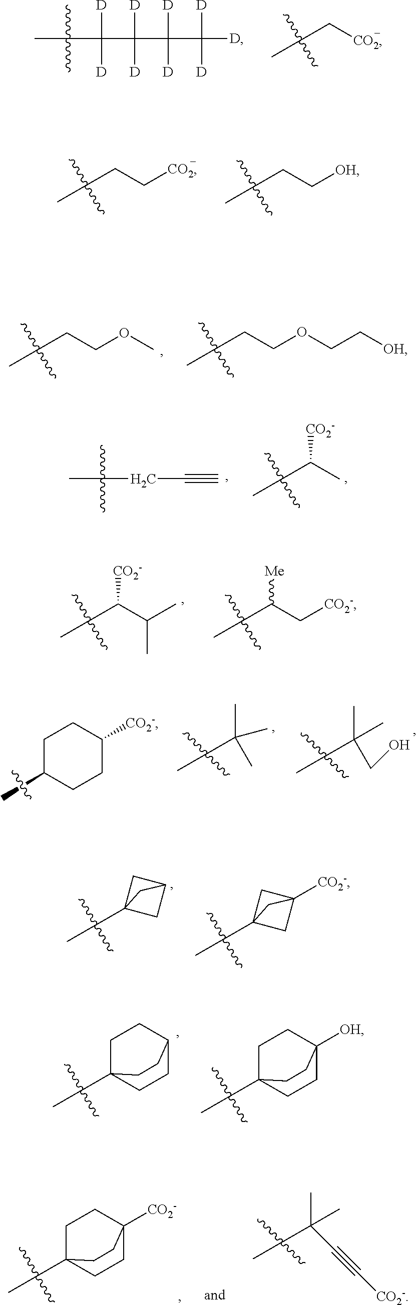

15. The compound of claim 13, wherein the compound has a Stokes shift of at least about 70 nm.

16. The compound of claim 13, wherein the compound is thermally stable over a temperature ranging from about 25° C. to about 100° C.

17. A conjugate comprising (i) a specific binding entity, and (ii) a dye moiety derived from any one of the compounds of claim 1.

18. A conjugate having Formula (II): 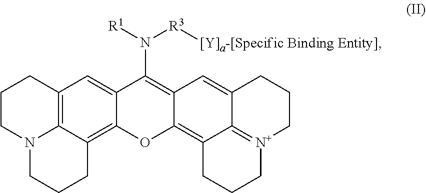
wherein
R¹ is H or a protecting group;
R³ is a C₁-C₈ alkyl, heteroalkyl, or cycloalkyl group which is substituted with one or more of -Me, -Et, —CO₂—, —C₂—CO₂—, -D, or a halogen;
the “Specific Binding Entity” is an oligonucleotide or a protein;
Y is a branched or unbranched, substituted or unsubstituted, saturated or unsaturated aliphatic or aromatic group having between 2 and about 40 carbon atoms, and optionally having one or more heteroatoms selected from O, N, or S; and
a is 0, 1, or 2.

19. The conjugate of claim 18, wherein Y is a branched or unbranched, linear, or cyclic, substituted or unsubstituted, saturated or unsaturated, group having between 2 and about 25 carbon atoms, and optionally having one or more heteroatoms selected from O, N, or S.

20. The conjugate of claim 18, wherein Y has the structure of Formula (IIIC): 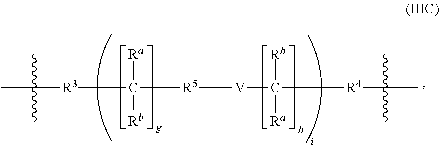
wherein each of R³ and R⁴ are independently a bond or a group selected from carbonyl, amide, imide, ester, ether, —NH, —N—, thione, or thiol;
R⁵ is a bond, a C₁-C₁₂ alkyl or heteroalkyl group including, and wherein R⁵ may include a carbonyl, an imine, or a thione;
R^{a} and R^{b} are independently H or methyl;
g and h are independently 0 or an integer ranging from 1 to 4;
i is 0, 1 or 2.

21-28. (canceled)

29. A conjugate having Formula (V): 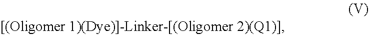
wherein
Oligomers 1 and 2 are each different and are oligonucleotides having between about 5 mer and about 30 mer;
Dye is derived from the compound of claim 1;
Q1 is a quencher; and
Linker is a substituted or unsubstituted aliphatic, heteroaliphatic, aromatic, or heteroaromatic group having between about 5 and about 30 carbon atoms.

30-37. (canceled)

38. A method for amplification and detection of a target nucleic acid in a sample comprising the steps of:
(a) contacting the sample containing the target nucleic acid in a single reaction vessel with
(i) one pair of oligonucleotide primers, each oligonucleotide primer capable of hybridizing to opposite strands of a subsequence of the target nucleic acid;
(ii) an oligonucleotide probe that comprises an annealing portion and a tag portion, wherein the tag portion comprises a nucleotide sequence non-complementary to the target nucleic acid sequence, wherein the annealing portion comprises a nucleotide sequence at least partially complementary to the target nucleic acid sequence and hybridizes to a region of the subsequence of the target nucleic acid that is bounded by the pair of oligonucleotide primers, wherein the probe further comprises an interactive dual label comprising a dye derived from the compound of claim 13, located on the tag portion and a first quencher moiety located on the annealing portion and wherein the dye is separated from the first quencher moiety by a nuclease susceptible cleavage site; and
wherein prior to step (b), the tag portion is reversibly bound in a temperature-dependent manner to a quenching oligonucleotide comprising a nucleotide sequence at least partially complementary to the tag portion of the oligonucleotide probe and binds to the tag portion by hybridization, wherein the quenching oligonucleotide comprises at least a second quencher moiety capable of quenching the dye on the tag portion when the quenching oligonucleotide is bound to the tag portion;
(b) following step (a), amplifying the target nucleic acid by polymerase chain reaction (PCR) using a nucleic acid polymerase having 5′ to 3′ nuclease activity such that during an extension step of each PCR cycle, the nuclease activity of the polymerase allows cleavage and separation of the tag portion from the first quencher moiety on the annealing portion of the probe;
(c) measuring one or more signals from the dye at a first temperature at which the quenching oligonucleotide is bound to the tag portion;
(d) measuring one or more signals from the dye at a second temperature, which is higher than the first temperature, at which the quenching oligonucleotide is not bound to the tag portion;
(e) obtaining a calculated signal value by subtracting a median or average of the one or more signals detected at the first temperature from a median or average of the one or more signals detected at the second temperature;
whereby a calculated signal value that is higher than a threshold signal value allows determination of the presence of the target nucleic acid.

39. The method of claim 38, wherein the one or more nucleotide modifications is selected from the group consisting of Locked Nucleic Acid (LNA), Peptide Nucleic Acid (PNA), Bridged Nucleic Acid (BNA), 2′-O alkyl substitution, L-enantiomeric nucleotide, and combinations thereof.

40-41. (canceled)
